(19) United States
(12) Patent Application Publication
(10) Pub. No.: US 2024/0002351 A1
(43) Pub. Date: Jan. 4, 2024

(54) QUINAZOLIN-4-ONE AND THIENO[2,3-D]PYRIMIDIN-4-ONE INHIBITORS OF ERBB4 (HER4) FOR USE IN THE TREATMENT OF CANCER

(71) Applicant: UNIVERSITEIT ANTWERPEN, Antwerpen (BE)

(72) Inventors: Vincent Frans Maria SEGERS, Berchem (BE); Gilles Willy DE KEULENAER, Boechout (BE); Eline FEYEN, Borsbeek (BE); Hans Louis Jos DE WINTER, Schilde (BE)

(73) Assignee: UNIVERSITEIT ANTWERPEN, Antwerpen (BE)

(21) Appl. No.: 18/276,532

(22) PCT Filed: Mar. 4, 2022
(86) PCT No.: PCT/EP2022/055561

§ 371 Date: Aug. 9, 2023

(30) Foreign Application Priority Data
Mar. 4, 2021 (EP) 21160742.9
Nov. 4, 2021 (EP) 21206420.8

(51) Int. Cl. C07D 239/95; C07D 495/04; C07D 403/12

(52) U.S. Cl. CPC C07D 239/95; C07D 403/12; C07D 495/04

(57) ABSTRACT

The present invention relates to a compound of formula (I) or a stereoisomer, or tautomer, for use in the prevention or treatment of a disease associated with the activation of receptor tyrosine-protein kinase (erbB4). In formula (I), wherein n, A¹, A², A³, A⁴, A⁵, R¹, R², R³, and R⁴ have the same meaning as that defined in the claims and the description. The present invention also relates uses of such compounds for the prevention and/or treatment of a disease associated with the activation of receptor tyrosine-protein kinase (erbB-4) such as heart failure, metabolic disorders, inflammatory disorders, fibrotic disorders, and cancer. The present invention also provides pharmaceutical compositions comprising such compounds, as well as the use of the compounds as a medicament.

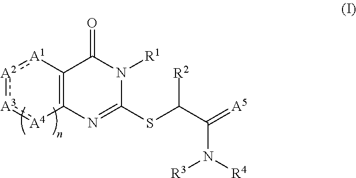

### FIELD OF THE INVENTION

The present invention relates to compounds for use as modulators of ERBB4 as an alternative for neuregulin in the treatment of diseases associated with the activation of receptor tyrosine protein kinase (ERBB4), such as heart failure, metabolic disorders, inflammatory disorders, fibrotic disorders, and cancer. The present invention also provides pharmaceutical compositions comprising such compounds, as well as the use of the compounds as a medicament.

### BACKGROUND OF THE INVENTION

Neuregulin-1 (NRG-1) belongs to the epidermal growth factor superfamily and is important in cardiac development, biology, and disease. Intracellular signaling is initiated when NRG-1 binds to one of two tyrosine kinase receptors (ERBB3 or ERBB4), which induces formation of dimers (with each other or with ERBB2) that couple to intracellular signaling cascades.

Recombinant NRG-1 (rNRG-1) has been evaluated as a potential therapy in many animal models of cardiac disease, including myocardial infarction, ischemia/reperfusion injury, diabetic cardiomyopathy, myocarditis, and chronic rapid pacing. Intravenous (IV) administration of rNRG-1 has positive effects on cardiac function in rats after coronary artery ligation, and on cardiac function in rats with diabetic cardiomyopathy. Reduced fibrosis in a swine model of cardiomyopathy has also been demonstrated after treatment with NRG-1. The cardioprotective effects of NRG-1 are almost exclusively based on its functioning as an agonist on ERBB4. Effects of NRG-1 on cardiomyocytes increase in transgenic mice overexpressing ERBB4 and decrease in mice with ERBB4 deletion. ERBB4 is a unique member of the ERBB family of receptor tyrosine kinases because it is the only one with growth inhibiting properties on tumor cells. It has also been shown that long-term administration of NRG-1 does not induce neoplastic growth.

NRG-1-based therapies are under development. Two Phase II clinical trials showed that daily infusions of rhNRG-1 were safe and well tolerated in patients with stable chronic heart failure (CHF). In a randomized Phase II double-blind study in patients with CHF, participants were given daily infusions of rhNRG-1 or a placebo for 10 days; at day 30, rhNRG-1 significantly increased left ventricular ejection fraction (LVEF). Another clinical trial demonstrated improved hemodynamics in patients with CHF who received daily infusions of rhNRG-1. Currently, a large Phase III trial is recruiting patients to evaluate the efficacy of rhNRG-1 in 1600 CHF patients (NCT03388593).

NRG-1 is the natural ligand of ERBB4 but its use as a drug has serious disadvantages. Because rNRG-1 is a protein, it can only be administered by parenteral administration (e.g. IV) in a hospital setting. In the current clinical trials, NRG-1 is administered over the course of 6-8 h by IV route in the hospital. This route of administration limits the frequency and total number of applications. This is even more problematic in chronic diseases such as CHF. The fact that rNRG-1 has to be delivered IV not only limits applicability, but might also limit efficacy because patients are only treated for a limited number of days.

Thus there is a need in the art for small molecules that exert the same physiological effects as NRG-1, but could circumvent the problems encountered with the administration of rNRG-1 and could represent a novel therapy for heart failure, fibrotic disorders, metabolic disorders, and inflammatory disorders.

### SUMMARY OF THE INVENTION

The present invention is based on the unexpected finding that at least one of the above-mentioned objectives can be attained by small molecules.

The present invention provides compounds which have been shown to modulate receptor tyrosine-protein kinase (ERBB4). The modulator compounds are capable of binding and activating to ERBB4. Advantageously, the modulator compounds exhibit allosteric modulating capacities when given together with the endogenous ligand Neuregulin-1 (NRG-1). The modulator compounds are capable of inhibiting or additively enhancing the activating effect of NRG-1 on ERBB4. ERBB4 modulation has been shown to be beneficial in chronic diseases such as heart failure, chronic diabetic nephrophathy, and dermal-, pulmonary- and myocardial fibrosis. Accordingly, the compounds of the invention are suitable for use as medicaments, more particularly in the treatment and/or prevention of diseases such as chronic diseases such as heart failure, chronic diabetic nephrophathy, and dermal-, pulmonary- and myocardial fibrosis. A first aspect of the present invention provides a compound of formula (I) or a stereoisomer, or tautomer thereof,

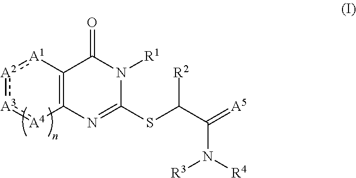

wherein,
each dotted line represents an optional double bond whereby maximally 2 non-adjacent dotted lines can form a double bond;
n is an integer selected from 0, 1, or 2;
A¹ is selected from the group consisting of CR⁵, S, N, NR⁶, and O;
A² is selected from the group consisting of CR⁵, S, N, NR⁶, and O;
A³ is selected from the group consisting of CR⁵, S, N, NR⁶, and O;
A⁴ is selected from the group consisting of CR⁵, S, N, NR⁶, and O;
wherein at most two of A¹ to A⁴ is selected from S, N, NR⁶, and O;
A⁵ is 0 or NR⁷;
R¹ is selected from the group consisting of alkyl, cycloalkyl, alkenyl, aryl, arylalkyl, heterocyclyl, and heteroaryl; wherein said alkyl, cycloalkyl, alkenyl, aryl, arylalkyl, heterocyclyl, or heteroaryl can be unsubstituted or substituted with one or more Z¹;
R² is selected from the group consisting of hydrogen, alkyl, cycloalkyl, aryl, arylalkyl, heterocyclyl, and heteroaryl; wherein said alkyl, cycloalkyl, aryl, arylalkyl, heterocyclyl, or heteroaryl can be unsubstituted or substituted with one or more Z²;
R³ is selected from the group consisting of hydrogen, cycloalkyl, aryl, alkyl, haloalkyl, haloalkyloxy, arylalkyl, heterocyclyl, heteroaryl, —C(O)R⁸, —CO₂R⁹, —C(O)NR⁹R¹⁰, —S(O)₂R⁹, and —SO₂NR⁹R¹⁰;
wherein said cycloalkyl, aryl, alkyl, arylalkyl, heterocyclyl, or heteroaryl can be unsubstituted or substituted with one or more Z³;
or R² and R³ together with the atom to which they are attached form a saturated or unsaturated 5-, 6-, 7-, 8- or 9-membered ring;
R⁴ is selected from the group consisting of hydrogen, cycloalkyl, aryl, alkyl, haloalkyl, haloalkyloxy, arylalkyl, heterocyclyl, and heteroaryl; wherein said cycloalkyl, aryl, alkyl, haloalkyl, haloalkyloxy, arylalkyl, heterocyclyl, or heteroaryl can be unsubstituted or substituted with one or more Z⁴;
or R³ and R⁴ together with the nitrogen atom to which they are attached form a saturated or unsaturated 5-, 6-, 7-, 8-, 9-, 10- or 11-membered nitrogen-containing monocycle or bicycle;
R⁵ is selected from the group consisting of hydrogen, alkyl, cycloalkyl, halo, aryl, arylalkyl, heterocyclyl, heteroaryl, hydroxyl, —OR^{B}, cyano, amino, nitro, haloalkyl, haloalkyloxy, —C(O)R⁸, —NR⁸R⁹, —CO₂R⁹, —C(O)NR⁹R¹⁰, —S(O)₂R⁹, —S(O)₂NR⁹R¹⁰, —NR⁹C(O)R¹⁰ and —NR⁹S(O)₂R¹⁰;
or wherein two R⁵ together with the atom to which they are attached can form a saturated or unsaturated 5-, 6-, 7-, 8-, 9- or 10-membered ring;
wherein said ring, alkyl, cycloalkyl, aryl, arylalkyl, heterocyclyl, or heteroaryl can be unsubstituted or substituted with one or more Z⁵;
R⁶ is selected from the group consisting of hydrogen, alkyl, cycloalkyl, aryl, arylalkyl, heterocyclyl, heteroaryl, and —S(O)₂R⁹; wherein said alkyl, cycloalkyl, aryl, arylalkyl, heterocyclyl, or heteroaryl can be unsubstituted or substituted with one or more Z⁶;
R⁷ is selected from the group consisting of alkyl, cycloalkyl, aryl, arylalkyl, heterocyclyl, and heteroaryl; wherein said alkyl, cycloalkyl, aryl, arylalkyl, heterocyclyl, or heteroaryl can be unsubstituted or substituted with one or more Z⁷;
or R³ and R⁷ together with the atom to which they are attached form an unsaturated 5-, 6-, 7-, 8-, 9-, 10- or 11-membered nitrogen-containing monocycle or bicycle;
each R⁸ is independently selected from the group consisting of, alkyl, aryl, cycloalkyl, arylalkyl, heterocyclyl, heteroaryl;
each R⁹ is independently selected from the group consisting of hydrogen, alkyl, aryl, cycloalkyl, arylalkyl, heterocyclyl, heteroaryl;
each R¹⁰ is independently selected from the group consisting of hydrogen, alkyl, aryl, cycloalkyl, arylalkyl, heterocyclyl, heteroaryl;
each Z¹ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z² is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z³ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z⁴ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z⁵ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z⁶ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z⁷ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
or a solvate, hydrate, pharmaceutically acceptable salt, or prodrug thereof. More particularly, the invention provides these compounds for use in the prevention or treatment of a disease associated with the activation of receptor tyrosine-protein kinase (ERBB4).

According to particular embodiments, the present invention provides compounds of formula (I), and any subgroup thereof such as (IAA), (IA), (IB), (IC), (IIA), (IIB), (IIC), (IID), wherein,
A¹ is CR⁵, A² is CR⁵, A³ is CR⁵ or S, A⁴ is CR⁵ and A⁵ is O;
R¹ is selected from the group consisting of C₁₋₆alkyl, C₃₋₁₂cycloalkyl, C₂₋₆alkenyl, C₆₋₁₂aryl, C₆₋₁₂arylC₁₋₆alkyl and heteroaryl; wherein said groups can be unsubstituted or substituted with one, two or three Z¹;
R² is selected from the group consisting of hydrogen, C₁₋₆alkyl, C₃₋₁₂cycloalkyl and C₆₋₁₂aryl;
R³ is selected from the group consisting of hydrogen, C₃₋₁₂cycloalkyl, C₆₋₁₂aryl, C₁₋₆alkyl, haloC₁₋₄ alkyl, haloC₁₋₄alkyloxy, C₆₋₁₂arylC₁₋₆alkyl, heterocyclyl, heteroaryl, C₁₋₄alkylcarbonyl, C₃₋₁₂cycloalkylcarbonyl, C₆₋₁₂arylcarbonyl, C₁₋₄alkyloxycarbonyl, C₃₋₁₂cycloalkyloxycarbonyl, C₆₋₁₂aryloxycarbonyl, aminocarbonyl, mono-C₁₋₄alkylaminocarbonyl, and mono-C₃₋₁₂cycloalkylaminocarbonyl; wherein said groups can be unsubstituted or substituted with one, two or three Z³;
or R² and R³ together with the atom to which they are attached form a saturated unsaturated 6-, 7-, or 8-membered ring;
R⁴ is selected from the group consisting of hydrogen, C₃₋₁₀cycloalkyl, C₆₋₁₂aryl, and haloC₁₋₄alkyl;
or R³ and R⁴ together with the nitrogen atom to which they are attached form a saturated 7-, 8-, 9- or 10-membered nitrogen-containing monocycle or bicycle;
R⁵ is selected from the group consisting of hydrogen, C₁₋₆alkyl, C₃₋₁₂cycloalkyl, halo, C₆₋₁₂aryl, C₆₋₁₂arylC₁₋₆alkyl, heterocyclyl, heteroaryl, hydroxyl, C₁₋₆alkyloxy, C₃₋₂cycloalkyloxy, C₆₋₁₂aryloxy, heterocyclyloxy, heteroaryloxy, cyano, amino, nitro, haloC₁₋₄alkyl, haloC₁₋₄alkyloxy, C₁₋₄ alkylcarbonyl, C₃₋₁₂cycloalkylcarbonyl, C₆₋₁₂arylcarbonyl, mono-C₁₋₄alkylamino, mono-C₃₋₁₂cycloalkylamino, mono-C₆₋₁₂arylamino, hydroxycarbonyl, C₁₋₄alkyloxycarbonyl, C₃₋₁₂cycloalkyloxycarbonyl, C₆₋₁₂aryloxycarbonyl, aminocarbonyl, mono-C₁₋₄alkylaminocarbonyl, mono-C₃₋₁₂cycloalkylaminocarbonyl, S(O)₂H, C₁₋₄alkylsulfonyl, —SO₂N H₂, mono-C₁₋₄ alkylaminosulfonyl, C₁₋₄alkylcarbonylamino, and C₁₋₄alkylsulfonylamino;
or wherein two R⁵ together with the atom to which they are attached can form a saturated or unsaturated 5-, 6-, or 7-membered ring;
wherein said ring, C₁₋₆alkyl, C₃₋₁₂cycloalkyl, C₆₋₁₂aryl, C₆₋₁₂arylC₁₋₆alkyl, heterocyclyl, or heteroaryl can be unsubstituted or substituted with one, two or three Z⁵;
R⁶ is selected from the group consisting of hydrogen, C₁₋₆alkyl and C₃₋₁₂cycloalkyl;
R⁷ is selected from the group consisting of C₁₋₆alkyl, C₃₋₁₂cycloalkyl, C₆₋₁₂aryl, C₆₋₁₂arylalkyl, heterocyclyl, and heteroaryl;
or R³ and R⁷ together with the atom to which they are attached form an unsaturated 7-, 8-, 9-, or 10-membered nitrogen-containing monocycle or bicycle. In particular embodiments, these compounds are envisaged for use as a medicament. In further embodiments the compounds are envisaged for use in the prevention or treatment of a disease associated with the activation of receptor tyrosine-protein kinase (ERBB4) as described herein.

Another aspect of the present invention provides a compound of formula (I) or a stereoisomer, or tautomer thereof,

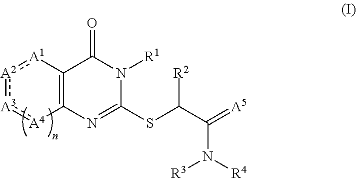

wherein,
each dotted line represents an optional double bond whereby maximally 2 non-adjacent dotted lines can form a double bond;
n is an integer selected from 0, 1, or 2;
A¹ is selected from the group consisting of CR⁵, S, N, NR⁶, and O;
A² is selected from the group consisting of CR⁵, S, N, NR⁶, and O;
A³ is selected from the group consisting of CR⁵, S, N, NR⁶, and O;
A⁴ is selected from the group consisting of CR⁵, S, N, NR⁶, and O;
wherein at most two of A¹ to A⁴ is selected from S, N, NR⁶, and O;
A⁵ is 0 or NR⁷;
R¹ is selected from the group consisting of alkyl, cycloalkyl, alkenyl, aryl, arylalkyl, heterocyclyl, and heteroaryl; wherein said alkyl, cycloalkyl, alkenyl, aryl, arylalkyl, heterocyclyl, or heteroaryl can be unsubstituted or substituted with one or more Z¹;
R² is selected from the group consisting of hydrogen, alkyl, cycloalkyl, aryl, arylalkyl, heterocyclyl, and heteroaryl; wherein said alkyl, cycloalkyl, aryl, arylalkyl, heterocyclyl, or heteroaryl can be unsubstituted or substituted with one or more Z²;
R³ is selected from the group consisting of hydrogen, cycloalkyl, aryl, alkyl, haloalkyl, haloalkyloxy, arylalkyl, heterocyclyl, heteroaryl, —C(O)R⁸, —CO₂R⁹, —C(O)NR⁹R¹⁰, —S(O)₂R⁹, and —SO₂NR⁹R¹⁰;
wherein said cycloalkyl, aryl, alkyl, arylalkyl, heterocyclyl, or heteroaryl can be unsubstituted or substituted with one or more Z³;
or R² and R³ together with the atom to which they are attached form a saturated or unsaturated 5-, 6-, 7-, 8- or 9-membered ring;
R⁴ is selected from the group consisting of hydrogen, cycloalkyl, aryl, alkyl, haloalkyl, haloalkyloxy, arylalkyl, heterocyclyl, and heteroaryl; wherein said cycloalkyl, aryl, alkyl, haloalkyl, haloalkyloxy, arylalkyl, heterocyclyl, or heteroaryl can be unsubstituted or substituted with one or more Z⁴;
or R³ and R⁴ together with the nitrogen atom to which they are attached form a saturated or unsaturated 5-, 6-, 7-, 8-, 9-, 10- or 11-membered nitrogen-containing monocycle or bicycle;
R⁵ is selected from the group consisting of hydrogen, alkyl, cycloalkyl, halo, aryl, arylalkyl, heterocyclyl, heteroaryl, hydroxyl, —OR⁸, cyano, amino, nitro, haloalkyl, haloalkyloxy, —C(O)R⁸, —NR⁸R⁹, —CO₂R⁹, —C(O)NR⁹R¹⁰, —S(O)₂R⁹, —S(O)₂NR⁹R¹⁰, —NR⁹C(O)R¹⁰, and —NR⁹S(O)₂R¹⁰;
or wherein two R⁵ together with the atom to which they are attached can form a saturated or unsaturated 5-, 6-, 7-, 8-, 9- or 10-membered ring;
wherein said ring, alkyl, cycloalkyl, aryl, arylalkyl, heterocyclyl, or heteroaryl can be unsubstituted or substituted with one or more Z⁵;
R⁶ is selected from the group consisting of hydrogen, alkyl, cycloalkyl, aryl, arylalkyl, heterocyclyl, heteroaryl, and —S(O)₂R⁹; wherein said alkyl, cycloalkyl, aryl, arylalkyl, heterocyclyl, or heteroaryl can be unsubstituted or substituted with one or more Z⁶;
R⁷ is selected from the group consisting of alkyl, cycloalkyl, aryl, arylalkyl, heterocyclyl, and heteroaryl; wherein said alkyl, cycloalkyl, aryl, arylalkyl, heterocyclyl, or heteroaryl can be unsubstituted or substituted with one or more Z⁷;
or R³ and R⁷ together with the atom to which they are attached form an unsaturated 5-, 6-, 7-, 8-, 9-, 10- or 11-membered nitrogen-containing monocycle or bicycle;
each R⁸ is independently selected from the group consisting of, alkyl, aryl, cycloalkyl, arylalkyl, heterocyclyl, heteroaryl;
each R⁹ is independently selected from the group consisting of hydrogen, alkyl, aryl, cycloalkyl, arylalkyl, heterocyclyl, heteroaryl;
each R¹⁰ is independently selected from the group consisting of hydrogen, alkyl, aryl, cycloalkyl, arylalkyl, heterocyclyl, heteroaryl;
each Z¹ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z² is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z³ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z⁴ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z⁵ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z⁶ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z⁷ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro; or a solvate, hydrate, pharmaceutically acceptable salt, or prodrug thereof for use as a medicament.

Yet another aspect of the present invention provides a pharmaceutical composition comprising a compound of formula (I) or a stereoisomer, or tautomer thereof,

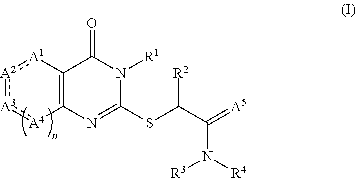

wherein,
each dotted line represents an optional double bond whereby maximally 2 non-adjacent dotted lines can form a double bond;
n is an integer selected from 0, 1, or 2;
A¹ is selected from the group consisting of CR⁵, S, N, NR⁶, and O;
A² is selected from the group consisting of CR⁵, S, N, NR⁶, and O;
A³ is selected from the group consisting of CR⁵, S, N, NR⁶, and O;
A⁴ is selected from the group consisting of CR⁵, S, N, NR⁶, and O;
wherein at most two of A¹ to A⁴ is selected from S, N, NR⁶, and O;
A⁵ is 0 or NR⁷;
R¹ is selected from the group consisting of alkyl, cycloalkyl, alkenyl, aryl, arylalkyl, heterocyclyl, and heteroaryl; wherein said alkyl, cycloalkyl, alkenyl, aryl, arylalkyl, heterocyclyl, or heteroaryl can be unsubstituted or substituted with one or more Z¹;
R² is selected from the group consisting of hydrogen, alkyl, cycloalkyl, aryl, arylalkyl, heterocyclyl, and heteroaryl; wherein said alkyl, cycloalkyl, aryl, arylalkyl, heterocyclyl, or heteroaryl can be unsubstituted or substituted with one or more Z²;
R³ is selected from the group consisting of hydrogen, cycloalkyl, aryl, alkyl, haloalkyl, haloalkyloxy, arylalkyl, heterocyclyl, heteroaryl, —C(O)R⁸, —CO₂R⁹, —C(O)NR⁹R¹⁰, —S(O)₂R⁹, and —SO₂NR⁹R¹⁰;
wherein said cycloalkyl, aryl, alkyl, arylalkyl, heterocyclyl, or heteroaryl can be unsubstituted or substituted with one or more Z³;
or R² and R³ together with the atom to which they are attached form a saturated or unsaturated 5-, 6-, 7-, 8- or 9-membered ring;
R⁴ is selected from the group consisting of hydrogen, cycloalkyl, aryl, alkyl, haloalkyl, haloalkyloxy, arylalkyl, heterocyclyl, and heteroaryl; wherein said cycloalkyl, aryl, alkyl, haloalkyl, haloalkyloxy, arylalkyl, heterocyclyl, or heteroaryl can be unsubstituted or substituted with one or more Z⁴;
or R³ and R⁴ together with the nitrogen atom to which they are attached form a saturated or unsaturated 5-, 6-, 7-, 8-, 9-, 10- or 11-membered nitrogen-containing monocycle or bicycle;
R⁵ is selected from the group consisting of hydrogen, alkyl, cycloalkyl, halo, aryl, arylalkyl, heterocyclyl, heteroaryl, hydroxyl, —OR^{B}, cyano, amino, nitro, haloalkyl, haloalkyloxy, —C(O)R⁸, —NR⁸R⁹, —CO₂R⁹, —C(O)NR⁹R¹⁰, —S(O)₂R⁹, —S(O)₂NR⁹R¹⁰, —NR⁹C(O)R¹⁰ and —NR⁹S(O)₂R¹⁰;
or wherein two R⁵ together with the atom to which they are attached can form a saturated or unsaturated 5-, 6-, 7-, 8-, 9- or 10-membered ring;
wherein said ring, alkyl, cycloalkyl, aryl, arylalkyl, heterocyclyl, or heteroaryl can be unsubstituted or substituted with one or more Z⁵;
R⁶ is selected from the group consisting of hydrogen, alkyl, cycloalkyl, aryl, arylalkyl, heterocyclyl, heteroaryl, and —S(O)₂R⁹; wherein said alkyl, cycloalkyl, aryl, arylalkyl, heterocyclyl, or heteroaryl can be unsubstituted or substituted with one or more Z⁶;
R⁷ is selected from the group consisting of alkyl, cycloalkyl, aryl, arylalkyl, heterocyclyl, and heteroaryl; wherein said alkyl, cycloalkyl, aryl, arylalkyl, heterocyclyl, or heteroaryl can be unsubstituted or substituted with one or more Z⁷;
or R³ and R⁷ together with the atom to which they are attached form an unsaturated 5-, 6-, 7-, 8-, 9-, 10- or 11-membered nitrogen-containing monocycle or bicycle;
each R⁸ is independently selected from the group consisting of, alkyl, aryl, cycloalkyl, arylalkyl, heterocyclyl, heteroaryl;
each R⁹ is independently selected from the group consisting of hydrogen, alkyl, aryl, cycloalkyl, arylalkyl, heterocyclyl, heteroaryl;
each R¹⁰ is independently selected from the group consisting of hydrogen, alkyl, aryl, cycloalkyl, arylalkyl, heterocyclyl, heteroaryl;
each Z¹ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z² is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z³ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z⁴ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z⁵ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z⁶ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z⁷ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro; and a pharmaceutically acceptable carrier.

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The independent and dependent claims set out particular and preferred features of the invention. Features from the dependent claims may be combined with features of the independent or other dependent claims as appropriate.

### BRIEF DESCRIPTION OF THE FIGURES

The following description of the figures of specific embodiments of the invention is merely exemplary in nature and is not intended to limit the present teachings, their application or uses.

FIG. 1 represents a graph plotting the percentage of relative light unit measured on PathHunter© cells against the concentration of the compounds (M). To each concentration of the compounds 0.1 μM of NRG-1 was added.

FIG. 2 section A, represents a graph plotting the mean fluorescence measured on PathHunter© cells against the concentration of fluorescent NRG-1 (F-NRG-1) (M). Section B represents a graph plotting the percentage of specific binding to the ERBB4 receptor against the concentration of NRG-1 (M), when competing against 30 nM of F-NRG-1. Section C represents a graph plotting the percentage of specific binding to the ERBB4 receptor against the concentration of compound 1 (M), when competing against F-NRG-1. Section represents a graph plotting the percentage of specific binding to the ERBB4 receptor against the concentration of compound 6 (M), when competing against F-NRG-1.

FIG. 3 represents a graph plotting the luminiscence percentage of light unit (minus PBS) relative to a 100% adenylate kinase (AK) control against the compounds of the invention, 10 μM NRG-1 and 0.1 μM NRG-1.

FIG. 4 represents a graph plotting the fold change in absorbance to a PBS control against 32 μM of the compounds on their own or 32 μM of the compounds combined with 0.1 μM NRG-1.

FIG. 5 represents a graph plotting the mean fluorescence intensity (MFI) (plotted as the fold change to PBS) in iAM cells, against the compounds of the invention, PBS, NRG1 or EGF.

FIG. 6 represents four graphs plotting the mean fluorescence intensity (MFI) (plotted as the fold change to PBS) in EFO-21 cells, against the compounds of the invention, PBS, NRG1 or EGF. Section A corresponds to phospho ERBB1; section B corresponds to phospho ERBB2, section C corresponds to phospho ERBB3 and Section D corresponds to phospho ERBB4.

FIG. 7 represents a graph plotting the luminescence signal of PathHunter® U2OS ERBB2/ERBB3 dimerization cells, against the compounds of the invention, PBS or NRG1.

FIG. 8 section A represents a graph plotting the col1a1 level in human dermal fibroblasts (HDF) (plotted as the fold change to TGFβ), against the compounds of the invention and TGFβ. Section B represents a graph plotting the col3a1 level in HDF (plotted as the fold change to TGFβ), against the compounds of the invention and TGFβ. Section C represents a graph plotting col1a1 level in HDF (plotted as the fold change to TGFβ), against compounds 6 and 7 of the invention and TGFβ. Section D represents a graph plotting col3a1 level in HDF (plotted as the fold change to TGFβ), against compounds 6 and 7 of the invention and TGFβ. Section E represents a graph plotting col1a1 level in HDF (plotted as the fold change to TGFβ), against inactive compounds NA1 and NA2 and TGFβ. Section F represents a graph plotting col3a1 level in HDF, against inactive compounds NA1 and NA2 and TGFβ.

FIG. 9 section A represents a graph plotting the col1a1 level in human atrial cardiac fibroblasts (HCF) (plotted as the fold change to TGFβ), against the compounds of the invention and TGFβ. Section B represents a graph plotting the col3a1 level in HCF (plotted as the fold change to TGFβ), against the compounds of the invention and TGFβ.

FIG. 10 represents a graph plotting the absorbance (solubility) of a dilution of compounds 1, 4, 6 and 7 of the invention, against the concentration of the sample (μM) FIG. 11 represents a graph plotting the % of parent compound remaining of the compound of invention 1, 4, 6 and 7, against time in hours, at 37° C. and pH 7.4.

FIG. 12 represents a graph plotting the % of parent compound remaining of the compound of invention 1, 4, 6 and 7, against time in hours, in human and mouse plasma.

FIG. 13 represents a graph plotting the % of parent compound remaining of the compound of invention 1, 4, 6 and 7, against time in hours, in the presence of human and mouse liver microsomes.

FIG. 14 section A represents a graph plotting the % of positive fibrosis area in mice left ventricular (LV) sections, against control (sham), angiotensin II (AngII), compound 1 of the invention, a combination of AngII and vehicle and a combination of AngII and compound 1 of the invention; this section also shows 5 micrographs of stained mice LV sections. Section B represents a graph plotting the heart-to-body-weight ratio (HW:BW) in mice, against control (sham), angiotensin II (AngII), compound 1 of the invention, a combination of AngII and vehicle and a combination of AngII and compound 1 of the invention. Section C represents a graph plotting the mean cardiomyocyte cross-sectional area (CSA, μm²) of mice, against control (sham), angiotensin II (AngII), compound 1 of the invention, a combination of AngII and vehicle and a combination of AngII and compound 1 of the invention. Section D represents a graph plotting the capillary density of LV sections of mice, against control (sham), angiotensin II (AngII), compound 1 of the invention, a combination of AngII and vehicle and a combination of AngII and compound 1 of the invention.

FIG. 15 represents two stained micrographs of mice hearts that were treated with cumulative dosis of compound 1 of the invention or vehicle; two micrographs of mice livers that were treated with cumulative dosis of compound 1 of the invention or vehicle and two micrographs of mice kidneys that were treated with cumulative dosis of compound 1 of the invention or vehicle FIG. 16 section A represents a graph plotting the percentage of immortalized rat atrial cardiomyocytes death, against a combination of 100 μM H₂O₂ and 10 μM a compound of the invention. Section B represents a graph plotting the percentage of immortalized rat atrial cardiomyocytes death, against a combination of 100 μM H₂O₂ and 4-32 μM of compound 1, 6 or 7 of the invention. Section C represents a graph plotting the percentage of immortalized rat atrial cardiomyocytes death, against a combination of 100 μM H₂O₂ and 4-32 μM of non-active compounds (NA) 1 or 2.

FIG. 17 section A represents a micrograph of rat atrial cardiomyocytes stimulated 24 hours with PBS. Section B represents a micrograph of rat atrial cardiomyocytes stimulated 24 hours with 100 nM angiotensin II (AngII) and 10 μM compound 1 of the invention. Section C represents a micrograph of rat atrial cardiomyocytes stimulated 24 hours with 100 nM AngII and 10 μM compound 2 of the invention. Section D represents a micrograph of rat atrial cardiomyocytes stimulated 24 hours with 100 nM AngII and 10 μM compound 3 of the invention. Section E represents a micrograph of rat atrial cardiomyocytes stimulated 24 hours with 100 nM AngII and 10 μM compound 4 of the invention. Section F represents a micrograph of rat atrial cardiomyocytes stimulated 24 hours with 100 nM AngII and 10 μM compound 5 of the invention. Section G represents a micrograph of rat atrial cardiomyocytes stimulated 24 hours with 100 nM AngII and 10 μM compound 6 of the invention. Section H represents a micrograph of rat atrial cardiomyocytes stimulated 24 hours with 100 nM AngII and 10 μM compound 7 of the invention. Section I represents a micrograph of rat atrial cardiomyocytes stimulated 24 hours with 100 nM AngII and 10 μM compound 8 of the invention. Section J represents a micrograph of rat atrial cardiomyocytes stimulated 24 hours with 100 nM AngII. Section K represents a graph plotting the mean cardiomyocyte cross-sectional area of 20 rat atrial cardiomyocytes, against the compounds of the invention stimulated with 100 nM AngII, PBS and AngII.

FIG. 18 represents a graph plotting the ERBB4/ERBB4 dimer formation in U2OS ERBB4/ERBB4 dimerization cell line, against 10 μM of compound of the invention 1, 10 μM of erlotinib, 10 μM of gefitinib, 32 μM of compound of the invention 1, 32 μM of erlotinib, 32 μM of gefitinib and PBS.

### DETAILED DESCRIPTION OF THE INVENTION

Before the present invention is described, it is to be understood that this invention is not limited to particular processes, methods, and compounds described, as such processes, methods, and compounds may, of course, vary. It is also to be understood that the terminology used herein is not intended to be limiting, since the scope of the present invention will be limited only by the appended claims.

When describing the compounds and processes of the invention, the terms used are to be construed in accordance with the following definitions, unless a context dictates otherwise.

As used in the specification and the appended claims, the singular forms “a”, “an,” and “the” include both singular and plural referents unless the context clearly dictates otherwise. By way of example, “a compound” means one compound or more than one compound.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

As used herein, the term “and/or,” when used in a list of two or more items, means that any one of the listed items can be employed by itself or any combination of two or more of the listed items can be employed. For example, if a list is described as comprising group A, B, and/or C, the list can comprise A alone; B alone; C alone; A and B in combination; A and C in combination, B and C in combination; or A, B, and C in combination.

The recitation of numerical ranges by endpoints includes all integer numbers and, where appropriate, fractions subsumed within that range (e.g. 1 to 5 can include 1, 2, 3, 4 when referring to, for example, a number of elements, and can also include 1.5, 2, 2.75 and 3.80, when referring to, for example, measurements). The recitation of end points also includes the end point values themselves (e.g. from 1.0 to 5.0 includes both 1.0 and 5.0). Any numerical range recited herein is intended to include all sub-ranges subsumed therein.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiments but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments combinations of features of different embodiments are meant to be within the scope of the invention, and form different embodiments as would be understood by those in the art. For example, in the following claims, any of the claimed embodiments can be used in any combination.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention.

When describing the present invention, the terms used are to be construed in accordance with the following definitions, unless a context dictates otherwise.

The terms described above and others used in the specification are well understood to those in the art.

Whenever the term “substituted” is used herein, it is meant to indicate that one or more hydrogen atoms on the atom indicated in the expression using “substituted” is replaced with a selection from the indicated group, provided that the indicated atom's normal valence is not exceeded, and that the substitution results in a chemically stable compound, i.e. a compound that is sufficiently robust to survive isolation from a reaction mixture.

Where groups can be substituted, such groups may be substituted with one or more, and preferably one, two or three substituents. Preferred substituents may be selected from but not limited to, for example, the group comprising halo, hydroxyl, alkyl, alkoxy, trifluoromethyl, trifluoromethoxy, cycloalkyl, aryl, arylalkyl, heterocyclyl, heteroaryl, cyano, amino, nitro, carboxyl, and mono- or dialkylamino.

The term “halo” or “halogen” as a group or part of a group is generic for fluoro, chloro, bromo, iodo.

The term “hydroxyl” or “hydroxy” as used herein refers to the group —OH.

The term “cyano” as used herein refers to the group —C≡N.

The term “amino” as used herein refers to the —NH₂group.

The term “nitro” as used herein refers to the —NO₂ group.

The term “carboxy” or “carboxyl” or “hydroxycarbonyl” as used herein refers to the group —CO₂H.

The term “aminocarbonyl” as used herein refers to the group —CONH₂.

The term “alkyl”, as a group or part of a group, refers to a hydrocarbyl group of formula —CₙH₂ₙ₊₁ wherein n is a number greater than or equal to 1. Alkyl groups may be linear or branched and may be substituted as indicated herein. Generally, alkyl groups of this invention comprise from 1 to 6 carbon atoms, preferably from 1 to 5 carbon atoms, preferably from 1 to 4 carbon atoms, more preferably from 1 to 3 carbon atoms, still more preferably 1 to 2 carbon atoms. When a subscript is used herein following a carbon atom, the subscript refers to the number of carbon atoms that the named group may contain. For example, “C₁₋₆alkyl” includes all linear or branched alkyl groups with between 1 and 6 carbon atoms, and thus includes methyl, ethyl, n-propyl, i-propyl, butyl and its isomers (e.g. n-butyl, i-butyl and t-butyl); pentyl and its isomers, hexyl and its isomers. For example, “C₁₋₅alkyl” includes all includes all linear or branched alkyl groups with between 1 and 5 carbon atoms, and thus includes methyl, ethyl, n-propyl, i-propyl, butyl and its isomers (e.g. n-butyl, i-butyl and t-butyl); pentyl and its isomers. For example, “C₁₋₄alkyl” includes all linear or branched alkyl groups with between 1 and 4 carbon atoms, and thus includes methyl, ethyl, n-propyl, i-propyl, butyl and its isomers (e.g. n-butyl, i-butyl and t-butyl). For example “C₁₋₃alkyl” includes all linear or branched alkyl groups with between 1 and 3 carbon atoms, and thus includes methyl, ethyl, n-propyl, i-propyl.

When the term “alkyl” is used as a suffix following another term, as in “hydroxyalkyl,” this is intended to refer to an alkyl group, as defined above, being substituted with one or two (preferably one) substituent(s) selected from the other, specifically-named group, also as defined herein. The term “hydroxyalkyl” therefore refers to a —R^{a}—OH group wherein R^{a} is alkylene as defined herein.

The term “haloalkyl” as a group or part of a group, refers to a alkyl group having the meaning as defined above wherein one, two, or three hydrogen atoms are each replaced with a halogen as defined herein. Non-limiting examples of such haloalkyl groups include chloromethyl, 1-bromoethyl, fluoromethyl, difluoromethyl, trifluoromethyl, 1,1,1-trifluoroethyl, trichloromethyl, tribromomethyl, and the like.

The term “trifluoromethyl” as used herein refers to the group —CF₃.

The term “difluoromethyl” as used herein refers to the group —CHF₂.

The term “trifluoromethoxy” as used herein refers to the group —OCF₃.

The term “difluoromethoxy” as used herein refers to the group —OCHF₂.

The term “alkoxy” or “alkyloxy”, as a group or part of a group, refers to a group having the formula —OR^{b} wherein R^{b} is alkyl as defined herein above. Non-limiting examples of suitable alkoxy include methoxy, ethoxy, propoxy, isopropoxy, butoxy, isobutoxy, sec-butoxy, tert-butoxy, pentyloxy and hexyloxy.

The term “cycloalkyl”, as a group or part of a group, refers to a cyclic alkyl group, that is a monovalent, saturated, hydrocarbyl group having 1 or more cyclic structure, and comprising from 3 to 12 carbon atoms, more preferably from 3 to 9 carbon atoms, more preferably from 3 to 7 carbon atoms; more preferably from 3 to 6 carbon atoms. Cycloalkyl includes all saturated hydrocarbon groups containing 1 or more rings, including monocyclic or bicyclic groups. The further rings of multi-ring cycloalkyls may be either fused, bridged and/or joined through one or more spiro atoms. When a subscript is used herein following a carbon atom, the subscript refers to the number of carbon atoms that the named group may contain. For example, the term “C₃₋₈cycloalkyl”, a cyclic alkyl group comprising from 3 to 8 carbon atoms. For example, the term “C₃₋₆cycloalkyl”, a cyclic alkyl group comprising from 3 to 6 carbon atoms. Examples of C₃₋₁₂cycloalkyl groups include but are not limited to cyclopropyl, cyclobutyl, cyclopentyl, cyclohexyl, cycloheptyl, cyclooctyl, bicycle[2.2.1]heptan-2yl, (1S,4R)-norbornan-2-yl, (1R,4R)-norbornan-2-yl, (1S,4S)-norbornan-2-yl, (1R,4S)-norbornan-2-yl, 1-adamantyl.

The term “cycloalkyloxy”, as a group or part of a group, refers to a group having the formula —OR^{f} wherein R^{f} is cycloalkyl as defined herein above.

The term “alkenyl” as a group or part of a group, refers to an unsaturated hydrocarbyl group, which may be linear, or branched, comprising one or more carbon-carbon double bonds. When a subscript is used herein following a carbon atom, the subscript refers to the number of carbon atoms that the named group may contain. For example, the term “C₂₋₆alkenyl” refers to an unsaturated hydrocarbyl group, which may be linear, or branched comprising one or more carbon-carbon double bonds and comprising from 2 to 6 carbon atoms. For example, C₂₋₄alkenyl includes all linear, or branched alkenyl groups having 2 to 4 carbon atoms. Examples of C₂₋₆alkenyl groups are ethenyl, 2-propenyl, 2-butenyl, 3-butenyl, 2-pentenyl and its isomers, 2-hexenyl and its isomers, 2,4-pentadienyl, and the like.

The term “aryl”, as a group or part of a group, refers to a polyunsaturated, aromatic hydrocarbyl group having a single ring (i.e. phenyl) or multiple aromatic rings fused together (e.g. naphthyl), or linked covalently, typically comprising 6 to 12 carbon atoms; wherein at least one ring is aromatic, preferably comprising 6 to 10 carbon atoms, wherein at least one ring is aromatic. The aromatic ring may optionally include one to two additional rings (either cycloalkyl, heterocyclyl or heteroaryl) fused thereto. Examples of suitable aryl include C₆₋₁₂aryl, preferably C₆₋₁₀aryl, more preferably C₆₋₈aryl. Non-limiting examples of aryl comprise phenyl, biphenylyl, biphenylenyl, or 1- or 2-naphthanelyl; 5- or 6-tetralinyl, 1-, 2-, 3-, 4-, 5-, 6-, 7- or 8-azulenyl, 4-, 5-, 6 or 7-indenyl, 4- or 5-indanyl, 5-, 6-, 7- or 8-tetrahydronaphthyl, 1,2,3,4-tetrahydronaphthyl, and 1,4-dihydronaphthyl; 1-, 2-, 3-, 4- or 5-pyrenyl. A “substituted aryl” refers to an aryl group having one or more substituent(s) (for example 1, 2 or 3 substituent(s), or 1 to 2 substituent(s)), at any available point of attachment.

The term “aryloxy”, as a group or part of a group, refers to a group having the formula —OR^{g} wherein R^{g} is aryl as defined herein above.

The term “arylalkyl”, as a group or part of a group, means a alkyl as defined herein, wherein at least one hydrogen atom is replaced by at least one aryl as defined herein. Non-limiting examples of arylalkyl group include benzyl, phenethyl, dibenzylmethyl, methylphenylmethyl, 3-(2-naphthyl)-butyl, and the like.

The terms “heterocyclyl” or “heterocycloalkyl” or “heterocyclo”, as a group or part of a group, refer to non-aromatic, fully saturated or partially unsaturated cyclic groups (for example, 3 to 7 member monocyclic, 7 to 11 member bicyclic, or comprising a total of 3 to 10 ring atoms) which have at least one heteroatom in at least one carbon atom-containing ring; wherein said ring may be fused to an aryl, cycloalkyl, heteroaryl or heterocyclyl ring. Each ring of the heterocyclyl group containing a heteroatom may have 1, 2, 3 or 4 heteroatoms selected from N, O and/or S, where the N and S heteroatoms may optionally be oxidized and the N heteroatoms may optionally be quaternized, and wherein at least one carbon atom of heterocyclyl can be oxidized to form at least one C═O. The heterocyclic group may be attached at any heteroatom or carbon atom of the ring or ring system, where valence allows. The rings of multi-ring heterocycles may be fused, bridged and/or joined through one or more spiro atoms.

Non limiting exemplary heterocyclic groups include aziridinyl, oxiranyl, thiiranyl, piperidinyl, azetidinyl, oxetanyl, pyrrolidinyl, thietanyl, 2-imidazolinyl, pyrazolidinyl imidazolidinyl, isoxazolinyl, oxazolidinyl, isoxazolidinyl, thiazolidinyl, isothiazolidinyl, piperidinyl, succinimidyl, 3H-indolyl, indolinyl, chromanyl (also known as 3,4-dihydrobenzo[b]pyranyl), isoindolinyl, 2H-pyrrolyl, 1-pyrrolinyl, 2-pyrrolinyl, 3-pyrrolinyl, 4H-quinolizinyl, 2-oxopiperazinyl, piperazinyl, homopiperazinyl, 2-pyrazolinyl, 3-pyrazolinyl, tetrahydro-2H-pyranyl, 2H-pyranyl, 4H-pyranyl, 3,4-dihydro-2H-pyranyl, 3-dioxolanyl, 1,4-dioxanyl, 2,5-dioximidazolidinyl, 2-oxopiperidinyl, 2-oxopyrrolodinyl, indolinyl, tetrahydropyranyl, tetrahydrofuranyl, tetrahydrothiophenyl, tetrahydroquinolinyl, tetrahydroisoquinolin-1-yl, tetrahydroisoquinolin-2-yl, tetrahydroisoquinolin-3-yl, tetrahydroisoquinolin-4-yl, thiomorpholin-4-yl, thiomorpholin-4-ylsulfoxide, thiomorpholin-4-ylsulfone, 1,3-dioxolanyl, 1,4-oxathianyl, 1,4-dithianyl, 1,3,5-trioxanyl, 1H-pyrrolizinyl, tetrahydro-1,1-dioxothiophenyl, N-formylpiperazinyl, and morpholin-4-yl. The term “aziridinyl” as used herein includes aziridin-1-yl and aziridin-2-yl. The term “oxyranyl” as used herein includes oxyranyl-2-yl.

The term “thiiranyl” as used herein includes thiiran-2-yl. The term “azetidinyl” as used herein includes azetidin-1-yl, azetidin-2-yl and azetidin-3-yl. The term “oxetanyl” as used herein includes oxetan-2-yl and oxetan-3-yl. The term “thietanyl” as used herein includes thietan-2-yl and thietan-3-yl. The term “pyrrolidinyl” as used herein includes pyrrolidin-1-yl, pyrrolidin-2-yl and pyrrolidin-3-yl. The term “tetrahydrofuranyl” as used herein includes tetrahydrofuran-2-yl and tetrahydrofuran-3-yl. The term “tetrahydrothiophenyl” as used herein includes tetrahydrothiophen-2-yl and tetrahydrothiophen-3-yl. The term “succinimidyl” as used herein includes succinimid-1-yl and succininmid-3-yl. The term “dihydropyrrolyl” as used herein includes 2,3-dihydropyrrol-1-yl, 2,3-dihydro-1H-pyrrol-2-yl, 2,3-dihydro-1H-pyrrol-3-yl, 2,5-dihydropyrrol-1-yl, 2,5-dihydro-1H-pyrrol-3-yl and 2,5-dihydropyrrol-5-yl. The term “2H-pyrrolyl” as used herein includes 2H-pyrrol-2-yl, 2H-pyrrol-3-yl, 2H-pyrrol-4-yl and 2H-pyrrol-5-yl. The term “3H-pyrrolyl” as used herein includes 3H-pyrrol-2-yl, 3H-pyrrol-3-yl, 3H-pyrrol-4-yl and 3H-pyrrol-5-yl. The term “dihydrofuranyl” as used herein includes 2,3-dihydrofuran-2-yl, 2,3-dihydrofuran-3-yl, 2,3-dihydrofuran-4-yl, 2,3-dihydrofuran-5-yl, 2,5-dihydrofuran-2-yl, 2,5-dihydrofuran-3-yl, 2,5-dihydrofuran-4-yl and 2,5-dihydrofuran-5-yl. The term “dihydrothiophenyl” as used herein includes 2,3-dihydrothiophen-2-yl, 2,3-dihydrothiophen-3-yl, 2,3-dihydrothiophen-4-yl, 2,3-dihydrothiophen-5-yl, 2,5-di hydrothiophen-2-yl, 2,5-dihydrothiophen-3-yl, 2,5-dihydrothiophen-4-yl and 2,5-dihydrothiophen-5-yl. The term “imidazolidinyl” as used herein includes imidazolidin-1-yl, imidazolidin-2-yl and imidazolidin-4-yl. The term “pyrazolidinyl” as used herein includes pyrazolidin-1-yl, pyrazolidin-3-yl and pyrazolidin-4-yl. The term “imidazolinyl” as used herein includes imidazolin-1-yl, imidazolin-2-yl, imidazolin-4-yl and imidazolin-5-yl. The term “pyrazolinyl” as used herein includes 1-pyrazolin-3-yl, 1-pyrazolin-4-yl, 2-pyrazolin-1-yl, 2-pyrazolin-3-yl, 2-pyrazolin-4-yl, 2-pyrazolin-5-yl, 3-pyrazolin-1-yl, 3-pyrazolin-2-yl, 3-pyrazolin-3-yl, 3-pyrazolin-4-yl and 3-pyrazolin-5-yl. The term “dioxolanyl” also known as “1,3-dioxolanyl” as used herein includes dioxolan-2-yl, dioxolan-4-yl and dioxolan-5-yl. The term “dioxolyl” also known as “1,3-dioxolyl” as used herein includes dioxol-2-yl, dioxol-4-yl and dioxol-5-yl. The term “oxazolidinyl” as used herein includes oxazolidin-2-yl, oxazolidin-3-yl, oxazolidin-4-yl and oxazolidin-5-yl. The term “isoxazolidinyl” as used herein includes isoxazolidin-2-yl, isoxazolidin-3-yl, isoxazolidin-4-yl and isoxazolidin-5-yl. The term “oxazolinyl” as used herein includes 2-oxazolinyl-2-yl, 2-oxazolinyl-4-yl, 2-oxazolinyl-5-yl, 3-oxazolinyl-2-yl, 3-oxazolinyl-4-yl, 3-oxazolinyl-5-yl, 4 oxazolinyl-2-yl, 4-oxazolinyl-3-yl, 4-oxazolinyl-4-yl and 4-oxazolinyl-5-yl. The term “isoxazolinyl” as used herein includes 2-isoxazolinyl-3-yl, 2-isoxazolinyl-4-yl, 2-isoxazolinyl-5-yl, 3-isoxazolinyl-3-yl, 3-isoxazolinyl-4-yl, 3-isoxazolinyl-5-yl, 4-isoxazolinyl-2-yl, 4-isoxazolinyl-3-yl, 4-isoxazolinyl-4-yl and 4-isoxazolinyl-5-yl. The term “thiazolidinyl” as used herein includes thiazolidin-2-yl, thiazolidin-3-yl, thiazolidin-4-yl and thiazolidin-5-yl. The term “isothiazolidinyl” as used herein includes isothiazolidin-2-yl, isothiazolidin-3-yl, isothiazolidin-4-yl and isothiazolidin-5-yl. The term “chromanyl” as used herein includes chroman-2-yl, chroman-3-yl, chroman-4-yl, chroman-5-yl, chroman-6-yl, chroman-7-yl and chroman-8-yl. The term “thiazolinyl” as used herein includes 2-thiazolinyl-2-yl, 2-thiazolinyl-4-yl, 2-thiazolinyl-5-yl, 3-thiazolinyl-2-yl, 3-thiazolinyl-4-yl, 3-thiazolinyl-5-yl, 4-thiazolinyl-2-yl, 4-thiazolinyl-3-yl, 4-thiazolinyl-4-yl and 4-thiazolinyl-5-yl. The term “isothiazolinyl” as used herein includes 2-isothiazolinyl-3-yl, 2-isothiazolinyl-4-yl, 2-isothiazolinyl-5-yl, 3-isothiazolinyl-3-yl, 3-isothiazolinyl-4-yl, 3-isothiazolinyl-5-yl, 4-isothiazolinyl-2-yl, 4-isothiazolinyl-3-yl, 4-isothiazolinyl-4-yl and 4-isothiazolinyl-5-yl. The term “piperidyl” also known as “piperidinyl” as used herein includes piperid-1-yl, piperid-2-yl, piperid-3-yl and piperid-4-yl. The term “dihydropyridinyl” as used herein includes 1,2-dihydropyridin-1-yl, 1,2-dihydropyridin-2-yl, 1,2-dihydropyridin-3-yl, 1,2-dihydropyridin-4-yl, 1,2-dihydropyridin-5-yl, 1,2-dihydropyridin-6-yl, 1,4-dihydropyridin-1-yl, 1,4-dihydropyridin-2-yl, 1,4-dihydropyridin-3-yl, 1,4-dihydropyridin-4-yl, 2,3-dihydropyridin-2-yl, 2,3-dihydropyridin-3-yl, 2,3-dihydropyridin-4-yl, 2,3-dihydropyridin-5-yl, 2,3-dihydropyridin-6-yl, 2,5-dihydropyridin-2-yl, 2,5-dihydropyridin-3-yl, 2,5-dihydropyridin-4-yl, 2,5-dihydropyridin-5-yl, 2,5-dihydropyridin-6-yl, 3,4-dihydropyridin-2-yl, 3,4-dihydropyridin-3-yl, 3,4-dihydropyridin-4-yl, 3,4-dihydropyridin-5-yl and 3,4-dihydropyridin-6-yl.

The term “tetrahydropyridinyl” as used herein includes 1,2,3,4-tetrahydropyridin-1-yl, 1,2,3,4-tetrahydropyridin-2-yl, 1,2,3,4-tetrahydropyridin-3-yl, 1,2,3,4-tetrahydropyridin-4-yl, 1,2,3,4-tetrahydropyridin-5-yl, 1,2,3,4-tetrahydropyridin-6-yl, 1,2,3,6-tetrahydropyridin-1-yl, 1,2,3,6-tetrahydropyridin-2-yl, 1,2,3,6-tetrahydropyridin-3-yl, 1,2,3,6-tetrahydropyridin-4-yl, 1,2,3,6-tetrahydropyridin-5-yl, 1,2,3,6-tetrahydropyridin-6-yl, 2,3,4,5-tetrahydropyridin-2-yl, 2,3,4,5-tetrahydropyridin-3-yl, 2,3,4,5-tetrahydropyridin-3-yl, 2,3,4,5-tetrahydropyridin-4-yl, 2,3,4,5-tetrahydropyridin-5-yl and 2,3,4,5-tetrahydropyridin-6-yl. The term “tetrahydropyranyl” also known as “oxanyl” or “tetrahydro-2H-pyranyl”, as used herein includes tetrahydropyran-2-yl, tetrahydropyran-3-yl and tetrahydropyran-4-yl. The term “2H-pyranyl” as used herein includes 2H-pyran-2-yl, 2H-pyran-3-yl, 2H-pyran-4-yl, 2H-pyran-5-yl and 2H-pyran-6-yl. The term “4H-pyranyl” as used herein includes 4H-pyran-2-yl, 4H-pyran-3-yl and 4H-pyran-4-yl. The term “3,4-dihydro-2H-pyranyl” as used herein includes 3,4-dihydro-2H-pyran-2-yl, 3,4-dihydro-2H-pyran-3-yl, 3,4-dihydro-2H-pyran-4-yl, 3,4-dihydro-2H-pyran-5-yl and 3,4-dihydro-2H-pyran-6-yl. The term “3,6-dihydro-2H-pyranyl” as used herein includes 3,6-dihydro-2H-pyran-2-yl, 3,6-dihydro-2H-pyran-3-yl, 3,6-dihydro-2H-pyran-4-yl, 3,6-dihydro-2H-pyran-5-yl and 3,6-dihydro-2H-pyran-6-yl. The term “tetrahydrothiophenyl”, as used herein includes tetrahydrothiophen-2-yl, tetrahydrothiophenyl-3-yl and tetrahydrothiophenyl-4-yl. The term “2H-thiopyranyl” as used herein includes 2H-thiopyran-2-yl, 2H-thiopyran-3-yl, 2H-thiopyran-4-yl, 2H-thiopyran-5-yl and 2H-thiopyran-6-yl. The term “4H-thiopyranyl” as used herein includes 4H-thiopyran-2-yl, 4H-thiopyran-3-yl and 4H-thiopyran-4-yl. The term “3,4-dihydro-2H-thiopyranyl” as used herein includes 3,4-dihydro-2H-thiopyran-2-yl, 3,4-dihydro-2H-thiopyran-3-yl, 3,4-dihydro-2H-thiopyran-4-yl, 3,4-dihydro-2H-thiopyran-5-yl and 3,4-dihydro-2H-thiopyran-6-yl. The term “3,6-dihydro-2H-thiopyranyl” as used herein includes 3,6-dihydro-2H-thiopyran-2-yl, 3,6-dihydro-2H-thiopyran-3-yl, 3,6-dihydro-2H-thiopyran-4-yl, 3,6-dihydro-2H-thiopyran-5-yl and 3,6-dihydro-2H-thiopyran-6-yl. The term “piperazinyl” also known as “piperazidinyl” as used herein includes piperazin-1-yl and piperazin-2-yl. The term “morpholinyl” as used herein includes morpholin-2-yl, morpholin-3-yl and morpholin-4-yl. The term “thiomorpholinyl” as used herein includes thiomorpholin-2-yl, thiomorpholin-3-yl and thiomorpholin-4-yl. The term “dioxanyl” as used herein includes 1,2-dioxan-3-yl, 1,2-dioxan-4-yl, 1,3-dioxan-2-yl, 1,3-dioxan-4-yl, 1,3-dioxan-5-yl and 1,4-dioxan-2-yl.

The term “dithianyl” as used herein includes 1,2-dithian-3-yl, 1,2-dithian-4-yl, 1,3-dithian-2-yl, 1,3-dithian-4-yl, 1,3-dithian-5-yl and 1,4-dithian-2-yl. The term “oxathianyl” as used herein includes oxathian-2-yl and oxathian-3-yl. The term “trioxanyl” as used herein includes 1,2,3-trioxan-4-yl, 1,2,3-trioxay-5-yl, 1,2,4-trioxay-3-yl, 1,2,4-trioxay-5-yl, 1,2,4-trioxay-6-yl and 1,3,4-trioxay-2-yl.

The term “azepanyl” as used herein includes azepan-1-yl, azepan-2-yl, azepan-1-yl, azepan-3-yl and azepan-4-yl. The term “homopiperazinyl” as used herein includes homopiperazin-1-yl, homopiperazin-2-yl, homopiperazin-3-yl and homopiperazin-4-yl. The term “indolinyl” as used herein includes indolin-1-yl, indolin-2-yl, indolin-3-yl, indolin-4-yl, indolin-5-yl, indolin-6-yl, and indolin-7-yl. The term “quinolizinyl” as used herein includes quinolizidin-1-yl, quinolizidin-2-yl, quinolizidin-3-yl and quinolizidin-4-yl. The term “isoindolinyl” as used herein includes isoindolin-1-yl, isoindolin-2-yl, isoindolin-3-yl, isoindolin-4-yl, isoindolin-5-yl, isoindolin-6-yl, and isoindolin-7-yl. The term “3H-indolyl” as used herein includes 3H-indol-2-yl, 3H-indol-3-yl, 3H-indol-4-yl, 3H-indol-5-yl, 3H-indol-6-yl, and 3H-indol-7-yl. The term “quinolizinyl” as used herein includes quinolizidin-1-yl, quinolizidin-2-yl, quinolizidin-3-yl and quinolizidin-4-yl. The term “quinolizinyl” as used herein includes quinolizidin-1-yl, quinolizidin-2-yl, quinolizidin-3-yl and quinolizidin-4-yl. The term “tetrahydroquinolinyl” as used herein includes tetrahydroquinolin-1-yl, tetrahydroquinolin-2-yl, tetrahydroquinolin-3-yl, tetrahydroquinolin-4-yl, tetrahydroquinolin-5-yl, tetrahydroquinolin-6-yl, tetrahydroquinolin-7-yl and tetrahydroquinolin-8-yl. The term “tetrahydroisoquinolinyl” as used herein includes tetrahydroisoquinolin-1-yl, tetrahydroisoquinolin-2-yl, tetrahydroisoquinolin-3-yl, tetrahydroisoquinolin-4-yl, tetrahydroisoquinolin-5-yl, tetrahydroisoquinolin-6-yl, tetrahydroisoquinolin-7-yl and tetrahydroisoquinolin-8-yl. The term “1H-pyrrolizine” as used herein includes 1H-pyrrolizin-1-yl, 1H-pyrrolizin-2-yl, 1H-pyrrolizin-3-yl, 1H-pyrrolizin-5-yl, 1H-pyrrolizin-6-yl and 1H-pyrrolizin-7-yl. The term “3H-pyrrolizine” as used herein includes 3H-pyrrolizin-1-yl, 3H-pyrrolizin-2-yl, 3H-pyrrolizin-3-yl, 3H-pyrrolizin-5-yl, 3H-pyrrolizin-6-yl and 3H pyrrolizin-7-yl.

The term “heterocyclyloxy”, as a group or part of a group, refers to a group having the formula —O—Rⁱ wherein Rⁱ is heterocyclyl as defined herein above.

The term “heteroaryl” as a group or part of a group, refers but is not limited to 5 to 12 carbon-atom aromatic rings or ring systems containing 1 or 2 rings which can be fused together or linked covalently, typically containing 5 to 6 atoms; at least one of which is aromatic in which one or more carbon atoms in one or more of these rings can be replaced by N, O and/or S atoms where the N and S heteroatoms may optionally be oxidized and the N heteroatoms may optionally be quaternized, and wherein at least one carbon atom of said heteroaryl can be oxidized to form at least one C═O. Such rings may be fused to an aryl, cycloalkyl, heteroaryl or heterocyclyl ring. Non-limiting examples of such heteroaryl, include: pyrrolyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyraziyl, pyridazinyl, oxazinyl, dioxinyl, thiazinyl, triazinyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, benzo[d]oxazol-2(3H)-one, 2,3-dihydro-benzofuranyl, thienopyridinyl, purinyl, imidazo[1,2-a]pyridinyl, 6-oxo-pyridazin-1(6H)-yl, 2-oxopyridin-1(2H)-yl, 6-oxo-pyridazin-1(6H)-yl, 2-oxopyridin-1(2H)-yl, 1,3-benzodioxolyl, quinolinyl, isoquinolinyl, cinnolinyl, quinazolinyl, quinoxalinyl; preferably said heteroaryl group is selected from the group consisting of pyridyl, 1,3-benzodioxolyl, benzo[d]oxazol-2(3H)-one, 2,3-dihydro-benzofuranyl, pyrazinyl, pyrazolyl, pyrrolyl, isoxazolyl, thiophenyl, imidazolyl, benzimidazolyl, pyrimidinyl, triazolyl and thiazolyl.

The term “pyrrolyl” (also called azolyl) as used herein includes pyrrol-1-yl, pyrrol-2-yl and pyrrol-3-yl. The term “furanyl” (also called “furyl”) as used herein includes furan-2-yl and furan-3-yl (also called furan-2-yl and furan-3-yl). The term “thiophenyl” (also called “thienyl”) as used herein includes thiophen-2-yl and thiophen-3-yl (also called thien-2-yl and thien-3-yl). The term “pyrazolyl” (also called 1H-pyrazolyl and 1,2-diazolyl) as used herein includes pyrazol-1-yl, pyrazol-3-yl, pyrazol-4-yl and pyrazol-5-yl. The term “imidazolyl” as used herein includes imidazol-1-yl, imidazol-2-yl, imidazol-4-yl and imidazol-5-yl. The term “oxazolyl” (also called 1,3-oxazolyl) as used herein includes oxazol-2-yl, oxazol-4-yl and oxazol-5-yl. The term “isoxazolyl” (also called 1,2-oxazolyl), as used herein includes isoxazol-3-yl, isoxazol-4-yl, and isoxazol-5-yl. The term “thiazolyl” (also called 1,3-thiazolyl), as used herein includes thiazol-2-yl, thiazol-4-yl and thiazol-5-yl (also called 2-thiazolyl, 4-thiazolyl and 5-thiazolyl). The term “isothiazolyl” (also called 1,2-thiazolyl) as used herein includes isothiazol-3-yl, isothiazol-4-yl, and isothiazol-5-yl. The term “triazolyl” as used herein includes 1H-triazolyl and 4H-1,2,4-triazolyl, “1H-triazolyl” includes 1H-1,2,3-triazol-1-yl, 1H-1,2,3-triazol-4-yl, 1H-1,2,3-triazol-5-yl, 1H-1,2,4-triazol-1-yl, 1H-1,2,4-triazol-3-yl and 1H-1,2,4-triazol-5-yl. “4H-1,2,4-triazolyl” includes 4H-1,2,4-triazol-4-yl, and 4H-1,2,4-triazol-3-yl. The term “oxadiazolyl” as used herein includes 1,2,3-oxadiazol-4-yl, 1,2,3-oxadiazol-5-yl, 1,2,4-oxadiazol-3-yl, 1,2,4-oxadiazol-5-yl, 1,2,5-oxadiazol-3-yl and 1,3,4 oxadiazol-2-yl. The term “thiadiazolyl” as used herein includes 1,2,3-thiadiazol-4-yl, 1,2,3-thiadiazol-5-yl, 1,2,4-thiadiazol-3-yl, 1,2,4-thiadiazol-5-yl, 1,2,5-thiadiazol-3-yl (also called furazan-3-yl) and 1,3,4-thiadiazol-2-yl. The term “tetrazolyl” as used herein includes 1H-tetrazol-1-yl, 1H-tetrazol-5-yl, 2H-tetrazol-2-yl, and 2H-tetrazol-5-yl. The term “oxatriazolyl” as used herein includes 1,2,3,4-oxatriazol-5-yl and 1,2,3,5-oxatriazol-4-yl. The term “thiatriazolyl” as used herein includes 1,2,3,4-thiatriazol-5-yl and 1,2,3,5-thiatriazol-4-yl. The term “pyridinyl” (also called “pyridyl”) as used herein includes pyridin-2-yl, pyridin-3-yl and pyridin-4-yl (also called 2-pyridyl, 3-pyridyl and 4-pyridyl). The term “pyrimidyl” as used herein includes pyrimid-2-yl, pyrimid-4-yl, pyrimid-5-yl and pyrimid-6-yl. The term “pyrazinyl” as used herein includes pyrazin-2-yl and pyrazin-3-yl. The term “pyridazinyl” as used herein includes pyridazin-3-yl and pyridazin-4-yl. The term “oxazinyl” (also called “1,4-oxazinyl”) as used herein includes 1,4-oxazin-4-yl and 1,4-oxazin-5-yl. The term “dioxinyl” (also called “1,4-dioxinyl”) as used herein includes 1,4-dioxin-2-yl and 1,4-dioxin-3-yl. The term “thiazinyl” (also called “1,4-thiazinyl”) as used herein includes 1,4-thiazin-2-yl, 1,4-thiazin-3-yl, 1,4-thiazin-4-yl, 1,4-thiazin-5-yl and 1,4-thiazin-6-yl. The term “triazinyl” as used herein includes 1,3,5-triazin-2-yl, 1,2,4-triazin-3-yl, 1,2,4-triazin-5-yl, 1,2,4 triazin-6-yl, 1,2,3-triazin-4-yl and 1,2,3-triazin-5-yl. The term “imidazo[2,1-b][1,3]thiazolyl” as used herein includes imidazo[2,1-b][1,3]thiazoi-2-yl, imidazo[2,1-b][1,3]thiazol-3-yl, imidazo[2,1-b][1,3]thiazol-5-yl and imidazo[2,1-b][1,3]thiazol-6-yl. The term “thieno[3,2-b]furanyl” as used herein includes thieno[3,2-b]furan-2-yl, thieno[3,2-b]furan-3-yl, thieno[3,2-b]furan-4-yl, and thieno[3,2-b]furan-5-yl. The term “thieno[3,2-b]thiophenyl” as used herein includes thieno[3,2-b]thien-2-yl, thieno[3,2-b]thien-3-yl, thieno[3,2-b]thien-5-yl and thieno[3,2-b]thien-6-yl. The term “thieno[2,3-d][1,3]thiazolyl” as used herein includes thieno[2,3-d][1,3]thiazol-2-yl, thieno[2,3-d][1,3]thiazol-5-yl and thieno[2,3-d][1,3]thiazol-6-yl. The term “thieno[2,3-d]imidazolyl” as used herein includes thieno[2,3-d]imidazol-2-yl, thieno[2,3-d]imidazol-4-yl and thieno[2,3-d]imidazol-5-yl. The term “tetrazolo[1,5-a]pyridinyl” as used herein includes tetrazolo[1,5-a]pyridine-5-yl, tetrazolo[1,5-a]pyridine-6-yl, tetrazolo[1,5-a]pyridine-7-yl, and tetrazolo[1,5-a]pyridine-8-yl. The term “indolyl” as used herein includes indol-1-yl, indol-2-yl, indol-3-yl, -indol-4-yl, indol-5-yl, indol-6-yl and indol-7-yl. The term “indolizinyl” as used herein includes indolizin-1-yl, indolizin-2-yl, indolizin-3-yl, indolizin-5-yl, indolizin-6-yl, indolizin-7-yl, and indolizin-8-yl. The term “isoindolyl” as used herein includes isoindol-1-yl, isoindol-2-yl, isoindol-3-yl, isoindol-4-yl, isoindol-5-yl, isoindol-6-yl and isoindol-7-yl. The term “benzofuranyl” (also called benzo[b]furanyl) as used herein includes benzofuran-2-yl, benzofuran-3-yl, benzofuran-4-yl, benzofuran-5-yl, benzofuran-6-yl and benzofuran-7-yl. The term “isobenzofuranyl” (also called benzo[c]furanyl) as used herein includes isobenzofuran-1-yl, isobenzofuran-3-yl, isobenzofuran-4-yl, isobenzofuran-5-yl, isobenzofuran-6-yl and isobenzofuran-7-yl. The term “benzothiophenyl” (also called benzo[b]thienyl) as used herein includes 2-benzo[b]thiophenyl, 3-benzo[b]thiophenyl, 4-benzo[b]thiophenyl, 5-benzo[b]thiophenyl, 6-benzo[b]thiophenyl and -7-benzo[b]thiophenyl (also called benzothien-2-yl, benzothien-3-yl, benzothien-4-yl, benzothien-5-yl, benzothien-6-yl and benzothien-7-yl). The term “isobenzothiophenyl” (also called benzo[c]thienyl) as used herein includes isobenzothien-1-yl, isobenzothien-3-yl, isobenzothien-4-yl, isobenzothien-5-yl, isobenzothien-6-yl and isobenzothien-7-yl. The term “indazolyl” (also called 1H-indazolyl or 2-azaindolyl) as used herein includes 1H-indazol-1-yl, 1H-indazol-3-yl, 1H-indazol-4-yl, 1H-indazol-5-yl, 1H-indazol-6-yl, 1H-indazol-7-yl, 2H-indazol-2-yl, 2H-indazol-3-yl, 2H-indazol-4-yl, 2H-indazol-5-yl, 2H-indazol-6-yl, and 2H-indazol-7-yl. The term “benzimidazolyl” as used herein includes benzimidazol-1-yl, benzimidazol-2-yl, benzimidazol-4-yl, benzimidazol-5-yl, benzimidazol-6-yl and benzimidazol-7-yl. The term “1,3-benzoxazolyl” as used herein includes 1,3-benzoxazol-2-yl, 1,3-benzoxazol-4-yl, 1,3-benzoxazol-5-yl, 1,3-benzoxazol-6-yl and 1,3-benzoxazol-7-yl. The term “1,2-benzisoxazolyl” as used herein includes 1,2-benzisoxazol-3-yl, 1,2-benzisoxazol-4-yl, 1,2-benzisoxazol-5-yl, 1,2-benzisoxazol-6-yl and 1,2-benzisoxazol-7-yl. The term “2,1-benzisoxazolyl” as used herein includes 2,1-benzisoxazol-3-yl, 2,1-benzisoxazol-4-yl, 2,1-benzisoxazol-5-yl, 2,1-benzisoxazol-6-yl and 2,1-benzisoxazol-7-yl. The term “1,3-benzothiazolyl” as used herein includes 1,3-benzothiazol-2-yl, 1,3-benzothiazol-4-yl, 1,3-benzothiazol-5-yl, 1,3-benzothiazol-6-yl and 1,3-benzothiazol-7-yl. The term “1,2-benzoisothiazolyl” as used herein includes 1,2-benzisothiazol-3-yl, 1,2-benzisothiazol-4-yl, 1,2-benzisothiazol-5-yl, 1,2-benzisothiazol-6-yl and 1,2-benzisothiazol-7-yl. The term “2,1-benzoisothiazolyl” as used herein includes 2,1-benzisothiazol-3-yl, 2,1-benzisothiazol-4-yl, 2,1-benzisothiazol-5-yl, 2,1-benzisothiazol-6-yl and 2,1-benzisothiazol-7-yl. The term “benzotriazolyl” as used herein includes benzotriazol-1-yl, benzotriazol-4-yl, benzotriazol-5-yl, benzotriazol-6-yl and benzotriazol-7-yl. The term “1,2,3-benzoxadiazolyl” as used herein includes 1,2,3-benzoxadiazol-4-yl, 1,2,3-benzoxadiazol-5-yl, 1,2,3-benzoxadiazol-6-yl and 1,2,3-benzoxadiazol-7-yl. The term “2,1,3-benzoxadiazolyl” as used herein includes 2,1,3-benzoxadiazol-4-yl, 2,1,3-benzoxadiazol-5-yl, 2,1,3-benzoxadiazol-6-yl and 2,1,3-benzoxadiazol-7-yl. The term “1,2,3-benzothiadiazolyl” as used herein includes 1,2,3-benzothiadiazol-4-yl, 1,2,3-benzothiadiazol-5-yl, 1,2,3-benzothiadiazol-6-yl and 1,2,3-benzothiadiazol-7-yl. The term “2,1,3-benzothiadiazolyl” as used herein includes 2,1,3 benzothiadiazol-4-yl, 2,1,3-benzothiadiazol-5-yl, 2,1,3-benzothiadiazol-6-yl and 2,1,3-benzothiadiazol-7-yl. The term “thienopyridinyl” as used herein includes thieno[2,3-b]pyridinyl, thieno[2,3-c]pyridinyl, thieno[3,2-c]pyridinyl and thieno[3,2-b]pyridinyl. The term “purinyl” as used herein includes purin-2-yl, purin-6-yl, purin-7-yl and purin-8-yl. The term “imidazo[1,2-a]pyridinyl”, as used herein includes imidazo[1,2-a]pyridin-2-yl, imidazo[1,2-a]pyridin-3-yl, imidazo[1,2-a]pyridin-4-yl, imidazo[1,2-a]pyridin-5-yl, imidazo[1,2-a]pyridin-6-yl and imidazo[1,2-a]pyridin-7-yl. The term “1,3-benzodioxolyl”, as used herein includes 1,3-benzodioxol-4-yl, 1,3-benzodioxol-5-yl, 1,3-benzodioxol-6-yl, and 1,3-benzodioxol-7-yl. The term “quinolinyl” as used herein includes quinolin-2-yl, quinolin-3-yl, quinolin-4-yl, quinolin-5-yl, quinolin-6-yl, quinolin-7-yl and quinolin-8-yl. The term “isoquinolinyl” as used herein includes isoquinolin-1-yl, isoquinolin-3-yl, isoquinolin-4-yl, isoquinolin-5-yl, isoquinolin-6-yl, isoquinolin-7-yl and isoquinolin-8-yl. The term “cinnolinyl” as used herein includes cinnolin-3-yl, cinnolin-4-yl, cinnolin-5-yl, cinnolin-6-yl, cinnolin-7-yl and cinnolin-8-yl. The term “quinazolinyl” as used herein includes quinazolin-2-yl, quinazolin-4-yl, quinazolin-5-yl, quinazolin-6-yl, quinazolin-7-yl and quinazolin-8-yl. The term “quinoxalinyl” as used herein includes quinoxalin-2-yl, quinoxalin-5-yl, and quinoxalin-6-yl.

The term “heteroaryloxy”, as a group or part of a group, refers to a group having the formula —O—R^{k} wherein R^{k} is heteroaryl as defined herein above.

The term “mono- or di-alkylamino”, as a group or part of a group, refers to a group of formula —N(R^{o})(R^{p}) wherein R^{o} and R^{p} are each independently selected from hydrogen, or alkyl, wherein at least one of R^{o} or R^{p} is alkyl. Thus, alkylamino include mono-alkyl amino group (e.g. mono-C₁₋₆alkylamino group such as methylamino and ethylamino), and di-alkylamino group (e.g. di-C₁₋₆alkylamino group such as dimethylamino and diethylamino). Non-limiting examples of suitable mono- or di-alkylamino groups include n-propylamino, isopropylamino, n-butylamino, i-butylamino, sec-butylamino, t-butylamino, pentylamino, n-hexylamino, di-n-propylamino, di-i-propylamino, ethylmethylamino, methyl-n-propylamino, methyl-1-propylamino, n-butylmethylamino, i-butylmethylamino, t-butylmethylamino, ethyl-n-propylamino, ethyl-1-propylamino, n-butylethylamino, i-butylethylamino, t-butylethylamino, di-n-butylamino, di-i-butylamino, methylpentylamino, methylhexylamino, ethylpentylamino, ethylhexylamino, propylpentylamino, propylhexylamino, and the like.

The term “mono- or di-arylamino”, as a group or part of a group, refers to a group of formula —N(R^{q})(R^{r}) wherein R^{q} and R^{r} are each independently selected from hydrogen, aryl, or alkyl, wherein at least one of R^{q} or R^{r} is aryl.

The term “mono- or di-cycloalkylamino”, as a group or part of a group, refers to a group of formula —N(R^{s})(R^{t}) wherein R^{s} and R^{t} are each independently selected from hydrogen, cycloalkyl, alkyl, wherein at least one of R^{s} or R^{t} is cycloalkyl.

The term “mono- or di-heteroarylamino”, as a group or part of a group, refers to a group of formula —N(R^{u})(R^{v}) wherein R^{u} and R^{v} are each independently selected from hydrogen, heteroaryl, or alkyl, wherein at least one of R^{u} or R^{v} is heteroaryl as defined herein.

The term “mono- or di-heterocyclylamino”, as a group or part of a group, refers to a group of formula —N(R^{w})(R^{x}) wherein R^{w} and R^{x} are each independently selected from hydrogen, heterocyclyl, or alkyl, wherein at least one of R^{w} or R^{x} is heterocyclyl as defined herein.

The term “alkyloxycarbonyl”, as a group or part of a group, refers to a group of formula —COO—R^{b}, wherein R^{b} is alkyl as defined herein.

The term “cycloalkyloxycarbonyl”, as a group or part of a group, refers to a group of formula —COO—R^{b}, wherein R^{b} is cycloalkyl as defined herein.

The term “aryloxycarbonyl”, as a group or part of a group, refers to a group of formula —COO—R^{b}, wherein R^{b} is aryl as defined herein.

The term “alkylsulfinyl”, as a group or part of a group, refers to a group of formula —SO—R^{b}, wherein R^{b} is alkyl as defined herein.

The term “alkylsulfonyl”, as a group or part of a group, refers to a group of formula —S(O)₂—R^{b}, wherein R^{b} is alkyl as defined herein.

The term “mono- or di-alkylaminosulfonyl”, as a group or part of a group, refers to a group of formula —S(O)₂—NNR^{o}R^{p}, wherein R^{o}R^{p} are each independently selected from hydrogen, or alkyl, wherein at least one of R^{o} or R^{p} is alkyl.

The term “mono- or dialkylaminocarbonyl”, as a group or part of a group, refers to a group of formula —CONR^{o}R^{p} wherein R^{o}R^{p} are each independently selected from hydrogen, or alkyl, wherein at least one of R^{o} or R^{p} is alkyl.

The term “mono- or dicycloalkylaminocarbonyl”, as a group or part of a group, refers to a group of formula —CONR^{o}R^{p} wherein R^{o}R^{p} are each independently selected from hydrogen, or cycloalkyl, wherein at least one of R^{o} or R^{p} is cycloalkyl.

The term “alkylcarbonyl”, as a group or part of a group, refers to a group of formula —CO—R^{b}, wherein R^{b} is alkyl as defined herein.

The term “cycloalkylcarbonyl”, as a group or part of a group, refers to a group of formula —CO—R^{b}, wherein R^{b} is cycloalkyl as defined herein.

The term “arylcarbonyl”, as a group or part of a group, refers to a group of formula —CO—R^{b}, wherein R^{b} is aryl as defined herein.

The term “alkylcarbonylamino”, as a group or part of a group, refers to a group of formula —NR^{o}—CO—R^{b}, wherein R^{o} is selected from hydrogen, or alkyl and R^{b} is alkyl as defined herein.

The term “alkylsulfonylamino”, as a group or part of a group, refers to a group of formula —NR^{o}—S(O)₂—R^{b}, wherein R^{o} is selected from hydrogen, or alkyl and R^{b} is alkyl as defined herein.

The term “a saturated or unsaturated 4-, 5-, 6-, 7-, 8- or 9-membered ring” as used herein encompasses saturated or unsaturated carbon only membered rings, as well as saturated or unsaturated heteroatoms containing rings. The term “a saturated 4-, 5-, 6-, 7-, 8- or 9-carbon membered ring” as used herein refers to saturated carbon only membered ring such as C₃₋₇cycloalkyl and C₃₋₇cycloalkylene

The term “a saturated or unsaturated 5-, 6-, 7-, 8-, 9-, 10- or 11-membered nitrogen-containing monocycle or bicycle” as used encompasses saturated or unsaturated nitrogen containing heterocycles comprising one or two cycles, for instance a azepanyl ring, an indolinyl ring, a benzimidazolyl ring. The term “an unsaturated 4-, 5-, 6-, 7-, 8- or 9-carbon membered ring” as used herein refers to unsaturated nitrogen containing heterocycles comprising one or two cycles such indolinyl, and benzimidazolyl.

Whenever used in the present invention the term “compounds of the invention” or a similar term is meant to include the compounds of general formula (I), (IA), (IB), (IC), (IIA), (IIB), (IIC), (IID) and any subgroup thereof. This term also refers to the compounds as depicted in Table 1 and their derivatives, N-oxides, salts, solvates, hydrates, tautomeric forms, analogues, pro-drugs, esters and metabolites, as well as their quaternized nitrogen analogues. The N-oxide forms of said compounds are meant to comprise compounds wherein one or several nitrogen atoms are oxidized to the so-called N-oxide.

As used herein and unless otherwise stated, the term “stereoisomer” refers to all possible different isomeric as well as conformational forms which the compounds of structural formula herein may possess, in particular all possible stereochemically and conformationally isomeric forms, all diastereomers, enantiomers and/or conformers of the basic molecular structure. Some compounds of the present invention may exist in different tautomeric forms, all of the latter being included within the scope of the present invention.

The present invention includes all possible stereoisomers compounds of formula (I) and any subgroup thereof. When a compound is desired as a single enantiomer, such may be obtained by stereospecific synthesis, by resolution of the final product or any convenient intermediate, or by chiral chromatographic methods as each are known in the art. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994), incorporated by reference with regard to stereochemistry. A structural isomer is a type of isomer in which molecules with the same molecular formula have different bonding patterns and atomic organization. Where structural isomers are interconvertible via a low energy barrier, tautomeric isomerism (‘tautomerism’) can occur. This can take the form of proton tautomerism in compounds of the invention containing, for example, an imino, keto, or oxime group, or so-called valence tautomerism in compounds which contain an aromatic moiety.

The term “prodrug” as used herein means the pharmacologically acceptable derivatives such as esters, amides and phosphates, such that the resulting in vivo biotransformation product of the derivative is the active drug. The reference by Goodman and Gilman (The Pharmacological Basis of Therapeutics, 8th Ed, McGraw-Hill, Int. Ed. 1992, “Biotransformation of Drugs”, p 13-15) describing pro-drugs generally is hereby incorporated. Prodrugs of the compounds of the invention can be prepared by modifying functional groups present in said component in such a way that the modifications are cleaved, either in routine manipulation or in vivo, to the parent component. Typical examples of prodrugs are described for instance in WO 99/33795, WO 99/33815, WO 99/33793 and WO 99/33792 all incorporated herein by reference. Prodrugs are characterized by increased bio-availability and are readily metabolized into the active inhibitors in vivo. The term “prodrug”, as used herein, means any compound that will be modified to form a drug species, wherein the modification may take place either inside or outside of the body, and either before or after the pre-drug reaches the area of the body where administration of the drug is indicated.

Preferred statements (features) and embodiments of the compounds and processes of this invention are now set forth. Each statement and embodiment of the invention so defined may be combined with any other statement and/or embodiments unless clearly indicated to the contrary. In particular, any feature indicated as being preferred or advantageous may be combined with any other feature or features indicated as being preferred or advantageous.

A first aspect of the present invention provides a compound of formula (I) or a stereoisomer, or tautomer thereof,

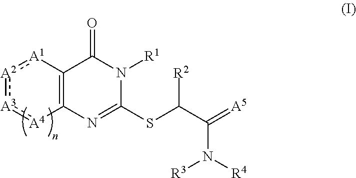

wherein,
each dotted line represents an optional double bond whereby maximally 2 non-adjacent dotted lines can form a double bond;
n is an integer selected from 0, 1, or 2;
A¹ is selected from the group consisting of CR⁵, S, N, NR⁶, and O;
A² is selected from the group consisting of CR⁵, S, N, NR⁶, and O;
A³ is selected from the group consisting of CR⁵, S, N, NR⁶, and O;
A⁴ is selected from the group consisting of CR⁵, S, N, NR⁶, and O;
wherein at most two of A¹ to A⁴ is selected from S, N, NR⁶, and O;
A⁵ is O or NR⁷;
R¹ is selected from the group consisting of alkyl, cycloalkyl, alkenyl, aryl, arylalkyl, heterocyclyl, and heteroaryl; wherein said alkyl, cycloalkyl, alkenyl, aryl, arylalkyl, heterocyclyl, or heteroaryl can be unsubstituted or substituted with one or more Z¹;
R² is selected from the group consisting of hydrogen, alkyl, cycloalkyl, aryl, arylalkyl, heterocyclyl, and heteroaryl; wherein said alkyl, cycloalkyl, aryl, arylalkyl, heterocyclyl, or heteroaryl can be unsubstituted or substituted with one or more Z²;
R³ is selected from the group consisting of hydrogen, cycloalkyl, aryl, alkyl, haloalkyl, haloalkyloxy, arylalkyl, heterocyclyl, heteroaryl, —C(O)R⁸, —CO₂R⁹, —C(O)NR⁹R¹⁰, —S(O)₂R⁹, and —SO₂NR⁹R¹⁰;
wherein said cycloalkyl, aryl, alkyl, arylalkyl, heterocyclyl, or heteroaryl can be unsubstituted or substituted with one or more Z³;
or R² and R³ together with the atom to which they are attached form a saturated or unsaturated 5-, 6-, 7-, 8- or 9-membered ring;
R⁴ is selected from the group consisting of hydrogen, cycloalkyl, aryl, alkyl, haloalkyl, haloalkyloxy, arylalkyl, heterocyclyl, and heteroaryl; wherein said cycloalkyl, aryl, alkyl, haloalkyl, haloalkyloxy, arylalkyl, heterocyclyl, or heteroaryl can be unsubstituted or substituted with one or more Z⁴;
or R³ and R⁴ together with the nitrogen atom to which they are attached form a saturated or unsaturated 5-, 6-, 7-, 8-, 9-, 10- or 11-membered nitrogen-containing monocycle or bicycle;
R⁵ is selected from the group consisting of hydrogen, alkyl, cycloalkyl, halo, aryl, arylalkyl, heterocyclyl, heteroaryl, hydroxyl, —OR⁸, cyano, amino, nitro, haloalkyl, haloalkyloxy, —C(O)R⁸, —NR⁸R⁹, —CO₂R⁹, —C(O)NR⁹R¹⁰, —S(O)₂R⁹, —S(O)₂NR⁹R¹⁰, —NR⁹C(O)R¹⁰, and —NR⁹S(O)₂R¹⁰;
or wherein two R⁵ together with the atom to which they are attached can form a saturated or unsaturated 5-, 6-, 7-, 8-, 9- or 10-membered ring;
wherein said ring, alkyl, cycloalkyl, aryl, arylalkyl, heterocyclyl, or heteroaryl can be unsubstituted or substituted with one or more Z⁵;
R⁶ is selected from the group consisting of hydrogen, alkyl, cycloalkyl, aryl, arylalkyl, heterocyclyl, heteroaryl, and —S(O)₂R⁹; wherein said alkyl, cycloalkyl, aryl, arylalkyl, heterocyclyl, or heteroaryl can be unsubstituted or substituted with one or more Z⁶;
R⁷ is selected from the group consisting of alkyl, cycloalkyl, aryl, arylalkyl, heterocyclyl, and heteroaryl; wherein said alkyl, cycloalkyl, aryl, arylalkyl, heterocyclyl, or heteroaryl can be unsubstituted or substituted with one or more Z⁷;
or R³ and R⁷ together with the atom to which they are attached form an unsaturated 5-, 6-, 7-, 8-, 9-, 10- or 11-membered nitrogen-containing monocycle or bicycle;
each R⁸ is independently selected from the group consisting of, alkyl, aryl, cycloalkyl, arylalkyl, heterocyclyl, heteroaryl;
each R⁹ is independently selected from the group consisting of hydrogen, alkyl, aryl, cycloalkyl, arylalkyl, heterocyclyl, heteroaryl;
each R¹⁰ is independently selected from the group consisting of hydrogen, alkyl, aryl, cycloalkyl, arylalkyl, heterocyclyl, heteroaryl;
each Z¹ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z² is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z³ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z⁴ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z⁵ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z⁶ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z⁷ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro; or a solvate, hydrate, pharmaceutically acceptable salt, or prodrug thereof. In particular embodiments, these compounds are envisaged for use in the the prevention or treatment of a disease associated with the activation of receptor tyrosine-protein kinase (ERBB4).

Another aspect of the present invention provides a compound of formula (I) or a stereoisomer, or tautomer thereof,

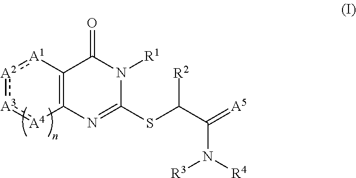

wherein,
each dotted line represents an optional double bond whereby maximally 2 non-adjacent dotted lines can form a double bond;
n is an integer selected from 0, 1, or 2;
A¹ is selected from the group consisting of CR⁵, S, N, NR⁶, and O;
A² is selected from the group consisting of CR⁵, S, N, NR⁶, and O;
A³ is selected from the group consisting of CR⁵, S, N, NR⁶, and O;
A⁴ is selected from the group consisting of CR⁵, S, N, NR⁶, and O;
wherein at most two of A¹ to A⁴ is selected from S, N, NR⁶, and O;
A⁵ is 0 or NR⁷;
R¹ is selected from the group consisting of alkyl, cycloalkyl, alkenyl, aryl, arylalkyl, heterocyclyl, and heteroaryl; wherein said alkyl, cycloalkyl, alkenyl, aryl, arylalkyl, heterocyclyl, or heteroaryl can be unsubstituted or substituted with one or more Z¹;
R² is selected from the group consisting of hydrogen, alkyl, cycloalkyl, aryl, arylalkyl, heterocyclyl, and heteroaryl; wherein said alkyl, cycloalkyl, aryl, arylalkyl, heterocyclyl, or heteroaryl can be unsubstituted or substituted with one or more Z²;
R³ is selected from the group consisting of hydrogen, cycloalkyl, aryl, alkyl, haloalkyl, haloalkyloxy, arylalkyl, heterocyclyl, heteroaryl, —C(O)R⁸, —CO₂R⁹, —C(O)NR⁹R¹⁰, —S(O)₂R⁹, and —SO₂NR⁹R¹⁰;
wherein said cycloalkyl, aryl, alkyl, arylalkyl, heterocyclyl, or heteroaryl can be unsubstituted or substituted with one or more Z³;
or R² and R³ together with the atom to which they are attached form a saturated or unsaturated 5-, 6-, 7-, 8- or 9-membered ring;
R⁴ is selected from the group consisting of hydrogen, cycloalkyl, aryl, alkyl, haloalkyl, haloalkyloxy, arylalkyl, heterocyclyl, and heteroaryl; wherein said cycloalkyl, aryl, alkyl, haloalkyl, haloalkyloxy, arylalkyl, heterocyclyl, or heteroaryl can be unsubstituted or substituted with one or more Z⁴;
or R³ and R⁴ together with the nitrogen atom to which they are attached form a saturated or unsaturated 5-, 6-, 7-, 8-, 9-, 10- or 11-membered nitrogen-containing monocycle or bicycle;
R⁵ is selected from the group consisting of hydrogen, alkyl, cycloalkyl, halo, aryl, arylalkyl, heterocyclyl, heteroaryl, hydroxyl, —OR^{B}, cyano, amino, nitro, haloalkyl, haloalkyloxy, —C(O)R⁸, —NR⁸R⁹, —CO₂R⁹, —C(O)NR⁹R¹⁰, —S(O)₂R⁹, —S(O)₂NR⁹R¹⁰, —NR⁹C(O)R¹⁰ and —NR⁹S(O)₂R¹⁰;
or wherein two R⁵ together with the atom to which they are attached can form a saturated or unsaturated 5-, 6-, 7-, 8-, 9- or 10-membered ring;
wherein said ring, alkyl, cycloalkyl, aryl, arylalkyl, heterocyclyl, or heteroaryl can be unsubstituted or substituted with one or more Z⁵;
R⁶ is selected from the group consisting of hydrogen, alkyl, cycloalkyl, aryl, arylalkyl, heterocyclyl, heteroaryl, and —S(O)₂R⁹; wherein said alkyl, cycloalkyl, aryl, arylalkyl, heterocyclyl, or heteroaryl can be unsubstituted or substituted with one or more Z⁶;
R⁷ is selected from the group consisting of alkyl, cycloalkyl, aryl, arylalkyl, heterocyclyl, and heteroaryl; wherein said alkyl, cycloalkyl, aryl, arylalkyl, heterocyclyl, or heteroaryl can be unsubstituted or substituted with one or more Z⁷;
or R³ and R⁷ together with the atom to which they are attached form an unsaturated 5-, 6-, 7-, 8-, 9-, 10- or 11-membered nitrogen-containing monocycle or bicycle;
each R⁸ is independently selected from the group consisting of, alkyl, aryl, cycloalkyl, arylalkyl, heterocyclyl, heteroaryl;
each R⁹ is independently selected from the group consisting of hydrogen, alkyl, aryl, cycloalkyl, arylalkyl, heterocyclyl, heteroaryl;
each R¹⁰ is independently selected from the group consisting of hydrogen, alkyl, aryl, cycloalkyl, arylalkyl, heterocyclyl, heteroaryl;
each Z¹ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z² is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z³ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z⁴ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z⁵ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z⁶ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z⁷ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro; or a solvate, hydrate, pharmaceutically acceptable salt, or prodrug thereof. In particular embodiments, the compounds are envisaged for use as a medicament.

According to particular embodiments, the present invention provides compounds of formula (I) wherein A¹ is CR⁵, A² is CR⁵ and A⁵ is O.

According to particular embodiments, the present invention provides compounds of formula (I) wherein A¹ is CR⁵, A² is CR⁵, A³ is CR⁵ or S, A⁴ is CR⁵ and A⁵ is O.

According to particular embodiments, the present invention provides compounds of formula (I) wherein A¹ is CR⁵, A² is CR⁵, A⁵ is O; and
wherein when n is 0 then A³ is S; or
wherein when n is 1 then A³ is CR⁵ and A⁴ is CR⁵.

In some embodiments the compound according to the present invention has structural formula (IAA),

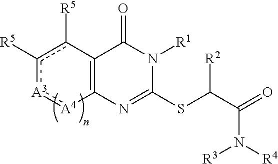

wherein n, A³, A⁴, R¹, R², R³, R⁴, and R⁵ have the same meaning as that defined herein; preferably wherein when n is 0 then A³ is S; or wherein when n is 1 then A³ is CR⁵ and A⁴ is CR⁵.

In some embodiments the compound according to the present invention has structural formula (IA), (IB) or (IC)

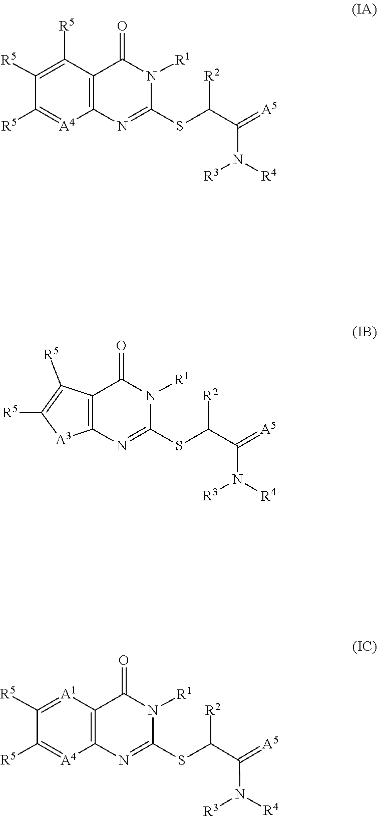

wherein A¹, A⁴, A⁵, R¹, R², R³, R⁴, and R⁵ have the same meaning as that defined herein.

In some embodiments the compound according to the present invention has structural formula (IIA), (IIB), (IIC) or (IID)

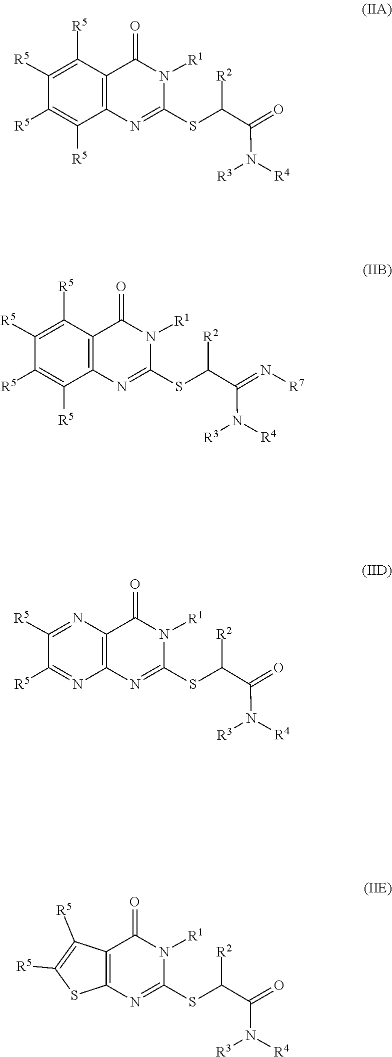

wherein R¹, R², R³, R⁴, and R⁵ have the same meaning as that defined herein. Preferably the compound according to the present invention have structural formula (IIA) or (IIE).

According to particular embodiments, the present invention provides compounds of formula (I), and any subgroup thereof such as (IAA), (IA), (IB), (IC), (IIA), (IIB), (IIC), (IID), wherein,
A¹ is CR⁵, N, or S;
A² is CR⁵, N,or S;
A³ is CR⁵, N,or S;
A⁴ is CR⁵, N,or S;
R¹ is selected from the group consisting of alkyl, cycloalkyl, alkenyl, aryl, and arylalkyl; wherein said alkyl, cycloalkyl, alkenyl, aryl, and arylalkyl can be unsubstituted or substituted with one or more Z¹;
R² is selected from the group consisting of hydrogen, alkyl, cycloalkyl, and aryl;
R³ is selected from the group consisting of hydrogen, cycloalkyl, aryl, alkyl, haloalkyl, haloalkyloxy, arylalkyl, heterocyclyl, heteroaryl, —OR⁸, —CO₂R⁹, —C(O)NR⁹R¹⁰, —S(O)₂R⁹, and —SO₂NR⁹R¹⁰;
wherein said cycloalkyl, aryl, alkyl, arylalkyl, heterocyclyl, or heteroaryl can be unsubstituted or substituted with one or more Z³;
or R² and R³ together with the atom to which they are attached form a saturated or unsaturated 5-, 6-, 7- 8- or 9-membered ring;
R⁴ is selected from the group consisting of hydrogen, cycloalkyl, aryl, alkyl, and haloalkyl;
or R³ and R⁴ together with the nitrogen atom to which they are attached form a saturated or unsaturated 7-, 8-, 9-, or 10-membered nitrogen-containing monocycle or bicycle;
R⁵ is selected from the group consisting of hydrogen, alkyl, cycloalkyl, halo, aryl, arylalkyl, heterocyclyl, heteroaryl, hydroxyl, —OR⁸, cyano, amino, nitro, haloalkyl, haloalkyloxy, —C(O)R⁸, —NR⁸R⁹, —CO₂R⁹, —C(O)NR⁹R¹⁰, —S(O)₂R⁹, —SO₂NR⁹R¹⁰, —NR⁹C(O)R¹⁰, and —NR⁹S(O)₂R¹⁰;
or wherein two R⁵ together with the atom to which they are attached can form a saturated or unsaturated 5-, 6-, or 7-membered ring;
wherein said ring, alkyl, cycloalkyl, aryl, arylalkyl, heterocyclyl, or heteroaryl can be unsubstituted or substituted with one or more Z⁵;
R⁶ is selected from the group consisting of hydrogen, alkyl, and cycloalkyl;
R⁷ is selected from the group consisting of alkyl, cycloalkyl, aryl, arylalkyl, heterocyclyl, and heteroaryl;
or R³ and R⁷ together with the atom to which they are attached form an unsaturated 6-, 7-, 8-, 9-, or 10-membered nitrogen-containing monocycle or bicycle.

According to particular embodiments, the present invention provides compounds of formula (I), and any subgroup thereof such as (IAA), (IA), (IB), (IC), (IIA), (IIB), (IIC), (IID), wherein,
A¹ is CR⁵, A² is CR⁵, A³ is CR⁵ or S, A⁴ is CR⁵ and A⁵ is O.

According to particular embodiments, the present invention provides compounds of formula (I), and any subgroup thereof such as (IAA), (IA), (IB), (IC), (IIA), (IIB), (IIC), (IID), wherein, each Z¹ is independently selected from the group consisting of halo, C₁₋₆alkyl, haloC₁₋₄alkyl, haloC₁₋₄alkyloxy, C₃₋₁₂cycloalkyl, C₃₋₁₂cycloalkenyl, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, mono-C₁₋₄alkylamino, mono-C₃₋₁₂cycloalkylamino, mono-C₆₋₁₂arylamino, hydroxycarbonyl, C₁₋₄alkyloxycarbonyl, C₃₋₁₂cycloalkyloxycarbonyl, C₆₋₁₂aryloxycarbonyl, aminocarbonyl, mono-C₁₋₄alkylaminocarbonyl, mono-C₃₋₁₂cycloalkylaminocarbonyl, C₁₋₄alkylcarbonyl, C₃₋₁₂cycloalkylcarbonyl, C₆₋₁₂arylcarbonyl, —S(O)H, C₁₋₄alkylsulfinyl, —S(O)₂H, C₁₋₄alkylsulfonyl, —SO₂N H₂, mono-C₁₋₄alkylaminosulfonyl, nitro;
each Z² is independently selected from the group consisting of halo, C₁₋₆alkyl, haloC₁₋₄alkyl, haloC₁₋₄alkyloxy, C₃₋₁₂cycloalkyl, C₃₋₁₂cycloalkenyl, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, mono-C₁₋₄alkylamino, mono-C₃₋₁₂cycloalkylamino, mono-C₆₋₁₂arylamino, hydroxycarbonyl, C₁₋₄alkyloxycarbonyl, C₃₋₁₂cycloalkyloxycarbonyl, C₆₋₁₂aryloxycarbonyl, aminocarbonyl, mono-C₁₋₄alkylaminocarbonyl, mono-C₃₁₂cycloalkylaminocarbonyl, C₁₋₄ alkylcarbonyl, C₃₋₁₂cycloalkylcarbonyl, C₆₋₁₂arylcarbonyl, —S(O)H, C₁₋₄alkylsulfinyl, —S(O)₂H, C₁₋₄alkylsulfonyl, —SO₂NH₂, mono-C₁₋₄alkylaminosulfonyl, nitro;
each Z³ is independently selected from the group consisting of halo, C₁₋₆alkyl, haloC₁₋₄alkyl, haloC₁₋₄alkyloxy, C₃₋₁₂cycloalkyl, C₃₋₁₂cycloalkenyl, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, mono-C₁₋₄alkylamino, mono-C₃₋₁₂cycloalkylamino, mono-C₆₋₁₂arylamino, hydroxycarbonyl, C₁₋₄alkyloxycarbonyl, C₃₋₁₂cycloalkyloxycarbonyl, C₆₋₁₂aryloxycarbonyl, aminocarbonyl, mono-C₁₋₄alkylaminocarbonyl, mono-C₃₋₁₂cycloalkylaminocarbonyl, C₁₋₄alkylcarbonyl, C₃₋₁₂cycloalkylcarbonyl, C₆₋₁₂arylcarbonyl, —S(O)H, C₁₋₄alkylsulfinyl, —S(O)₂H, C₁₋₄alkylsulfonyl, —SO₂NH₂, mono-C₁₋₄alkylaminosulfonyl, nitro;
each Z⁴ is independently selected from the group consisting of halo, C₁₋₆alkyl, haloC₁₋₄alkyl, haloC₁₋₄alkyloxy, C₃₋₁₂cycloalkyl, C₃₋₁₂cycloalkenyl, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, mono-C₁₋₄alkylamino, mono-C₃₋₁₂cycloalkylamino, mono-C₆₋₁₂arylamino, hydroxycarbonyl, C₁₋₄alkyloxycarbonyl, C₃₋₁₂cycloalkyloxycarbonyl, C₆₋₁₂aryloxycarbonyl, aminocarbonyl, mono-C₁₋₄alkylaminocarbonyl, mono-C₃₋₁₂cycloalkylaminocarbonyl, C₁₋₄alkylcarbonyl, C₃₋₁₂cycloalkylcarbonyl, C₆₋₁₂arylcarbonyl, —S(O)H, C₁₋₄alkylsulfinyl, —S(O)₂H, C₁₋₄alkylsulfonyl, —SO₂NH₂, mono-C₁₋₄alkylaminosulfonyl, nitro;
each Z⁵ is independently selected from the group consisting of halo, C₁₋₆alkyl, haloC₁₋₄alkyl, haloC₁₋₄alkyloxy, C₃₋₁₂cycloalkyl, C₃₋₁₂cycloalkenyl, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, mono-C₁₋₄alkylamino, mono-C₃₋₁₂cycloalkylamino, mono-C₆₋₁₂arylamino, hydroxycarbonyl, C₁₋₄alkyloxycarbonyl, C₃₋₁₂cycloalkyloxycarbonyl, C₆₋₁₂aryloxycarbonyl, aminocarbonyl, mono-C₁₋₄alkylaminocarbonyl, mono-C₃₋₁₂cycloalkylaminocarbonyl, C₁₋₄alkylcarbonyl, C₃₋₁₂cycloalkylcarbonyl, C₆₋₁₂arylcarbonyl, —S(O)H, C₁₋₄-alkylsulfinyl, —S(O)₂H, C₁₋₄alkylsulfonyl, —SO₂NH₂, mono-C₁₋₄alkylaminosulfonyl, nitro;
each Z⁶ is independently selected from the group consisting of halo, C₁₋₆alkyl, haloC₁₋₄alkyl, haloC₁₋₄-alkyloxy, C₃₋₁₂cycloalkyl, C₃₋₁₂cycloalkenyl, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, mono-C₁₋₄alkylamino, mono-C₃₋₁₂cycloalkylamino, mono-C₆₋₁₂arylamino, hydroxycarbonyl, C₁₋₄-alkyloxycarbonyl, C₃₋₁₂cycloalkyloxycarbonyl, C₆₋₁₂aryloxycarbonyl, aminocarbonyl, mono-C₁₋₄alkylaminocarbonyl, mono-C₃₋₁₂cycloalkylaminocarbonyl, C₁₋₄alkylcarbonyl, C₃₋₁₂cycloalkylcarbonyl, C₆₋₁₂arylcarbonyl, —S(O)H, C₁₋₄-alkylsulfinyl, —S(O)₂H, C₁₋₄alkylsulfonyl, —SO₂NH₂, mono-C₁₋₄alkylaminosulfonyl, nitro;
each Z⁷ is independently selected from the group consisting of halo, C₁₋₆alkyl, haloC₁₋₄alkyl, haloC₁₋₄alkyloxy, C₃₋₁₂cycloalkyl, C₃₋₁₂cycloalkenyl, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, mono-C₁₋₄alkylamino, mono-C₃₋₁₂cycloalkylamino, mono-C₆₋₁₂arylamino, hydroxycarbonyl, C₁₋₄alkyloxycarbonyl, C₃₋₁₂cycloalkyloxycarbonyl, C₆₋₁₂aryloxycarbonyl, aminocarbonyl, mono-C₁₋₄alkylaminocarbonyl, mono-C₃₋₁₂cycloalkylaminocarbonyl, C₁₋₄alkylcarbonyl, C₃₋₁₂cycloalkylcarbonyl, C₆₋₁₂arylcarbonyl, —S(O)H, C₁₋₄alkylsulfinyl, —S(O)₂H, C₁₋₄alkylsulfonyl, —SO₂NH₂, mono-C₁₋₄alkylaminosulfonyl, nitro. In particular embodiments, these compounds are envisaged for use a medicament. In further particular embodiments, the compounds are envisaged for use in the prevention or treatment of a disease associated with the activation of receptor tyrosine-protein kinase (ERBB4);

According to particular embodiments, the present invention provides compounds of formula (I), and any subgroup thereof such as (IAA), (IA), (IB), (IC), (IIA), (IIB), (IIC), (IID),
wherein,
R¹ is selected from the group consisting of C₁₋₆alkyl, C₃₋₁₂cycloalkyl, C₂₋₆alkenyl, C₆₋₁₂aryl, C₆₋₁₂arylC₁₋₆alkyl and heteroaryl; wherein said groups can be unsubstituted or substituted with one, two or three Z¹;
R² is selected from the group consisting of hydrogen, C₁₋₆alkyl, C₃₋₁₂cycloalkyl and C₆₋₁₂aryl;
R³ is selected from the group consisting of hydrogen, C₃₋₁₂cycloalkyl, C₆₋₁₂aryl, C₁₋₆alkyl, haloC₁₋₄alkyl, haloC₁₋₄alkyloxy, C₆₋₁₂arylC₁₋₆alkyl, heterocyclyl, heteroaryl, C₁₋₄alkylcarbonyl, C₃₋₁₂cycloalkylcarbonyl, C₆₋₁₂arylcarbonyl, C₁₋₄alkyloxycarbonyl, C₃₋₁₂cycloalkyloxycarbonyl, C₆₋₁₂aryloxycarbonyl, aminocarbonyl, mono-C₁₋₄alkylaminocarbonyl, and mono-C₃₋₁₂cycloalkylaminocarbonyl; wherein said groups can be unsubstituted or substituted with one, two or three Z³;
or R² and R³ together with the atom to which they are attached form a saturated unsaturated 6-, 7-, or 8-membered ring;
R⁴ is selected from the group consisting of hydrogen, C₃₋₁₀cycloalkyl, C₆₋₁₂aryl, and haloC₁₋₄alkyl;
or R³ and R⁴ together with the nitrogen atom to which they are attached form a saturated 7-, 8-, 9- or 10-membered nitrogen-containing monocycle or bicycle;
R⁵ is selected from the group consisting of hydrogen, C₁₋₆alkyl, C₃₋₁₂cycloalkyl, halo, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, nitro, haloC₁₋₄alkyl, haloC₁₋₄alkyloxy, C₁₋₄alkylcarbonyl, C₃₋₂cycloalkylcarbonyl, C₆₋₁₂arylcarbonyl, mono-C₁₋₄alkylamino, mono-C₃₋₁₂ cycloalkylamino, mono-C₆₋₁₂arylamino, hydroxycarbonyl, C₁₋₄alkyloxycarbonyl, C₃₋₁₂ cycloalkyloxycarbonyl, C₆₋₁₂aryloxycarbonyl, aminocarbonyl, mono-C₁₋₄alkylaminocarbonyl, mono-C₃₋₁₂cycloalkylaminocarbonyl, S(O)₂H, C₁₋₄akylsulfonyl, —SO₂N H₂, mono-C₁₋₄alkylaminosulfonyl, C₁₋₄alkylcarbonylamino, and C₁₋₄alkylsulfonylamino;
or wherein two R⁵ together with the atom to which they are attached can form a saturated or unsaturated 5-, 6-, or 7-membered ring;
wherein said ring, C₁₋₆alkyl, C₃₋₁₂cycloalkyl, C₆₋₁₂aryl, C₆₋₁₂arylC₁₋₆alkyl, heterocyclyl, or heteroaryl can be unsubstituted or substituted with one, two or three Z⁵;
R⁶ is selected from the group consisting of hydrogen, C₁₋₆alkyl and C₃₋₁₂cycloalkyl;
R⁷ is selected from the group consisting of C₁₋₆alkyl, C₃₋₁₂cycloalkyl, C₆₋₁₂aryl, C₆₋₁₂arylalkyl, heterocyclyl, and heteroaryl;
or R³ and R⁷ together with the atom to which they are attached form an unsaturated 7-, 8-, 9-, or 10-membered nitrogen-containing monocycle or bicycle. In particular embodiments, these compounds are envisaged for use as a medicament. In further embodiments the compounds are envisaged for use in the prevention or treatment of a disease associated with the activation of receptor tyrosine-protein kinase (ERBB4) as described herein.

According to particular embodiments, the present invention provides compounds of formula (I), and any subgroup thereof such as (IAA), (IA), (IB), (IC), (IIA), (IIB), (IIC), (IID), wherein,
A¹ is CR⁵, A² is CR⁵, A³ is CR⁵ or S, A⁴ is CR⁵ and A⁵ is O;
R¹ is selected from the group consisting of C₁₋₆alkyl, C₃₋₁₂cycloalkyl, C₂₋₆alkenyl, C₆₋₁₂aryl, C₆₋₁₂arylC₁₋₆alkyl and heteroaryl; preferably R¹ is C₁₋₆alkyl, C₃₋₁₂cycloalkyl, C₆₋₁₂aryl, and C₆₋₁₂arylC₁₋₆ alky; R¹ is C₁₋₆alkyl, C₃₋₁₀cycloalkyl, and C₆₋₁₂aryl;
wherein said groups can be unsubstituted or substituted with one, two or three Z¹;
R² is selected from the group consisting of hydrogen, C₁₋₆alkyl, C₃₋₁₂cycloalkyl and C₆₋₁₂aryl;
R³ is selected from the group consisting of hydrogen, C₃₋₁₂cycloalkyl, C₆₋₁₂aryl, C₁₋₆alkyl, haloC₁₋₄alkyl, haloC₁₋₄alkyloxy, C₆₋₁₂arylC₁₋₆alkyl, heterocyclyl, heteroaryl, C₁₋₄alkylcarbonyl, C₃₋₁₂cycloalkylcarbonyl, C₆₋₁₂arylcarbonyl, C₁₋₄alkyloxycarbonyl, C₃₋₁₂cycloalkyloxycarbonyl, C₆₋₁₂aryloxycarbonyl, aminocarbonyl, mono-C₁₋₄alkylaminocarbonyl, and mono-C₃₋₁₂cycloalkylaminocarbonyl; preferably R³ is hydrogen, C₃₋₁₂cycloalkyl, C₆₋₁₂aryl, C₁₋₆alkyl, haloC₁₋₄alkyl, haloC₁₋₄alkyloxy, heterocyclyl, heteroaryl, C₁₋₄alkylcarbonyl, C₃₋₁₂cycloalkylcarbonyl, C₆₋₁₂ arylcarbonyl, aminocarbonyl, mono-C₁₋₄alkylaminocarbonyl, and mono-C₃₋₁₂ cycloalkylaminocarbonyl wherein said groups can be unsubstituted or substituted with one, two or three Z³;
or R² and R³ together with the atom to which they are attached form a saturated unsaturated 6-, 7-, or 8-membered ring;
R⁴ is selected from the group consisting of hydrogen, C₃₋₁₀cycloalkyl, C₆₋₁₂aryl, and haloC₁₋₄alkyl;
or R³ and R⁴ together with the nitrogen atom to which they are attached form a saturated 7-, 8-, 9- or 10-membered nitrogen-containing monocycle or bicycle;
R⁵ is selected from the group consisting of hydrogen, C₁₋₆alkyl, C₃₋₁₂cycloalkyl, halo, C₆₋₁₂aryl, C₆₋₁₂ arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, nitro, haloC₁₋₄alkyl, haloC₁₋₄alkyloxy, C₁₋₄alkylcarbonyl, C₃₋₁₂cycloalkylcarbonyl, C₆₋₁₂arylcarbonyl, mono-C₁₋₄alkylamino, mono-C₃₋₁₂ cycloalkylamino, mono-C₆₋₁₂arylamino, hydroxycarbonyl, C₁₋₄alkyloxycarbonyl, C₃₋₁₂ cycloalkyloxycarbonyl, C₆₋₁₂aryloxycarbonyl, aminocarbonyl, mono-C₁₋₄alkylaminocarbonyl, mono-C₃₋₁₂cycloalkylaminocarbonyl, S(O)₂H, C₁₋₄alkylsulfonyl, —SO₂N H₂, mono-C₁₋₄alkylaminosulfonyl, C₁₋₄alkylcarbonylamino, and C₁₋₄alkylsulfonylamino; preferably R⁵ is hydrogen, C₁₋₆alkyl, C₃₋₁₂cycloalkyl, halo, C₆₋₁₂aryl, C₆₋₁₂arylC₁₋₆alkyl, heterocyclyl, heteroaryl, hydroxyl, C₁₋₄ alkyloxy, cyano, amino, nitro, haloC₁₋₄akyl, haloC₁₋₄alkyloxy, C₁₋₄alkylcarbonyl, C₃₋₁₂cycloalkylcarbonyl, mono-C₁₋₄alkylamino, mono-C₃₋₁₂cycloalkylamino, mono-C₆₋₁₂arylamino, hydroxycarbonyl, C₁₋₄alkyloxycarbonyl, C₆₋₁₂aryloxycarbonyl, aminocarbonyl, mono-C₁₋₄ alkylaminocarbonyl, C₁₋₄alkylsulfonyl, —SO₂NH₂, mono-C₁₋₄alkylaminosulfonyl, and C₁₋₄alkylsulfonylamino;
or wherein two R⁵ together with the atom to which they are attached can form a saturated or unsaturated 5-, 6-, or 7-membered ring;
wherein said ring, C₁₋₆alkyl, C₃₋₁₂cycloalkyl, C₆₋₁₂aryl, C₆₋₁₂arylC₁₋₆alkyl, heterocyclyl, or heteroaryl can be unsubstituted or substituted with one, two or three Z⁵;
R⁶ is selected from the group consisting of hydrogen, C₁₋₆alkyl and C₃₋₁₂cycloalkyl;
R⁷ is selected from the group consisting of C₁₋₆alkyl, C₃₋₁₂cycloalkyl, C₆₋₁₂aryl, C₆₋₁₂arylalkyl, heterocyclyl, and heteroaryl;
or R³ and R⁷ together with the atom to which they are attached form an unsaturated 7-, 8-, 9-, or 10-membered nitrogen-containing monocycle or bicycle. In particular embodiments, these compounds are envisaged for use as a medicament. In further embodiments the compounds are envisaged for use in the prevention or treatment of a disease associated with the activation of receptor tyrosine-protein kinase (ERBB4) as described herein.

According to particular embodiments, the present invention provides compounds of formula (I), and any subgroup thereof such as (IAA), (IA), (IB), (IC), (IIA), (IIB), (IIC), (IID), wherein,
R¹ is selected from the group consisting of C₁₋₄alkyl, C₃₋₁₀cycloalkyl, C₂₋₄alkenyl, C₆₋₁₂aryl, C₆₋₁₂ arylC₁₋₄alkyl and heteroaryl;
R² is selected from the group consisting of hydrogen, C₁₋₄alkyl, and C₆₋₁₂aryl;
R³ is selected from the group consisting of hydrogen, C₃₋₁₀cycloalkyl, C₆₋₁₂aryl, C₁₋₄alkyl, haloC₁₋₄alkyl, haloC₁₋₄alkyloxy, C₆₋₁₂arylC₁₋₄alkyl, heterocyclyl, heteroaryl, C₁₋₄alkylcarbonyl, C₁₋₄alkyloxycarbonyl, aminocarbonyl, mono-C₁₋₄alkylaminocarbonyl, and mono-C₃₋₁₂cycloalkylaminocarbonyl; wherein said groups can be unsubstituted or substituted with one, two or three Z³;
or R² and R³ together with the carbon atom to which they are attached form a saturated 6-, 7-, or 8-membered ring;
R⁴ is selected from the group consisting of hydrogen, C₃₋₈cycloalkyl, C₆₋₁₂aryl, and haloC₁₋₄alkyl;
or R³ and R⁴ together with the nitrogen atom to which they are attached form a saturated 7-, 8-, 9-membered nitrogen-containing monocycle or bicycle;
R⁵ is selected from the group consisting of hydrogen, C₁₋₄alkyl, C₃₋₁₀cycloalkyl, halo, C₆₋₁₂aryl, C₆₋₁₂arylC₁₋₄alkyl, heterocyclyl, heteroaryl, hydroxyl, C₁₋₆alkyloxy, C₃₋₁₀cycloalkyloxy, C₆₋₁₂aryloxy, cyano, amino, nitro, haloC₁₋₄alkyl, haloC₁₋₄alkyloxy, mono-C₁₋₄alkylamino, hydroxycarbonyl, C₁₋₄alkyloxycarbonyl, —CONH₂, mono-C₁₋₄alkylaminocarbonyl, S(O)₂H, C₁₋₄alkylsulfonyl, and —SO₂NH₂;
or wherein two R⁵ together with the atom to which they are attached can form a saturated or unsaturated 5-, 6-, or 7-membered ring;
wherein said ring, C₁₋₄alkyl, C₃₋₈cycloalkyl, C₆₋₁₂aryl, C₆₋₁₂aryC₁₋₄alkyl, heterocyclyl, or heteroaryl can be unsubstituted or substituted with one, two or three Z⁵;
R⁶ is selected from the group consisting of hydrogen, C₁₋₆alkyl and C₃₋₁₀cycloalkyl;
R⁷ is selected from the group consisting of C₁₋₆alkyl, C₃₋₁₀cycloalkyl, C₆₋₁₂aryl, C₆₋₁₂arylalkyl, heterocyclyl, and heteroaryl;
or R³ and R⁷ together with the carbon atom to which they are attached form an unsaturated 7-, 8-, 9-membered nitrogen-containing monocycle or bicycle.

According to particular embodiments, the present invention provides compounds of formula (I), and any subgroup thereof such as (IAA), (IA), (IB), (IC), (IIA), (IIB), (IIC), (IID), for use in the prevention or treatment of a disease associated with the activation of receptor tyrosine-protein kinase (ERBB4); or for use as a medicament wherein, n is an integer selected from 0, 1, or 2;
A¹ is selected from the group consisting of CR⁵, S, N, NR⁶, and O; preferably is selected from CR⁵, S, and N;
A² is selected from the group consisting of CR⁵, S, N, NR⁶, and O; preferably is selected from CR⁵, S, and N;
A³ is selected from the group consisting of CR⁵, S, N, NR⁶, and O; preferably is selected from CR⁵, S, and N;
A⁴ is selected from the group consisting of CR⁵, S, N, NR⁶, and O; preferably is selected from CR⁵, S, and N;
wherein at most two of A¹ to A⁴ is selected from S, N, NR⁶, and O;
A⁵ is 0 or NR⁷;
R¹ is selected from the group consisting of alkyl, cycloalkyl, alkenyl, aryl, arylalkyl, heterocyclyl, and heteroaryl; preferably R¹ is selected from alkyl, cycloalkyl, alkenyl, aryl, arylalkyl, and heteroaryl;
wherein said groups can be unsubstituted or substituted with one or more Z¹; preferably said groups can be unsubstituted or substituted with one, two or three Z¹;
R² is selected from the group consisting of hydrogen, alkyl, cycloalkyl, aryl, arylalkyl, heterocyclyl, and heteroaryl; preferably R² is selected from hydrogen, alkyl, aryl, heterocyclyl, and heteroaryl; preferably R² is selected from hydrogen, alkyl, aryl, and heteroaryl;
wherein said groups can be unsubstituted or substituted with one or more Z²; preferably said groups can be unsubstituted or substituted with one, two or three Z²;
R³ is selected from the group consisting of hydrogen, cycloalkyl, aryl, alkyl, haloalkyl, haloalkyloxy, arylalkyl, heterocyclyl, heteroaryl, —C(O)R⁸, —CO₂R⁹, —C(O)NR⁹R¹⁰, —S(O)₂R⁹, and —SO₂NR⁹R¹⁰; preferably R³ is selected from hydrogen, cycloalkyl, aryl, alkyl, haloalkyl, haloalkyloxy, arylalkyl, heterocyclyl, heteroaryl, —CO₂R⁹, —C(O)NR⁹R¹⁰, and —S(O)₂R⁹; preferably R³ is selected from hydrogen, cycloalkyl, aryl, alkyl, haloalkyl, haloalkyloxy, arylalkyl, heteroaryl, and —C(O)NR⁹R¹⁰;
wherein said groups can be unsubstituted or substituted with one or more Z³; preferably said groups can be unsubstituted or substituted with one, two or three Z³;
or R² and R³ together with the atom to which they are attached form a saturated or unsaturated 5-, 6-, 7-, 8- or 9-membered ring; preferably form a saturated or unsaturated 6-, 7-, or 8-membered ring; preferably form a saturated 6-, 7-, or 8-membered ring;
R⁴ is selected from the group consisting of hydrogen, cycloalkyl, aryl, alkyl, haloalkyl, haloalkyloxy, arylalkyl, heterocyclyl, and heteroaryl; preferably R⁴ is selected from hydrogen, cycloalkyl, aryl, alkyl, haloalkyl, heterocyclyl, and heteroaryl; preferably R⁴ is selected from hydrogen, cycloalkyl, aryl, alkyl, and haloakyl;
wherein said groups can be unsubstituted or substituted with one or more Z⁴; preferably said groups can be unsubstituted or substituted with one, two or three Z⁴;
or R³ and R⁴ together with the nitrogen atom to which they are attached form a saturated or unsaturated 5-, 6-, 7-, 8-, 9-, 10- or 11-membered nitrogen-containing monocycle or bicycle; preferably form a saturated or unsaturated 6-, 7-, 8-, 9-, or 10-membered nitrogen-containing monocycle or bicycle; preferably form a saturated or unsaturated 6-, 7-, 8-, 9-, or 10-membered nitrogen-containing bicycle;
R⁵ is selected from the group consisting of hydrogen, alkyl, cycloalkyl, halo, aryl, arylalkyl, heterocyclyl, heteroaryl, hydroxyl, —OR⁸, cyano, amino, nitro, haloalkyl, haloalkyloxy, —C(O)R⁸, —NR⁸R⁹, —CO₂R⁹, —C(O)NR⁹R¹⁰, —S(O)₂R⁹, —S(O)₂NR⁹R¹⁰, —NR⁹C(O)R¹⁰ and —NR⁹S(O)₂R¹⁰; preferably R⁵ is selected from hydrogen, alkyl, cycloalkyl, halo, aryl, heterocyclyl, heteroaryl, hydroxyl, —OR⁸, cyano, amino, haloalkyl, haloalkyloxy, —NR⁸R⁹, —CO₂R⁹, —C(O)NR⁹R¹⁰, —S(O)₂R⁹, —S(O)₂NR⁹R¹⁰; preferably R⁵ is selected from hydrogen, alkyl, cycloalkyl, halo, aryl, heterocyclyl, heteroaryl, hydroxyl, —OR⁸, cyano, amino, haloalkyl, —CO₂R⁹, —C(O)NR⁹R¹⁰;
or wherein two R⁵ together with the atom to which they are attached can form an unsaturated 5-, 6-, 7-, 8-, 9-, 10- or 11-membered ring; preferably form a saturated or unsaturated 5-, 6-, 7-, 8-, 9-membered ring; preferably form an unsaturated 5-, 6-, 7-, 8-, 9-membered ring;
wherein said ring, or groups can be unsubstituted or substituted with one or more Z⁵; preferably said ring or groups can be unsubstituted or substituted with one, two or three Z⁵;
R⁶ is selected from the group consisting of hydrogen, alkyl, cycloalkyl, aryl, arylalkyl, heterocyclyl, heteroaryl, and —S(O)₂R⁹; preferably R⁶ is selected from hydrogen, alkyl, cycloalkyl, aryl, heterocyclyl, and heteroaryl; preferably R⁶ is selected from hydrogen, alkyl, and aryl;
wherein said groups can be unsubstituted or substituted with one or more Z⁶; preferably said groups can be unsubstituted or substituted with one, two or three Z⁶;
R⁷ is selected from the group consisting of alkyl, cycloalkyl, aryl, arylalkyl, heterocyclyl, and heteroaryl; preferably R⁷ is selected from hydrogen, alkyl, cycloalkyl, aryl, heterocyclyl, and heteroaryl; preferably R⁷ is selected from hydrogen, alkyl, and aryl;
wherein said groups can be unsubstituted or substituted with one or more Z⁷; preferably said groups can be unsubstituted or substituted with one, two or three Z⁷;
or R³ and R⁷ together with the atom to which they are attached form an unsaturated 5-, 6-, 7-, 8-, 9-, 10- or 11-membered nitrogen-containing monocycle or bicycle; preferably form a saturated or unsaturated 6-, 7-, 8-, 9-, or 10-membered nitrogen-containing monocycle or bicycle; preferably form a saturated or unsaturated 6-, 7-, 8-, 9-, or 10-membered nitrogen-containing bicycle;
each R⁸ is independently selected from the group consisting of alkyl, aryl, cycloalkyl, arylalkyl, heterocyclyl, and heteroaryl; preferably R⁸ is selected from alkyl, aryl, cycloalkyl, heterocyclyl, and heteroaryl; preferably R⁸ is selected from alkyl, aryl, and cycloalkyl;
each R⁹ is independently selected from the group consisting of hydrogen, alkyl, aryl, cycloalkyl, arylalkyl, heterocyclyl, heteroaryl; preferably R⁹ is selected from hydrogen, alkyl, aryl, cycloalkyl, heterocyclyl, and heteroaryl; preferably R⁹ is selected from hydrogen, alkyl, aryl, and cycloalkyl;
each R¹⁰ is independently selected from the group consisting of hydrogen, alkyl, aryl, cycloalkyl, arylalkyl, heterocyclyl, heteroaryl; preferably R¹⁰ is selected from hydrogen, alkyl, aryl, cycloalkyl, heterocyclyl, and heteroaryl; preferably R¹⁰ is selected from hydrogen, alkyl, aryl, and cycloalkyl;
each Z¹ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro; preferably Z¹ is selected from halo, alkyl, haloalkyl, haloalkyloxy, cycloalkyl, cycloalkenyl, aryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)₂R⁹, —S(O)₂NR⁹R¹⁰; preferably Z¹ is selected from halo, alkyl, cycloalkyl, cycloalkenyl, aryl, heterocyclyl, heteroaryl, hydroxyl, —OR⁸, cyano, amino, —NR⁸R⁹, —C(O)₂R⁹, —C(O)NR⁹R¹⁰, and —C(O)R⁸; preferably Z¹ is selected from halo, alkyl, cycloalkyl, cycloalkenyl, aryl, heterocyclyl, heteroaryl, hydroxyl, —OR⁸, cyano, —C(O)NR⁹R¹⁰ and —C(O)R⁸;
each Z² is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro; preferably Z² is selected from halo, alkyl, haloalkyl, haloalkyloxy, cycloalkyl, cycloalkenyl, aryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)₂R⁹, —S(O)₂NR⁹R¹⁰; preferably Z² is selected from halo, alkyl, cycloalkyl, cycloalkenyl, aryl, heterocyclyl, heteroaryl, hydroxyl, —OR⁸, cyano, amino, —NR⁸R⁹, —C(O)₂R⁹, —C(O)NR⁹R¹⁰, and —C(O)R⁸; preferably Z² is selected from halo, alkyl, cycloalkyl, cycloalkenyl, aryl, heterocyclyl, heteroaryl, hydroxyl, —OR⁸, cyano, —C(O)NR⁹R¹⁰ and —C(O)R⁸;
each Z³ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro; preferably Z³ is selected from halo, alkyl, haloalkyl, haloalkyloxy, cycloalkyl, cycloalkenyl, aryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)₂R⁹, —S(O)₂NR⁹R¹⁰; preferably Z³ is selected from halo, alkyl, cycloalkyl, cycloalkenyl, aryl, heterocyclyl, heteroaryl, hydroxyl, —OR⁸, cyano, amino, —NR⁸R⁹, —C(O)₂R⁹, —C(O)NR⁹R¹⁰, and —C(O)R⁸; preferably Z³ is selected from halo, alkyl, cycloalkyl, cycloalkenyl, aryl, heterocyclyl, heteroaryl, hydroxyl, —OR⁸, cyano, —C(O)NR⁹R¹⁰ and —C(O)R⁸;
each Z⁴ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro; preferably Z⁴ is selected from halo, alkyl, haloalkyl, haloalkyloxy, cycloalkyl, cycloalkenyl, aryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)₂R⁹, —S(O)₂NR⁹R¹⁰; preferably Z⁴ is selected from halo, alkyl, cycloalkyl, cycloalkenyl, aryl, heterocyclyl, heteroaryl, hydroxyl, —OR⁸, cyano, amino, —NR⁸R⁹, —C(O)₂R⁹, —C(O)NR⁹R¹⁰, and —C(O)R⁸; preferably Z⁴ is selected from halo, alkyl, cycloalkyl, cycloalkenyl, aryl, heterocyclyl, heteroaryl, hydroxyl, —OR⁸, cyano, —C(O)NR⁹R¹⁰ and —C(O)R⁸;
each Z⁵ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro; preferably Z⁵ is selected from halo, alkyl, haloalkyl, haloalkyloxy, cycloalkyl, cycloalkenyl, aryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)₂R⁹, —S(O)₂NR⁹R¹⁰; preferably Z⁵ is selected from halo, alkyl, cycloalkyl, cycloalkenyl, aryl, heterocyclyl, heteroaryl, hydroxyl, —OR⁸, cyano, amino, —NR⁸R⁹, —C(O)₂R⁹, —C(O)NR⁹R¹⁰, and —C(O)R⁸; preferably Z⁵ is selected from halo, alkyl, cycloalkyl, cycloalkenyl, aryl, heterocyclyl, heteroaryl, hydroxyl, —OR⁸, cyano, —C(O)NR⁹R¹⁰ and —C(O)R⁸;
each Z⁶ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro; preferably Z⁶ is selected from halo, alkyl, haloalkyl, haloalkyloxy, cycloalkyl, cycloalkenyl, aryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)₂R⁹, —S(O)₂NR⁹R¹⁰; preferably Z⁶ is selected from halo, alkyl, cycloalkyl, cycloalkenyl, aryl, heterocyclyl, heteroaryl, hydroxyl, —OR⁸, cyano, amino, —NR⁸R⁹, —C(O)₂R⁹, —C(O)NR⁹R¹⁰, and —C(O)R⁸; preferably Z⁶ is selected from halo, alkyl, cycloalkyl, cycloalkenyl, aryl, heterocyclyl, heteroaryl, hydroxyl, —OR⁸, cyano, —C(O)NR⁹R¹⁰ and —C(O)R⁸;
each Z⁷ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro; preferably Z⁷ is selected from halo, alkyl, haloalkyl, haloalkyloxy, cycloalkyl, cycloalkenyl, aryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)₂R⁹, —S(O)₂NR⁹R¹⁰; preferably Z⁷ is selected from halo, alkyl, cycloalkyl, cycloalkenyl, aryl, heterocyclyl, heteroaryl, hydroxyl, —OR⁸, cyano, amino, —NR⁸R⁹, —C(O)₂R⁹, —C(O)NR⁹R¹⁰, and —C(O)R⁸; preferably Z⁷ is selected from halo, alkyl, cycloalkyl, cycloalkenyl, aryl, heterocyclyl, heteroaryl, hydroxyl, —OR⁸, cyano, —C(O)NR⁹R¹⁰ and —C(O)R⁸.

In particular embodiments, these compounds are envisaged for use as a medicament. In further particular embodiments, the compounds are envisaged for use in the the prevention or treatment of a disease associated with the activation of receptor tyrosine-protein kinase (ERBB4) as described herein.

According to particular embodiments, the present invention provides compounds of formula (I), and any subgroup thereof such as (IAA), (IA), (IB), (IC), (IIA), (IIB), (IIC), (IID), wherein,
R¹ is selected from the group consisting of C₁₋₆alkyl, C₃₋₁₂cycloalkyl, C₂₋₆alkenyl, C₆₋₁₂aryl, C₆₋₁₂ arylC₁₋₆alkyl, heterocyclyl, and heteroaryl; preferably R¹ is selected from C₁₋₆alkyl, C₃₋₁₂ cycloalkyl, C₂₋₆alkenyl, C₆₋₁₂aryl, C₆₋₁₂arylC₁₋₆alkyl, and heteroaryl;
wherein said groups can be unsubstituted or substituted with one or more Z¹; preferably said groups can be unsubstituted or substituted with one, two or three Z¹;
R² is selected from the group consisting of hydrogen, C₁₋₆alkyl, C₃₋₁₂cycloalkyl, C₆₋₁₂aryl, C₆₋₁₂ arylC₁₋₆alkyl, heterocyclyl, and heteroaryl; preferably R² is selected from hydrogen, C₁₋₆alkyl, C₆₋₁₂ aryl, heterocyclyl, and heteroaryl; preferably R² is selected from hydrogen, C₁₋₆alkyl, C₆₋₁₂aryl, and heteroaryl;
wherein said groups can be unsubstituted or substituted with one or more Z²; preferably said groups can be unsubstituted or substituted with one, two or three Z²;
R³ is selected from the group consisting of hydrogen, C₃₋₁₂cycloalkyl, C₆₋₁₂aryl, C₁₋₆alkyl, haloC₁₋₄alkyl, haloC₁₋₄alkyloxy, C₆₋₁₂arylC₁₋₆alkyl, heterocyclyl, heteroaryl, —C(O)R⁸, —CO₂R⁹, —C(O)NR⁹R¹⁰, —S(O)₂R⁹, and —SO₂NR⁹R¹⁰; preferably R³ is selected from hydrogen, C₃₋₁₂cycloalkyl, C₆₋₁₂aryl, C₁₋₆alkyl, haloC₁₋₄alkyl, haloC₁₋₄alkyloxy, C₆₋₁₂arylC₁₋₆alkyl, heterocyclyl, heteroaryl, —CO₂R⁹, —C(O)NR⁹R¹⁰, and —S(O)₂R⁹; preferably R³ is selected from hydrogen, C₃₋₁₂cycloalkyl, C₆₋₁₂aryl, C₁₋₆alkyl, haloC₁₋₄alkyl, haloC₁₋₄alkyloxy, C₆₋₁₂arylC₁₋₆alkyl, heteroaryl, and —C(O)NR⁹R¹⁰;
wherein said groups can be unsubstituted or substituted with one or more Z³; preferably said groups can be unsubstituted or substituted with one, two or three Z³;
or R² and R³ together with the atom to which they are attached form a saturated or unsaturated 5-, 6-, 7-, 8- or 9-membered ring; preferably form a saturated or unsaturated 6-, 7-, or 8-membered ring; preferably form a saturated 6-, 7-, or 8-membered ring;
R⁴ is selected from the group consisting of hydrogen, C₃₋₁₂cycloalkyl, C₆₋₁₂aryl, C₁₋₆alkyl, haloC₁₋₄alkyl, haloC₁₋₄alkyloxy, C₆₋₁₂arylC₁₋₆alkyl, heterocyclyl, and heteroaryl; preferably R⁴ is selected from hydrogen, C₃₋₁₂cycloalkyl, C₆₋₁₂aryl, C₁₋₆alkyl, haloC₁₋₄alkyl, heterocyclyl, and heteroaryl; preferably R⁴ is selected from hydrogen, C₃₋₁₂cycloalkyl, C₆₋₁₂aryl, C₁₋₆alkyl, and haloC₁₋₄alkyl;
wherein said groups can be unsubstituted or substituted with one or more Z⁴; preferably said groups can be unsubstituted or substituted with one, two or three Z⁴;
or R³ and R⁴ together with the nitrogen atom to which they are attached form a saturated or unsaturated 5-, 6-, 7-, 8-, 9-, 10- or 11-membered nitrogen-containing monocycle or bicycle; preferably form a saturated or unsaturated 6-, 7-, 8-, 9-, or 10-membered nitrogen-containing monocycle or bicycle; preferably form a saturated or unsaturated 6-, 7-, 8-, 9-, or 10-membered nitrogen-containing bicycle;
R⁵ is selected from the group consisting of hydrogen, C₁₋₆alkyl, C₃₋₁₂cycloalkyl, halo, C₆₋₁₂aryl, C₆₋₁₂arylC₁₋₆alkyl, heterocyclyl, heteroaryl, hydroxyl, —OR⁸, cyano, amino, nitro, haloC₁₋₄alkyl, haloC₁₋₄alkyloxy, —C(O)R⁸, —NR⁸R⁹, —CO₂R⁹, —C(O)NR⁹R¹⁰, —S(O)₂R⁹, —S(O)₂NR⁹R¹⁰, —NR⁹C(O)R¹⁰, and —NR⁹S(O)₂R¹⁰; preferably R⁵ is selected from hydrogen, C₁₋₆alkyl, C₃₋₁₂cycloalkyl, halo, C₆₋₁₂aryl, heterocyclyl, heteroaryl, hydroxyl, —OR^{B}, cyano, amino, haloC₁₋₄alkyl, haloC₁₋₄ alkyloxy, —NR⁸R⁹, —CO₂R⁹, —C(O)NR⁹R¹⁰, —S(O)₂R⁹, —S(O)₂NR⁹R¹⁰; preferably R⁵ is selected from hydrogen, C₁₋₆alkyl, C₃₋₁₂cycloalkyl, halo, C₆₋₁₂aryl, heterocyclyl, heteroaryl, hydroxyl, —OR⁸, cyano, amino, haloC₁₋₄alkyl, —CO₂R⁹, —C(O)NR⁹R¹⁰;
or wherein two R⁵ together with the atom to which they are attached can form an unsaturated 5-, 6-, 7-, 8-, 9-, 10- or 11-membered ring; preferably form a saturated or unsaturated 5-, 6-, 7-, 8-, 9-membered ring; preferably form an unsaturated 5-, 6-, 7-, 8-, 9-membered ring;
wherein said ring, or groups can be unsubstituted or substituted with one or more Z⁵; preferably said ring or groups can be unsubstituted or substituted with one, two or three Z⁵;
R⁶ is selected from the group consisting of hydrogen, C₁₋₆alkyl, C₃₋₁₂cycloalkyl, C₆₋₁₂aryl, C₆₋₁₂arylC₁₋₆alkyl, heterocyclyl, heteroaryl, and —S(O)₂R⁹; preferably R⁶ is selected from hydrogen, C₁₋₆alkyl, C₃₋₁₂cycloalkyl, aryl, heterocyclyl, and heteroaryl; preferably R⁶ is selected from hydrogen, C₁₋₆alkyl, and C₆₋₁₂aryl;
wherein said groups can be unsubstituted or substituted with one or more Z⁶; preferably said groups can be unsubstituted or substituted with one, two or three Z⁶;
R⁷ is selected from the group consisting of C₁₋₆alkyl, C₃₋₁₂cycloalkyl, C₆₋₁₂aryl, C₆₋₁₂arylC₁₋₆alkyl, heterocyclyl, and heteroaryl; preferably R⁷ is selected from hydrogen, C₁₋₆alkyl, C₃₋₁₂cycloalkyl, C₆₋₁₂aryl, heterocyclyl, and heteroaryl; preferably R⁷ is selected from hydrogen, C₁₋₆alkyl, and C₆₋₁₂aryl;
wherein said groups can be unsubstituted or substituted with one or more Z⁷; preferably said groups can be unsubstituted or substituted with one, two or three Z⁷;
or R³ and R⁷ together with the atom to which they are attached form an unsaturated 5-, 6-, 7-, 8-, 9-, 10- or 11-membered nitrogen-containing monocycle or bicycle; preferably form a saturated or unsaturated 6-, 7-, 8-, 9-, or 10-membered nitrogen-containing monocycle or bicycle; preferably form a saturated or unsaturated 6-, 7-, 8-, 9-, or 10-membered nitrogen-containing bicycle;
each R⁸ is independently selected from the group consisting of C₁₋₆alkyl, C₆₋₁₂aryl, C₃₋₁₂cycloalkyl, C₆₋₁₂arylC₁₋₆alkyl, heterocyclyl, and heteroaryl; preferably R⁸ is selected from C₁₋₆alkyl, C₆₋₁₂aryl, C₃₋₁₂cycloalkyl, heterocyclyl, and heteroaryl; preferably R⁸ is selected from C₁₋₆alkyl, C₆₋₁₂aryl, and C₃₋₁₂cycloalkyl;
each R⁹ is independently selected from the group consisting of hydrogen, C₁₋₆alkyl, C₆₋₁₂aryl, C₃₋₁₂cycloalkyl, C₆₋₁₂arylC₁₋₆alkyl, heterocyclyl, heteroaryl; preferably R⁹ is selected from hydrogen, C₁₋₆alkyl, C₆₋₁₂aryl, C₃₋₁₂cycloalkyl, heterocyclyl, and heteroaryl; preferably R⁹ is selected from hydrogen, C₁₋₆alkyl, C₆₋₁₂aryl, and C₃₋₁₂cycloalkyl;
each R¹⁰ is independently selected from the group consisting of hydrogen, C₁₋₆alkyl, C₆₋₁₂aryl, C₃₋₁₂ cycloalkyl, C₆₋₁₂arylC₁₋₆alkyl, heterocyclyl, heteroaryl; preferably R¹⁰ is selected from hydrogen, C₁₋₆alkyl, C₆₋₁₂aryl, C₃₋₁₂cycloalkyl, heterocyclyl, and heteroaryl; preferably R¹⁰ is selected from hydrogen, C₁₋₆alkyl, C₆₋₁₂aryl, and C₃₋₁₂cycloalkyl;
each Z¹ is independently selected from the group consisting of halo, C₁₋₆alkyl, haloC₁₋₄alkyl, haloC₁₋₄alkyloxy, C₃₋₁₂cycloalkyl, C₃₋₁₂cycloalkenyl, C₆₋₁₂aryl, C₆₋₁₂arylC₁₋₆alkyl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro; preferably Z¹ is selected from halo, C₁₋₆alkyl, haloC₁₋₄alkyl, haloC₁₋₄alkyloxy, C₃₋₁₂cycloalkyl, C₃₋₁₂ cycloalkenyl, C₆₋₁₂aryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)₂R⁹, —S(O)₂NR⁹R¹⁰; preferably Z¹ is selected from halo, C₁₋₆alkyl, C₃₋₁₂cycloalkyl, C₃₋₁₂cycloalkenyl, C₆₋₁₂aryl, heterocyclyl, heteroaryl, hydroxyl, —OR⁸, cyano, amino, —NR⁸R⁹, —C(O)₂R⁹, —C(O)NR⁹R¹⁰, and —C(O)R⁸; preferably Z¹ is selected from halo, C₁₋₆alkyl, C₃₋₁₂cycloalkyl, C₃₋₁₂cycloalkenyl, C₆₋₁₂aryl, heterocyclyl, heteroaryl, hydroxyl, —OR⁸, cyano, —C(O)NR⁹R¹⁰, and —C(O)R⁸;
each Z² is independently selected from the group consisting of halo, C₁₋₆alkyl, haloC₁₋₄alkyl, haloC₁₋₄alkyloxy, C₃₋₁₂cycloalkyl, C₃₋₁₂cycloalkenyl, C₆₋₁₂aryl, C₆₋₁₂arylC₁₋₆alkyl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro; preferably Z² is selected from halo, C₁₋₆alkyl, haloC₁₋₄alkyl, haloC₁₋₄alkyloxy, C₃₋₁₂cycloalkyl, C₃₋₁₂cycloalkenyl, C₆₋₁₂aryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)₂R⁹, —S(O)₂NR⁹R¹⁰; preferably Z² is selected from halo, C₁₋₆alkyl, C₃₋₁₂cycloalkyl, C₃₋₁₂cycloalkenyl, C₆₋₁₂aryl, heterocyclyl, heteroaryl, hydroxyl, —OR⁸, cyano, amino, —NR⁸R⁹, —C(O)₂R⁹, —C(O)NR⁹R¹⁰, and —C(O)R⁸; preferably Z² is selected from halo, C₁₋₆alkyl, C₃₋₁₂cycloalkyl, C₃₋₁₂cycloalkenyl, C₆₋₁₂aryl, heterocyclyl, heteroaryl, hydroxyl, —OR⁸, cyano, —C(O)NR⁹R¹⁰, and —C(O)R⁸;
each Z³ is independently selected from the group consisting of halo, C₁₋₆alkyl, haloC₁₋₄alkyl, haloC₁₋₄alkyloxy, C₃₋₁₂cycloalkyl, C₃₋₁₂cycloalkenyl, C₆₋₁₂aryl, C₆₋₁₂arylC₁₋₆alkyl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro; preferably Z³ is selected from halo, C₁₋₆alkyl, haloC₁₋₄alkyl, haloC₁₋₄alkyloxy, C₃₋₁₂cycloalkyl, C₃₋₁₂ cycloalkenyl, C₆₋₁₂aryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)₂R⁹, —S(O)₂NR⁹R¹⁰; preferably Z³ is selected from halo, C₁₋₆alkyl, C₃₋₁₂cycloalkyl, C₃₋₁₂cycloalkenyl, C₆₋₁₂aryl, heterocyclyl, heteroaryl, hydroxyl, —OR⁸, cyano, amino, —NR⁸R⁹, —C(O)₂R⁹, —C(O)NR⁹R¹⁰, and —C(O)R⁸; preferably Z³ is selected from halo, C₁₋₆alkyl, C₃₋₁₂cycloalkyl, C₃₋₁₂cycloalkenyl, C₆₋₁₂aryl, heterocyclyl, heteroaryl, hydroxyl, —OR⁸, cyano, —C(O)NR⁹R¹⁰, and —C(O)R⁸;
each Z⁴ is independently selected from the group consisting of halo, C₁₋₆alkyl, haloC₁₋₄alkyl, haloC₁₋alkyloxy, C₃₋₁₂cycloalkyl, C₃₋₁₂cycloalkenyl, C₆₋₁₂aryl, C₆₋₁₂arylC₁₋₆alkyl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro; preferably Z⁴ is selected from halo, C₁₋₆alkyl, haloC₁₋alkyl, haloC₁₋₄alkyloxy, C₃₋₁₂cycloalkyl, C₃₋₁₂ cycloalkenyl, C₆₋₁₂aryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)₂R⁹, —S(O)₂NR⁹R¹⁰; preferably Z⁴ is selected from halo, C₁₋₆alkyl, C₃₋₁₂cycloalkyl, C₃₋₁₂cycloalkenyl, C₆₋₁₂aryl, heterocyclyl, heteroaryl, hydroxyl, —OR⁸, cyano, amino, —NR⁸R⁹, —C(O)₂R⁹, —C(O)NR⁹R¹⁰, and —C(O)R⁸; preferably Z⁴ is selected from halo, C₁₋₆alkyl, C₃₋₁₂cycloalkyl, C₃₋₁₂cycloalkenyl, C₆₋₁₂aryl, heterocyclyl, heteroaryl, hydroxyl, —OR⁸, cyano, —C(O)NR⁹R¹⁰, and —C(O)R⁸;
each Z⁵ is independently selected from the group consisting of halo, C₁₋₆alkyl, haloC₁₋₄alkyl, haloC₁₋₄alkyloxy, C₃₋₁₂cycloalkyl, C₃₋₁₂cycloalkenyl, C₆₋₁₂aryl, C₆₋₁₂arylC₁₋₆alkyl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro; preferably Z⁵ is selected from halo, C₁₋₆alkyl, haloC₁₋₄alkyl, haloC₁₋alkyloxy, C₃₋₁₂cycloalkyl, C₃₋₁₂cycloalkenyl, C₆₋₁₂aryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)₂R⁹, —S(O)₂NR⁹R¹⁰; preferably Z⁵ is selected from halo, C₁₋₆alkyl, C₃₋₁₂cycloalkyl, C₃₋₁₂cycloalkenyl, C₆₋₁₂aryl, heterocyclyl, heteroaryl, hydroxyl, —OR⁸, cyano, amino, —NR⁸R⁹, —C(O)₂R⁹, —C(O)NR⁹R¹⁰, and —C(O)R⁸; preferably Z⁵ is selected from halo, C₁₋₆alkyl, C₃₋₁₂cycloalkyl, C₃₋₁₂cycloalkenyl, C₆₋₁₂aryl, heterocyclyl, heteroaryl, hydroxyl, —OR⁸, cyano, —C(O)NR⁹R¹⁰, and —C(O)R⁸;
each Z⁶ is independently selected from the group consisting of halo, C₁₋₆alkyl, haloC₁₋₄alkyl, haloC₁₋₄alkyloxy, C₃₋₁₂cycloalkyl, C₃₋₁₂cycloalkenyl, C₆₋₁₂aryl, C₆₋₁₂arylC₁₋₆alkyl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro; preferably Z⁶ is selected from halo, C₁₋₆alkyl, haloC₁₋₄alkyl, haloC₁₋₄alkyloxy, C₃₋₁₂cycloalkyl, C₃₋₁₂ cycloalkenyl, C₆₋₁₂aryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)₂R⁹, —S(O)₂NR⁹R¹⁰; preferably Z⁶ is selected from halo, C₁₋₆alkyl, C₃₋₁₂cycloalkyl, C₃₋₁₂cycloalkenyl, C₆₋₁₂aryl, heterocyclyl, heteroaryl, hydroxyl, —OR⁸, cyano, amino, —NR⁸R⁹, —C(O)₂R⁹, —C(O)NR⁹R¹⁰, and —C(O)R⁸; preferably Z⁶ is selected from halo, C₁₋₆alkyl, C₃₋₁₂cycloalkyl, C₃₋₁₂cycloalkenyl, C₆₋₁₂aryl, heterocyclyl, heteroaryl, hydroxyl, —OR⁸, cyano, —C(O)NR⁹R¹⁰, and —C(O)R⁸;
each Z⁷ is independently selected from the group consisting of halo, C₁₋₆alkyl, haloC₁₋₄alkyl, haloC₁₋alkyloxy, C₃₋₁₂cycloalkyl, C₃₋₁₂cycloalkenyl, C₆₋₁₂aryl, C₆₋₁₂arylC₁₋₆alkyl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro; preferably Z⁷ is selected from halo, C₁₋₆alkyl, haloC₁₋₄alkyl, haloC₁₋₄alkyloxy, C₃₋₁₂cycloalkyl, C₃₋₁₂ cycloalkenyl, C₆₋₁₂aryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)₂R⁹, —S(O)₂NR⁹R¹⁰; preferably Z⁷ is selected from halo, C₁₋₆alkyl, C₃₋₁₂cycloalkyl, C₃₋₁₂cycloalkenyl, C₆₋₁₂aryl, heterocyclyl, heteroaryl, hydroxyl, —OR⁸, cyano, amino, —NR⁸R⁹, —C(O)₂R⁹, —C(O)NR⁹R¹⁰, and —C(O)R⁸; preferably Z⁷ is selected from halo, C₁₋₆alkyl, C₃₋₁₂cycloalkyl, C₃₋₁₂cycloalkenyl, C₆₋₁₂aryl, heterocyclyl, heteroaryl, hydroxyl, —OR⁸, cyano, —C(O)NR⁹R¹⁰, and —C(O)R⁸.

In some embodiments R³ is selected from the group consisting of hydrogen, cycloalkyl, aryl, alkyl, haloalkyl, haloalkyloxy, arylalkyl, heterocyclyl, heteroaryl, —C(O)R⁸, —CO₂R⁹, —C(O)NR⁹R¹⁰, S(O)₂R⁹, and —SO₂NR⁹R¹⁰; preferably R³ is selected from hydrogen, cycloalkyl, aryl, alkyl, haloalkyl, haloalkyloxy, arylalkyl, heterocyclyl, heteroaryl, alkylcarbonyl, cycloalkylcarbonyl, arylcarbonyl, hydroxycarbonyl, alkyloxycarbonyl, cycloalkyloxycarbonyl, aryloxycarbonyl, aminocarbonyl, mono-alkylaminocarbonyl, di-alkylaminocarbonyl, mono-cycloalkylaminocarbonyl, —S(O)₂H, alkylsulfonyl, —SO₂N H₂, mono-alkylaminosulfonyl; preferably R³ is selected from hydrogen, cycloalkyl, aryl, alkyl, haloalkyl, haloalkyloxy, arylalkyl, heterocyclyl, heteroaryl, alkylcarbonyl, cycloalkylcarbonyl, hydroxycarbonyl, alkyloxycarbonyl, cycloalkyloxycarbonyl, aminocarbonyl, mono-alkylaminocarbonyl, mono-cycloalkylaminocarbonyl, —S(O)₂H, —SO₂NH₂; preferably R³ is selected from hydrogen, cycloalkyl, aryl, alkyl, haloalkyl, haloalkyloxy, arylalkyl, heterocyclyl, heteroaryl, hydroxycarbonyl, aminocarbonyl, mono-alkylaminocarbonyl, mono-cycloalkylaminocarbonyl; preferably R³ is selected from hydrogen, cycloalkyl, aryl, alkyl, arylalkyl, heteroaryl, hydroxycarbonyl, aminocarbonyl, mono-alkylaminocarbonyl, mono-cycloalkylaminocarbonyl; wherein said groups can be unsubstituted or substituted with one or more Z³; preferably said groups can be unsubstituted or substituted with one, two or three Z³.

In some embodiments R³ is selected from the group consisting of hydrogen, C₃₋₁₂cycloalkyl, C₆₋₁₂ aryl, C₁₋₆alkyl, haloC₁₋₄alkyl, haloC₁₋₄alkyloxy, C₆₋₁₂arylC₁₋₆alkyl, heterocyclyl, heteroaryl, C(O)R⁸, —CO₂R⁹, —C(O)NR⁹R¹⁰, —S(O)₂R⁹, and —SO₂NR⁹R¹⁰; preferably R³ is selected from hydrogen, C₃₋₁₂cycloalkyl, C₆₋₁₂aryl, C₁₋₆alkyl, haloC₁₋₄alkyl, haloC₁₋₄alkyloxy, C₆₋₁₂arylC₁₋₆alkyl, heterocyclyl, heteroaryl, C₁₋₆alkyl carbonyl, C₃₋₈cycloalkylcarbonyl, C₆₋₁₂arylarylcarbonyl, hydroxycarbonyl, C₁₋₆alkyl oxycarbonyl, C₃₋₈cycloalkyloxycarbonyl, C₆₋₁₂arylaryloxycarbonyl, aminocarbonyl, mono-C₁₋₆alkyl aminocarbonyl, di-C₁₋₆alkyl aminocarbonyl, mono-C₃₋₈cycloalkylaminocarbonyl, —S(O)₂H, C₁₋₆alkylsulfonyl, —SO₂NH₂, mono-C₁₋₆alkylaminosulfonyl; preferably R³ is selected from hydrogen, C₃₋₁₂cycloalkyl, C₆₋₁₂aryl, C₁₋₆alkyl, haloC₁₋₄alkyl, haloC₁₋₄alkyloxy, C₆₋₁₂arylC₁₋₆alkyl, heterocyclyl, heteroaryl, C₁₋₆alkyl carbonyl, C₃₋₈cycloalkylcarbonyl, hydroxycarbonyl, C₁₋₆alkyl oxycarbonyl, C₃₋₈cycloalkyloxycarbonyl, aminocarbonyl, mono-C₃₋₆alkylaminocarbonyl, mono-C₃₋₈cycloalkylaminocarbonyl, —S(O)₂H, —SO₂N H₂; preferably R³ is selected from hydrogen, C₃₋₁₀cycloalkyl, C₆₋₁₂aryl, C₁₋₆alkyl, haloC₁₋alkyl, haloC₁₋₄alkyloxy, C₆₋₁₂arylC₁₋₆alkyl, heterocyclyl, heteroaryl, hydroxycarbonyl, aminocarbonyl, mono-C₁₋₆alkylaminocarbonyl, mono-C₃₋₈cycloalkylaminocarbonyl; preferably R³ is selected from hydrogen, C₃₋₈cycloalkyl, C₆₋₁₂aryl, C₁₋₆alkyl, C₆₋₁₂arylC₁₋₄alkyl, heteroaryl, hydroxycarbonyl, aminocarbonyl, mono-C₁₋₆alkyl aminocarbonyl, mono-C₃₋₈cycloalkylaminocarbonyl; wherein said groups can be unsubstituted or substituted with one or more Z³; preferably said groups can be unsubstituted or substituted with one, two or three Z³.

In some embodiments R⁵ is selected from the group consisting of hydrogen, alkyl, cycloalkyl, halo, aryl, arylalkyl, heterocyclyl, heteroaryl, hydroxyl, —OW, cyano, amino, nitro, haloalkyl, haloalkyloxy, —C(O)R⁸, —NR⁸R⁹, —CO₂R⁹, —C(O)NR⁹R¹⁰, —S(O)₂R⁹, —S(O)₂NR⁹R¹⁰, —NR⁹C(O)R¹⁰ and —NR⁹S(O)₂R¹⁰; preferably R⁵ is selected from hydrogen, alkyl, cycloalkyl, halo, aryl, arylalkyl, heterocyclyl, heteroaryl, hydroxyl, alkyloxy, cycloalkyloxy, aryloxy, heterocyclyloxy, heteroaryloxy, cyano, amino, nitro, haloalkyl, haloalkyloxy, alkylcarbonyl, cycloalkylcarbonyl, arylcarbonyl, mono-alkylamino, di-alkylamino, mono-cycloalkylamino, di-cycloalkylamino, mono-arylamino, di-arylamino, mono-heterocyclylamino, di-heterocyclylamino, mono-heteroarylamino, di-heteroarylamino, hydroxycarbonyl, alkyloxycarbonyl, cycloalkyloxycarbonyl, aryloxycarbonyl, aminocarbonyl, mono-alkylaminocarbonyl, di-alkylaminocarbonyl, mono-cycloalkylaminocarbonyl, —S(O)₂H, alkylsulfonyl, —S(O)₂N H₂, mono-alkylaminosulfonyl, di-alkylaminosulfonyl, alkylcarbonylamino, and alkylsulfonylamino; preferably R⁵ is selected from hydrogen, alkyl, cycloalkyl, halo, aryl, arylalkyl, heterocyclyl, heteroaryl, hydroxyl, alkyloxy, cycloalkyloxy, aryloxy, cyano, amino, haloalkyl, haloalkyloxy, alkylcarbonyl, cycloalkylcarbonyl, arylcarbonyl, mono-alkylamino, mono-cycloalkylamino, mono-arylamino, mono-heterocyclylamino, mono-heteroarylamino, hydroxycarbonyl, alkyloxycarbonyl, cycloalkyloxycarbonyl, aminocarbonyl, mono-alkylaminocarbonyl, mono-cycloalkylaminocarbonyl, —S(O)₂H, alkylsulfonyl, —S(O)₂N H₂, and mono-alkylaminosulfonyl; preferably R⁵ is selected from hydrogen, alkyl, cycloalkyl, halo, aryl, arylalkyl, heterocyclyl, heteroaryl, hydroxyl, alkyloxy, cyano, amino, haloalkyl, haloalkyloxy, alkylcarbonyl, mono-alkylamino, hydroxycarbonyl, alkyloxycarbonyl, aminocarbonyl, mono-alkylaminocarbonyl, mono-cycloalkylaminocarbonyl, —S(O)₂H and —S(O)₂NH₂; preferably R⁵ is selected from hydrogen, alkyl, cycloalkyl, halo, aryl, heterocyclyl, heteroaryl, hydroxyl, alkyloxy, haloalkyl, mono-alkylamino, hydroxycarbonyl, alkyloxycarbonyl, and aminocarbonyl; wherein said groups can be unsubstituted or substituted with one or more Z⁵; preferably said groups can be unsubstituted or substituted with one, two or three Z⁵.

In some embodiments each Z¹ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro; preferably Z¹ is selected from halo, alkyl, haloalkyl, haloalkyloxy, cycloalkyl, cycloalkenyl, aryl, alkylaryl, heterocyclyl, heteroaryl, hydroxyl, alkyloxy, cycloalkyloxy, aryloxy, heterocyclyloxy, heteroaryloxy, cyano, amino, mono-alkylamino, di-alkylamino, mono-cycloalkylamino, di-cycloalkylamino, mono-arylamino, di-arylamino, mono-heterocyclylamino, di-heterocyclylamino, mono-heteroarylamino, di-heteroarylamino, hydroxycarbonyl, alkyloxycarbonyl, cycloalkyloxycarbonyl, aryloxycarbonyl, aminocarbonyl, mono-alkylaminocarbonyl, di-alkylaminocarbonyl, mono-cycloalkylaminocarbonyl, alkylcarbonyl, cycloalkylcarbonyl, arylcarbonyl, —S(O)H, C₁₋₄-alkylsulfinyl, —S(O)₂H, C₁₋₄-alkylsulfonyl, —S(O)₂NH₂, mono-alkylaminosulfonyl, di-alkylaminosulfonyl, alkylcarbonylamino, alkylsulfonylamino, nitro; preferably Z¹ is selected from halo, alkyl, haloalkyl, haloalkyloxy, cycloalkyl, cycloalkenyl, aryl, alkylaryl, heterocyclyl, heteroaryl, hydroxyl, alkyloxy, cycloalkyloxy, aryloxy, cyano, amino, mono-alkylamino, mono-cycloalkylamino, mono-arylamino, hydroxycarbonyl, alkyloxycarbonyl, cycloalkyloxycarbonyl, aryloxycarbonyl, aminocarbonyl, mono-alkylaminocarbonyl, mono-cycloalkylaminocarbonyl, alkylcarbonyl, cycloalkylcarbonyl, —S(O)H, C₁₋₄-alkylsulfinyl, —S(O)₂H, C₁₋₄alkylsulfonyl, —S(O)₂NH₂, mono-alkylaminosulfonyl, alkylcarbonylamino, alkylsulfonylamino; preferably Z¹ is selected from halo, alkyl, haloalkyl, haloalkyloxy, cycloalkyl, cycloalkenyl, aryl, heterocyclyl, heteroaryl, hydroxyl, alkyloxy, cycloalkyloxy, cyano, amino, mono-alkylamino, hydroxycarbonyl, alkyloxycarbonyl, aminocarbonyl, mono-alkylaminocarbonyl, mono-cycloalkylaminocarbonyl, alkylcarbonyl; preferably Z¹ is selected from halo, alkyl, haloalkyl, cycloalkyl, cycloalkenyl, aryl, heterocyclyl, heteroaryl, hydroxyl, alkyloxy, cyano, amino, mono-alkylamino, aminocarbonyl, mono-cycloalkylaminocarbonyl, alkylcarbonyl.

In some embodiments each Z² is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro; preferably Z² is selected from halo, alkyl, haloalkyl, haloalkyloxy, cycloalkyl, cycloalkenyl, aryl, alkylaryl, heterocyclyl, heteroaryl, hydroxyl, alkyloxy, cycloalkyloxy, aryloxy, heterocyclyloxy, heteroaryloxy, cyano, amino, mono-alkylamino, di-alkylamino, mono-cycloalkylamino, di-cycloalkylamino, mono-arylamino, di-arylamino, mono-heterocyclylamino, di-heterocyclylamino, mono-heteroarylamino, di-heteroarylamino, hydroxycarbonyl, alkyloxycarbonyl, cycloalkyloxycarbonyl, aryloxycarbonyl, aminocarbonyl, mono-alkylaminocarbonyl, di-alkylaminocarbonyl, mono-cycloalkylaminocarbonyl, alkylcarbonyl, cycloalkylcarbonyl, arylcarbonyl, —S(O)H, C₁₋₄-alkylsulfinyl, —S(O)₂H, C₁₋₄-alkylsulfonyl, —S(O)₂NH₂, mono-alkylaminosulfonyl, di-alkylaminosulfonyl, alkylcarbonylamino, alkylsulfonylamino, nitro; preferably Z² is selected from halo, alkyl, haloalkyl, haloalkyloxy, cycloalkyl, cycloalkenyl, aryl, alkylaryl, heterocyclyl, heteroaryl, hydroxyl, alkyloxy, cycloalkyloxy, aryloxy, cyano, amino, mono-alkylamino, mono-cycloalkylamino, mono-arylamino, hydroxycarbonyl, alkyloxycarbonyl, cycloalkyloxycarbonyl, aryloxycarbonyl, aminocarbonyl, mono-alkylaminocarbonyl, mono-cycloalkylaminocarbonyl, alkylcarbonyl, cycloalkylcarbonyl, —S(O)H, C₁₋₄-alkylsulfinyl, —S(O)₂H, C₁₋₄alkylsulfonyl, —S(O)₂NH₂, mono-alkylaminosulfonyl, alkylcarbonylamino, alkylsulfonylamino; preferably Z² is selected from halo, alkyl, haloalkyl, haloalkyloxy, cycloalkyl, cycloalkenyl, aryl, heterocyclyl, heteroaryl, hydroxyl, alkyloxy, cycloalkyloxy, cyano, amino, mono-alkylamino, hydroxycarbonyl, alkyloxycarbonyl, aminocarbonyl, mono-alkylaminocarbonyl, mono-cycloalkylaminocarbonyl, alkylcarbonyl; preferably Z² is selected from halo, alkyl, haloalkyl, cycloalkyl, cycloalkenyl, aryl, heterocyclyl, heteroaryl, hydroxyl, alkyloxy, cyano, amino, mono-alkylamino, aminocarbonyl, mono-cycloalkylaminocarbonyl, alkylcarbonyl.

In some embodiments each Z³ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro; preferably Z³ is selected from halo, alkyl, haloalkyl, haloalkyloxy, cycloalkyl, cycloalkenyl, aryl, alkylaryl, heterocyclyl, heteroaryl, hydroxyl, alkyloxy, cycloalkyloxy, aryloxy, heterocyclyloxy, heteroaryloxy, cyano, amino, mono-alkylamino, di-alkylamino, mono-cycloalkylamino, di-cycloalkylamino, mono-arylamino, di-arylamino, mono-heterocyclylamino, di-heterocyclylamino, mono-heteroarylamino, di-heteroarylamino, hydroxycarbonyl, alkyloxycarbonyl, cycloalkyloxycarbonyl, aryloxycarbonyl, aminocarbonyl, mono-alkylaminocarbonyl, di-alkylaminocarbonyl, mono-cycloalkylaminocarbonyl, alkylcarbonyl, cycloalkylcarbonyl, arylcarbonyl, —S(O)H, C₁₋₄-alkylsulfinyl, —S(O)₂H, C₁₋₄-alkylsulfonyl, —S(O)₂NH₂, mono-alkylaminosulfonyl, di-alkylaminosulfonyl, alkylcarbonylamino, alkylsulfonylamino, nitro; preferably Z³ is selected from halo, alkyl, haloalkyl, haloalkyloxy, cycloalkyl, cycloalkenyl, aryl, alkylaryl, heterocyclyl, heteroaryl, hydroxyl, alkyloxy, cycloalkyloxy, aryloxy, cyano, amino, mono-alkylamino, mono-cycloalkylamino, mono-arylamino, hydroxycarbonyl, alkyloxycarbonyl, cycloalkyloxycarbonyl, aryloxycarbonyl, aminocarbonyl, mono-alkylaminocarbonyl, mono-cycloalkylaminocarbonyl, alkylcarbonyl, cycloalkylcarbonyl, —S(O)H, C₁₋₄alkylsulfinyl, —S(O)₂H, C₁₋₄alkylsulfonyl, —S(O)₂NH₂, mono-alkylaminosulfonyl, alkylcarbonylamino, alkylsulfonylamino; preferably Z³ is selected from halo, alkyl, haloalkyl, haloalkyloxy, cycloalkyl, cycloalkenyl, aryl, heterocyclyl, heteroaryl, hydroxyl, alkyloxy, cycloalkyloxy, cyano, amino, mono-alkylamino, hydroxycarbonyl, alkyloxycarbonyl, aminocarbonyl, mono-alkylaminocarbonyl, mono-cycloalkylaminocarbonyl, alkylcarbonyl; preferably Z³ is selected from halo, alkyl, haloalkyl, cycloalkyl, cycloalkenyl, aryl, heterocyclyl, heteroaryl, hydroxyl, alkyloxy, cyano, amino, mono-alkylamino, aminocarbonyl, mono-cycloalkylaminocarbonyl, alkylcarbonyl.

In some embodiments each Z⁴ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro; preferably Z⁴ is selected from halo, alkyl, haloalkyl, haloalkyloxy, cycloalkyl, cycloalkenyl, aryl, alkylaryl, heterocyclyl, heteroaryl, hydroxyl, alkyloxy, cycloalkyloxy, aryloxy, heterocyclyloxy, heteroaryloxy, cyano, amino, mono-alkylamino, di-alkylamino, mono-cycloalkylamino, di-cycloalkylamino, mono-arylamino, di-arylamino, mono-heterocyclylamino, di-heterocyclylamino, mono-heteroarylamino, di-heteroarylamino, hydroxycarbonyl, alkyloxycarbonyl, cycloalkyloxycarbonyl, aryloxycarbonyl, aminocarbonyl, mono-alkylaminocarbonyl, di-alkylaminocarbonyl, mono-cycloalkylaminocarbonyl, alkylcarbonyl, cycloalkylcarbonyl, arylcarbonyl, —S(O)H, C₁₋₄alkylsulfinyl, —S(O)₂H, C₁₋₄alkylsulfonyl, —S(O)₂NH₂, mono-alkylaminosulfonyl, di-alkylaminosulfonyl, alkylcarbonylamino, alkylsulfonylamino, nitro; preferably Z⁴ is selected from halo, alkyl, haloalkyl, haloalkyloxy, cycloalkyl, cycloalkenyl, aryl, alkylaryl, heterocyclyl, heteroaryl, hydroxyl, alkyloxy, cycloalkyloxy, aryloxy, cyano, amino, mono-alkylamino, mono-cycloalkylamino, mono-arylamino, hydroxycarbonyl, alkyloxycarbonyl, cycloalkyloxycarbonyl, aryloxycarbonyl, aminocarbonyl, mono-alkylaminocarbonyl, mono-cycloalkylaminocarbonyl, alkylcarbonyl, cycloalkylcarbonyl, —S(O)H, C₁₋₄alkylsulfinyl, —S(O)₂H, C₁₋₄alkylsulfonyl, —S(O)₂NH₂, mono-alkylaminosulfonyl, alkylcarbonylamino, alkylsulfonylamino; preferably Z⁴ is selected from halo, alkyl, haloalkyl, haloalkyloxy, cycloalkyl, cycloalkenyl, aryl, heterocyclyl, heteroaryl, hydroxyl, alkyloxy, cycloalkyloxy, cyano, amino, mono-alkylamino, hydroxycarbonyl, alkyloxycarbonyl, aminocarbonyl, mono-alkylaminocarbonyl, mono-cycloalkylaminocarbonyl, alkylcarbonyl; preferably Z⁴ is selected from halo, alkyl, haloalkyl, cycloalkyl, cycloalkenyl, aryl, heterocyclyl, heteroaryl, hydroxyl, alkyloxy, cyano, amino, mono-alkylamino, aminocarbonyl, mono-cycloalkylaminocarbonyl, alkylcarbonyl.

In some embodiments each Z⁵ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro; preferably Z⁵ is selected from halo, alkyl, haloalkyl, haloalkyloxy, cycloalkyl, cycloalkenyl, aryl, alkylaryl, heterocyclyl, heteroaryl, hydroxyl, alkyloxy, cycloalkyloxy, aryloxy, heterocyclyloxy, heteroaryloxy, cyano, amino, mono-alkylamino, di-alkylamino, mono-cycloalkylamino, di-cycloalkylamino, mono-arylamino, di-arylamino, mono-heterocyclylamino, di-heterocyclylamino, mono-heteroarylamino, di-heteroarylamino, hydroxycarbonyl, alkyloxycarbonyl, cycloalkyloxycarbonyl, aryloxycarbonyl, aminocarbonyl, mono-alkylaminocarbonyl, di-alkylaminocarbonyl, mono-cycloalkylaminocarbonyl, alkylcarbonyl, cycloalkylcarbonyl, arylcarbonyl, —S(O)H, C₁₋₄-alkylsulfinyl, —S(O)₂H, C₁₋₄-alkylsulfonyl, —S(O)₂NH₂, mono-alkylaminosulfonyl, di-alkylaminosulfonyl, alkylcarbonylamino, alkylsulfonylamino, nitro; preferably Z⁵ is selected from halo, alkyl, haloalkyl, haloalkyloxy, cycloalkyl, cycloalkenyl, aryl, alkylaryl, heterocyclyl, heteroaryl, hydroxyl, alkyloxy, cycloalkyloxy, aryloxy, cyano, amino, mono-alkylamino, mono-cycloalkylamino, mono-arylamino, hydroxycarbonyl, alkyloxycarbonyl, cycloalkyloxycarbonyl, aryloxycarbonyl, aminocarbonyl, mono-alkylaminocarbonyl, mono-cycloalkylaminocarbonyl, alkylcarbonyl, cycloalkylcarbonyl, —S(O)H, C₁₋₄-alkylsulfinyl, —S(O)₂H, C₁₋₄alkylsulfonyl, —S(O)₂NH₂, mono-alkylaminosulfonyl, alkylcarbonylamino, alkylsulfonylamino; preferably Z⁵ is selected from halo, alkyl, haloalkyl, haloalkyloxy, cycloalkyl, cycloalkenyl, aryl, heterocyclyl, heteroaryl, hydroxyl, alkyloxy, cycloalkyloxy, cyano, amino, mono-alkylamino, hydroxycarbonyl, alkyloxycarbonyl, aminocarbonyl, mono-alkylaminocarbonyl, mono-cycloalkylaminocarbonyl, alkylcarbonyl; preferably Z⁵ is selected from halo, alkyl, haloalkyl, cycloalkyl, cycloalkenyl, aryl, heterocyclyl, heteroaryl, hydroxyl, alkyloxy, cyano, amino, mono-alkylamino, aminocarbonyl, mono-cycloalkylaminocarbonyl, alkylcarbonyl.

In some embodiments each Z⁶ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro; preferably Z⁶ is selected from halo, alkyl, haloalkyl, haloalkyloxy, cycloalkyl, cycloalkenyl, aryl, alkylaryl, heterocyclyl, heteroaryl, hydroxyl, alkyloxy, cycloalkyloxy, aryloxy, heterocyclyloxy, heteroaryloxy, cyano, amino, mono-alkylamino, di-alkylamino, mono-cycloalkylamino, di-cycloalkylamino, mono-arylamino, di-arylamino, mono-heterocyclylamino, di-heterocyclylamino, mono-heteroarylamino, di-heteroarylamino, hydroxycarbonyl, alkyloxycarbonyl, cycloalkyloxycarbonyl, aryloxycarbonyl, aminocarbonyl, mono-alkylaminocarbonyl, di-alkylaminocarbonyl, mono-cycloalkylaminocarbonyl, alkylcarbonyl, cycloalkylcarbonyl, arylcarbonyl, —S(O)H, C₁₋₄-alkylsulfinyl, —S(O)₂H, C₁₋₄-alkylsulfonyl, —S(O)₂NH₂, mono-alkylaminosulfonyl, di-alkylaminosulfonyl, alkylcarbonylamino, alkylsulfonylamino, nitro; preferably Z⁶ is selected from halo, alkyl, haloalkyl, haloalkyloxy, cycloalkyl, cycloalkenyl, aryl, alkylaryl, heterocyclyl, heteroaryl, hydroxyl, alkyloxy, cycloalkyloxy, aryloxy, cyano, amino, mono-alkylamino, mono-cycloalkylamino, mono-arylamino, hydroxycarbonyl, alkyloxycarbonyl, cycloalkyloxycarbonyl, aryloxycarbonyl, aminocarbonyl, mono-alkylaminocarbonyl, mono-cycloalkylaminocarbonyl, alkylcarbonyl, cycloalkylcarbonyl, —S(O)H, C₁₋₄-alkylsulfinyl, —S(O)₂H, C₁₋₄ alkylsulfonyl, —S(O)₂NH₂, mono-alkylaminosulfonyl, alkylcarbonylamino, alkylsulfonylamino; preferably Z⁶ is selected from halo, alkyl, haloalkyl, haloalkyloxy, cycloalkyl, cycloalkenyl, aryl, heterocyclyl, heteroaryl, hydroxyl, alkyloxy, cycloalkyloxy, cyano, amino, mono-alkylamino, hydroxycarbonyl, alkyloxycarbonyl, aminocarbonyl, mono-alkylaminocarbonyl, mono-cycloalkylaminocarbonyl, alkylcarbonyl; preferably Z⁶ is selected from halo, alkyl, haloalkyl, cycloalkyl, cycloalkenyl, aryl, heterocyclyl, heteroaryl, hydroxyl, alkyloxy, cyano, amino, mono-alkylamino, aminocarbonyl, mono-cycloalkylaminocarbonyl, alkylcarbonyl.

In some embodiments each Z⁷ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro; preferably Z⁷ is selected from halo, alkyl, haloalkyl, haloalkyloxy, cycloalkyl, cycloalkenyl, aryl, alkylaryl, heterocyclyl, heteroaryl, hydroxyl, alkyloxy, cycloalkyloxy, aryloxy, heterocyclyloxy, heteroaryloxy, cyano, amino, mono-alkylamino, di-alkylamino, mono-cycloalkylamino, di-cycloalkylamino, mono-arylamino, di-arylamino, mono-heterocyclylamino, di-heterocyclylamino, mono-heteroarylamino, di-heteroarylamino, hydroxycarbonyl, alkyloxycarbonyl, cycloalkyloxycarbonyl, aryloxycarbonyl, aminocarbonyl, mono-alkylaminocarbonyl, di-alkylaminocarbonyl, mono-cycloalkylaminocarbonyl, alkylcarbonyl, cycloalkylcarbonyl, arylcarbonyl, —S(O)H, C₁₋₄-alkylsulfinyl, —S(O)₂H, C₁₋₄-alkylsulfonyl, —S(O)₂NH₂, mono-alkylaminosulfonyl, di-alkylaminosulfonyl, alkylcarbonylamino, alkylsulfonylamino, nitro; preferably Z⁷ is selected from halo, alkyl, haloalkyl, haloalkyloxy, cycloalkyl, cycloalkenyl, aryl, alkylaryl, heterocyclyl, heteroaryl, hydroxyl, alkyloxy, cycloalkyloxy, aryloxy, cyano, amino, mono-alkylamino, mono-cycloalkylamino, mono-arylamino, hydroxycarbonyl, alkyloxycarbonyl, cycloalkyloxycarbonyl, aryloxycarbonyl, aminocarbonyl, mono-alkylaminocarbonyl, mono-cycloalkylaminocarbonyl, alkylcarbonyl, cycloalkylcarbonyl, —S(O)H, C₁₋₄-alkylsulfinyl, —S(O)₂H, C₁₋₄alkylsulfonyl, —S(O)₂NH₂, mono-alkylaminosulfonyl, alkylcarbonylamino, alkylsulfonylamino; preferably Z⁷ is selected from halo, alkyl, haloalkyl, haloalkyloxy, cycloalkyl, cycloalkenyl, aryl, heterocyclyl, heteroaryl, hydroxyl, alkyloxy, cycloalkyloxy, cyano, amino, mono-alkylamino, hydroxycarbonyl, alkyloxycarbonyl, aminocarbonyl, mono-alkylaminocarbonyl, mono-cycloalkylaminocarbonyl, alkylcarbonyl; preferably Z⁷ is selected from halo, alkyl, haloalkyl, cycloalkyl, cycloalkenyl, aryl, heterocyclyl, heteroaryl, hydroxyl, alkyloxy, cyano, amino, mono-alkylamino, aminocarbonyl, mono-cycloalkylaminocarbonyl, alkylcarbonyl.

In particular embodiments, these compounds are envisaged for use as a medicament. In further particular embodiments, these compounds are envisaged for use in the prevention or treatment of a disease associated with the activation of receptor tyrosine-protein kinase (ERBB4) as detailed herein.

Particularly preferred compounds of the invention are those compounds listed in Table 1.

TABLE 1

| Compound | Structure |
|---|---|
| 1 | 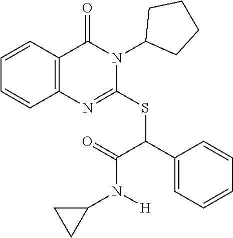 [SMILES: O=C(NC1CC1)C(Sc1nc2ccccc2c(=O)n1C1CCCC1)c1ccccc1] |
| 2 | 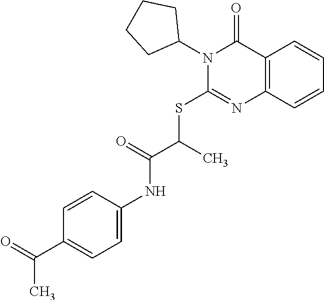 [SMILES: CC(=O)c1ccc(NC(=O)C(C)Sc2nc3ccccc3c(=O)n2C2CCCC2)cc1] |
| 3 | 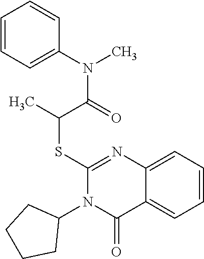 [SMILES: CC(Sc1nc2ccccc2c(=O)n1C1CCCC1)C(=O)N(C)c1ccccc1] |
| 4 | 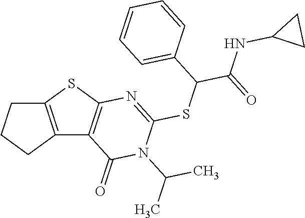 [SMILES: CC(C)n1c(SC(C(=O)NC2CC2)c2ccccc2)nc2sc3c(c2c1=O)CCC3] |
| 5 | 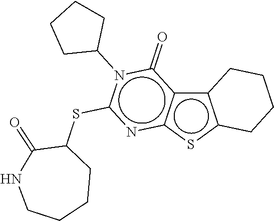 |
| 6 | 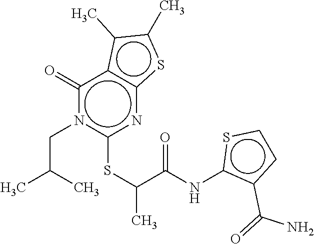 |
| 7 | 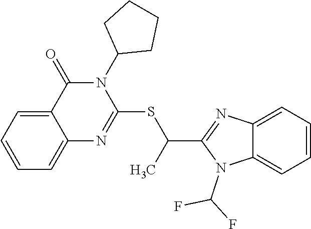 [SMILES: CC(Sc1nc2ccccc2c(=O)n1C1CCCC1)c1nc2ccccc2n1C(F)F] |
| 8 | 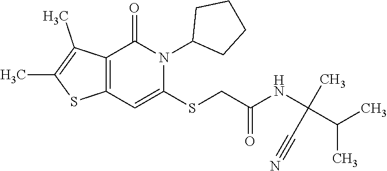 [SMILES: Cc1sc2cc(SCC(=O)NC(C)(C#N)C(C)C)n(C3CCCC3)c(=O)c2c1C] |

The compounds of the present invention have been found to specifically act on the ERBB4 receptor. More particularly, the compounds have been found to be modulators of ERBB4 signalling. Accordingly, the present invention provides compounds of formula (I), and any subgroup thereof such as (IAA), (IA), (IB), (IC), (IIA), (IIB), (IIC), (IID), for use in the prevention or treatment of a disease associated with the activation of receptor tyrosine-protein kinase (ERBB4). In some embodiments the disease associated with the activation of receptor tyrosine-protein kinase (erbB-4) is selected from the group consisting of heart failure, metabolic disorders, inflammatory disorders, fibrotic disorders. Indeed, ERBB4 modulation has been shown to be beneficial in chronic diseases such as chronic heart failure, (chronic) diabetic nephropathy, dermal-, pulmonary- and myocardial fibrosis. More particularly, phase 2 and phase 3 clinical trials are ongoing for the use of neuregulin modulation of erbB4 in the treatment of chronic heart failure. Moreover, the use of neuregulin modulation of ERBB4 in rodents has been shown to decrease skin-, lung-, and myocardial fibrosis, as well as diabetic nephropathy. The present compounds however have the advantage that they are small compounds which do not require intravenous administration, that they are specific for ERBB4, that they are not toxic and do not induce proliferation of cancer cells.

As used herein “heart failure” is a chronic ailment where the heart fails to function normally due to impairment of the hearts pumping ability (left ventricular systolic dysfunction). Heart failure can develop from virtually any cardiac disorder of the pericardium, myocardium, endocardium, or great vessels, but the majority of heart failure patients have symptoms due to the impairment of the left ventricular function. Damage to the left ventricle of the heart limits the ability of the ventricle to fill with blood, or eject blood from the ventricle, resulting in an stiff, thickened, enlarged or, weakened muscle, which can no longer squeeze effectively to pump the blood through the chamber. In some embodiments said heart failure results of an initial inciting influence of ischaemia. In other embodiments said heart failure results of an initial non-ischaemic inciting influence. Heart failure resulting of an initial non-ischaemic inciting influence, such as in the absence of significant coronary artery disease may for example be determined by coronary angiography.

Similarly, the effect of ERBB4 regulation on (chronic) diabetic nephropathy, dermal-, pulmonary- and myocardial fibrosis has been demonstrated in animal models. Accordingly, the present invention also provides the compounds of formula (I), and any subgroup thereof such as (IAA), (IA), (IB), (IC), (IIA), (IIB), (IIC), (IID), for use in the prevention or treatment of chronic diabetic nephropathy, and of fibrotic disorders such as dermal-, pulmonary- and myocardial fibrosis. The present invention also provides the compounds of formula (I), and any subgroup thereof such as (IAA), (IA), (IB), (IC), (IIA), (IIB), (IIC), (IID), for use in the prevention or treatment of (i) heart failure, (ii) lung fibrosis, (iii) skin fibrosis, (iv) liver fibrosis, (v) cardiac rhythm disturbances due to atrial or ventricular fibrosis, (vi) atherosclerotic disease, (vii) diabetic kidney disease (viii) diabetic neuropathy (ix) neurologic disorders, (x) cancer, or (xi) inflammatory bowel disease.

The term “diabetic nephropathy” or “diabetic kidney disease” is used to refer to a disease that is pathologically characterized by glomerular basement membrane (GBM) thickening, glomerular mesangial matrix expansion, and formation of glomerular nodular sclerosis in its advanced stages. Clinically, is defined by proteinuria occurrence or declined renal function, e.g. reduced glomerular filtration rate (GFR). The role of ERBB4 in the maintenance of renal structure has been suggested by the accelerated development of polycystic kidney disease in absence of this receptor. Also, genome-wide association studies (GWAS) of type 1 diabetes-associated nephropathy showed association with an intronic single-nucleotide polymorphism in the ERBB4 gene. Finally, NRG-1 (also acting on ERBB4) has been found to have a nephroprotective effect diabetic mouse models. As used herein, “diabetic neurophathy” refers to a type of nerve damage that can occur in diabetic patients. Diabetic neuropathy is most common in legs and feet. Depending on the affected nerves, diabetic neuropathy symptoms can range from pain and numbness in the legs and feet to problems with the digestive system, urinary tract, blood vessels and heart.

As used herein, “fibrotic disorder” or “fibroproliferative disorder” refers to a pathological condition due to the formation of excess fibrous connective tissue. Non-limiting examples of fibrotic disorders include pulmonary or lung fibroses (such as idiopathic pulmonary fibrosis, chronic fibrosis (or mucoviscidosis)) fibrotic skin disorders (such as systemic sclerosis or scleroderma, and hyperthrophic scarring), liver cirrhosis, progressive kidney disease, and macular degeneration. Pathological fibrosis in the respective tissues or organs is a common hallmark of these disorders “Fibrotic skin disorders” or “fibrotic dermal disorders” are cutaneous disorders characterized by excessive scarring of the skin due to pathologic skin fibrosis. Clinically, skin fibrosis manifests as thickened, tightened, and hardened areas of skin. Ultimately, skin fibrosis may lead to dermal contractures that affect the ability to flex and extend the joints. Non-limiting examples of fibrotic skin disorders include scleroderma in both, localized (morphea, linear scleroderma) and systemic form (scleroderma), hypertrophic scarring, keloids, mixed connective tissue disease, scleredema, scleromyxedema, eosinophilic fasciitis. In preferred embodiments, the fibrotic skin disorder is selected from the group consisting of hypertrophic scarring, and sclerosis, in particular systemic sclerosis (or scleroderma).

A “pulmonary fibrosis” or “fibrotic lung disorder” is a respiratory disease in which scars are formed in the lung tissues, leading to serious breathing problems. Diseases which are primarily characterized by fibrosis in the lung are also referred to as interstitial lung diseases. Symptoms of pulmonary fibroses are mainly: shortness of breath, particularly with exertion, chronic dry, hacking coughing, fatigue and weakness, chest discomfort including chest pain, and loss of appetite and rapid weight loss. In preferred embodiments, the fibrotic lung disorder is idiopathic pulmonary fibrosis (IPF).

“Liver cirrhosis” or “liver fibrosis” is a slowly progressing disease in which healthy liver tissue is replaced with scar tissue, thereby preventing the liver from functioning properly. The scar tissue blocks the flow of blood through the liver and thereby slows the processing of nutrients, hormones, drugs, and naturally produced toxins. It also slows the production of proteins and other substances made by the liver. Liver cirrhosis may cause a wide range of symptoms, including tendency to bleed or bruise early, fatigue, jaundice or yellowing of the skin and eyes, ascites or fluid build-up in the abdomen, weight loss, itchy skin, nausea, swelling in the legs, disorientation and drowsiness, slurred speeh and development of spider-like vessels underneath the skin surface.

Myocardial fibrosis is defined as an increased quantity of collagenous scar tissue in the heart (affecting atria or ventricules or both). Myocardial fibrosis may arise as a result of cardiac disease and/or extracardiac diseases and results in cardiac rhythm disturbances.

As used herein “atherosclerotic disease” refers to the lesions and abnormalities on the arteries walls. These lesions may lead to narrowing due to the buildup or plaque, comprising fats cholesterol and other substances. When severe, it can result in coronary artery disease, stroke, peripheral artery disease, or kidney problems, depending on which arteries are affected.

In line with the reported involvement of the ERBB4 receptor in metabolic disorders and inflammatory disorders, the present invention also provides the compounds of formula (I), and any subgroup thereof such as (IAA), (IA), (IB), (IC), (IIA), (IIB), (IIC), (IID), for use in the prevention or treatment of metabolic and inflammatory disorders.

As used herein, a “metabolic disorder” means any disorder associated with metabolism, and examples include but are not limited to, obesity, central obesity, insulin resistance, glucose intolerance, abnormal glycogen metabolism, type 2 diabetes, hyperlipidemia, hypoalbuminemia, hypertriglyceridemia, metabolic syndrome, syndrome X, a fatty liver, fatty liver disease, polycystic ovarian syndrome, and acanthosis nigricans.

As used herein “inflammatory disorder” denotes a condition of sustained or chronic inflammation that occurs when tissues are injured by viruses, bacteria, trauma, chemicals, heat, cold or any other harmful stimulus. In some embodiments the inflammatory disorder according to the invention selected from the group comprising gastrointestinal inflammatory disorder, skin inflammatory disorders and multiple sclerosis

In some embodiments the gastrointestinal inflammatory disorder is selected from the group consisting of an inflammatory bowel disease (IBD) such as Irritable Bowel Syndrome (IBS), ulcerative colitis and Crohn's disease, an ulcer resulting from administration of a non-steroidal anti-inflammatory drug, such as a peptic ulcer (i.e. a sore that forms in the lining of the stomach or the duodenum), and an inflammatory disorder associated with an infection with Schistosoma mansoni parasite. In some embodiments the skin inflammatory disorder is selected from the group consisting of eczema, dermatitis, including for example, atopic dermatitis, seborrheic dermatitis, dyshidrotic eczema, nummular dermatitis, stasis dermatitis, allergic dermatitis, psoriasis, pruritis, multiple sclerosis, cutaneous inflammation, cicatricial pemphigoid, scleroderma, hidradenitis suppurativa, toxic epidermal necrolysis, acne, osteitis, graft vs. host disease (GvHD), pyroderma gangrenosum, and Behcet's Syndrome.

As used herein “neurological disorder” refers to a condition having as a component a central or peripheral nervous system malfunction. Neurological disorders may cause a disturbance in the structure or function of the nervous system resulting from developmental abnormalities, disease, genetic defects, injury or toxin. These disorders may affect the central nervous system (e.g., the brain, brainstem and cerebellum), the peripheral nervous system (e.g., the cranial nerves, spinal nerves, and sympathetic and parasympathetic nervous systems) and/or the autonomic nervous system (e.g., the part of the nervous system that regulates involuntary action and that is divided into the sympathetic and parasympathetic nervous systems).

In some instances the neurological disorder is a neuropsychiatric disorder, which refers to conditions or disorders that relate to the functioning of the brain and the cognitive processes or behavior. Some neuropsychiatric disorders include disorders in the schizophrenia spectrum and other psychotic disorders, depressive disorders, and anxiety disorders.

In some instances, the neurological disorder is a disorder involving demyelination (and/or insufficient remyelination) of the CNS such as a result of autoimmune disease, genetic mutation, or trauma such as injury or stroke. In particular embodiment the disease or disorder is multiple sclerosis.

In particular embodiments the compound of the invention is envisaged for use in the treatment of (i) heart failure, (ii) lung fibrosis, (iii) skin fibrosis (sclerodermia, systemic sclerosis), (iv) liver fibrosis, (v) cardiac rhythm disturbances due to atrial or ventricular fibrosis, (vi) atherosclerotic disease, (vii) diabetic kidney disease (viii) diabetic neuropathy (ix) neurologic disorders (schizophrenia spectrum and other psychotic disorders, depressive disorders, multiple sclerosis, and anxiety disorders), (x) forms of cancer in which ERBB4 activation has been shown to be protective (such as but not limited to mammary gland tumors, colorectal cancer, hepatocellular carcinoma), (xi) inflammatory bowel disease.

ERBB4 has been found to be over-expressed in different types of cancer and high level expression of ERBB4 has been associated with poor prognosis.

Accordingly, the present invention provides compounds of formula (I), and any subgroup thereof such as (IAA), (IA), (IB), (IC), (IIA), (IIB), (IIC), (IID), for use in the prevention or treatment of cancer. More particularly, the invention provides the use of these compounds in the treatment of tumors with high ERBB4 receptor levels.

As used herein the term “cancer” means various malignant tumors originated from epithelial cells in various tissues, cells such as colon cancer, lung cancer, liver cancer, gastric cancer, renal cancer, gallbladder cancer, prostate cancer, pancreatic cancer, testis cancer, ovarian cancer, cutaneous cancer, esophagus cancer, laryngeal cancer, breast cancer or uterine cancer. In particular, forms of cancer in which ERBB4 activation has been shown to be protective include mammary gland tumors, colorectal cancer, hepatocellular carcinoma.

A further and related aspect of the invention relates to methods of treatment of a disease associated with the activation of receptor tyrosine-protein kinase (ERBB4) which involve administrating compounds of formula (I) or any subgroup thereof as described herein to a subject in need thereof.

In some embodiments the disease associated with the activation of receptor tyrosine-protein kinase (ERBB-4) is selected from the group consisting of (i) heart failure, (ii) lung fibrosis, (iii) skin fibrosis such as sclerodermia or systemic sclerosis, (iv) liver fibrosis, (v) cardiac rhythm disturbances due to atrial or ventricular fibrosis, (vi) atherosclerotic disease, (vii) diabetic kidney disease (viii) diabetic neuropathy (ix) neurologic disorders such as schizophrenia spectrum and other psychotic disorders, depressive disorders, multiple sclerosis, and anxiety disorders, (x) cancer, or (xi) inflammatory bowel disease.

The compounds of the invention may be in the form of salts, preferably pharmaceutically acceptable salts, as generally described below. Some preferred, but non-limiting examples of suitable pharmaceutically acceptable organic and/or inorganic acids are as hydrochloric acid, hydrobromic acid, sulfuric acid, nitric acid, acetic acid and citric acid, as well as other pharmaceutically acceptable acids known per se (for which reference is made to the prior art referred to below).

When the compounds of the invention contain an acidic group as well as a basic group the compounds of the invention may also form internal salts, and such compounds are within the scope of the invention. When the compounds of the invention contain a hydrogen-donating heteroatom (e.g. NH), the invention also covers salts and/or isomers formed by transfer of said hydrogen atom to a basic group or atom within the molecule.

Pharmaceutically acceptable salts of the compounds of formula (I) and any subgroup thereof include the acid addition and base salts thereof. Suitable acid addition salts are formed from acids which form non-toxic salts. Examples include the acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts. For a review on suitable salts, see Handbook of Pharmaceutical Salts: Properties, Selection, and Use by Stahl and Wermuth (Wiley-VCH, 2002), incorporated herein by reference.

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterized by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

Pharmaceutically acceptable salts of compounds of formula (I) may be prepared by one or more of these methods:
(i) by reacting the compound of formula (I) with the desired acid;
(ii) by reacting the compound of formula (I) with the desired base;
(iii) by removing an acid- or base-labile protecting group from a suitable precursor of the compound of formula (I) or by ring-opening a suitable cyclic precursor, for example, a lactone or lactam, using the desired acid; or
(iv) by converting one salt of the compound of formula (I) to another by reaction with an appropriate acid or by means of a suitable ion exchange column.

All these reactions are typically carried out in solution. The salt may precipitate from solution and be collected by filtration or may be recovered by evaporation of the solvent. The degree of ionization in the salt may vary from completely ionized to almost non-ionized.

The compounds of the invention may also exist in unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water.

A currently accepted classification system for organic hydrates is one that defines isolated site, channel, or metal-ion coordinated hydrates—see Polymorphism in Pharmaceutical Solids by K. R. Morris (Ed. H. G. Britain, Marcel Dekker, 1995), incorporated herein by reference. Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

Also included within the scope of the invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallization, by recrystallization from solvents, or by physically grinding the components together—see Chem Commun, 17, 1889-1896, by O. Almarsson and M. J. Zaworotko (2004), incorporated herein by reference. For a general review of multi-component complexes, see J Pharm Sci, 64 (8), 1269-1288, by Haleblian (August 1975), incorporated herein by reference.

The compounds of the invention may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970), incorporated herein by reference.

All references to compounds of formula (I) or any subgroups thereof include references to salts, solvates, multi-component complexes and liquid crystals thereof and to solvates, multi-component complexes and liquid crystals of salts thereof.

The compounds of the invention include compounds of formula (I) or any subgroups thereof as hereinbefore defined, including all polymorphs and crystal habits thereof, prodrugs and isomers thereof (including optical, geometric and tautomeric isomers) as hereinafter defined and isotopically-labeled compounds of formula (I).

In addition, although generally, with respect to the salts of the compounds of the invention, pharmaceutically acceptable salts are preferred, it should be noted that the invention in its broadest sense also included non-pharmaceutically acceptable salts, which may for example be used in the isolation and/or purification of the compounds of the invention.

A further aspect of the present invention provides a pharmaceutical composition comprising a compound of formula (I) or a stereoisomer, or tautomer thereof,

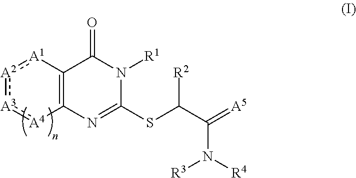

wherein,
each dotted line represents an optional double bond whereby maximally 2 non-adjacent dotted lines can form a double bond;
n is an integer selected from 0, 1, or 2;
A¹ is selected from the group consisting of CR⁵, S, N, NR⁶, and O;
A² is selected from the group consisting of CR⁵, S, N, NR⁶, and O;
A³ is selected from the group consisting of CR⁵, S, N, NR⁶, and O;
A⁴ is selected from the group consisting of CR⁵, S, N, NR⁶, and O;
wherein at most two of A¹ to A⁴ is selected from S, N, NR⁶, and O;
A⁵ is 0 or NR⁷;
R¹ is selected from the group consisting of alkyl, cycloalkyl, alkenyl, aryl, arylalkyl, heterocyclyl, and heteroaryl; wherein said alkyl, cycloalkyl, alkenyl, aryl, arylalkyl, heterocyclyl, or heteroaryl can be unsubstituted or substituted with one or more Z¹;
R² is selected from the group consisting of hydrogen, alkyl, cycloalkyl, aryl, arylalkyl, heterocyclyl, and heteroaryl; wherein said alkyl, cycloalkyl, aryl, arylalkyl, heterocyclyl, or heteroaryl can be unsubstituted or substituted with one or more Z²;
R³ is selected from the group consisting of hydrogen, cycloalkyl, aryl, alkyl, haloalkyl, haloalkyloxy, arylalkyl, heterocyclyl, heteroaryl, —C(O)R⁸, —CO₂R⁹, —C(O)NR⁹R¹⁰, —S(O)₂R⁹, and —SO₂NR⁹R¹⁰;
wherein said cycloalkyl, aryl, alkyl, arylalkyl, heterocyclyl, or heteroaryl can be unsubstituted or substituted with one or more Z³;
or R² and R³ together with the atom to which they are attached form a saturated or unsaturated 5-, 6-, 7-, 8- or 9-membered ring;
R⁴ is selected from the group consisting of hydrogen, cycloalkyl, aryl, alkyl, haloalkyl, haloalkyloxy, arylalkyl, heterocyclyl, and heteroaryl; wherein said cycloalkyl, aryl, alkyl, haloalkyl, haloalkyloxy, arylalkyl, heterocyclyl, or heteroaryl can be unsubstituted or substituted with one or more Z⁴;
or R³ and R⁴ together with the nitrogen atom to which they are attached form a saturated or unsaturated 5-, 6-, 7-, 8-, 9-, 10- or 11-membered nitrogen-containing monocycle or bicycle;
R⁵ is selected from the group consisting of hydrogen, alkyl, cycloalkyl, halo, aryl, arylalkyl, heterocyclyl, heteroaryl, hydroxyl, —OR^{B}, cyano, amino, nitro, haloalkyl, haloalkyloxy, —C(O)R⁸, —NR⁸R⁹, —CO₂R⁹, —C(O)NR⁹R¹⁰, —S(O)₂R⁹, —S(O)₂NR⁹R¹⁰, —NR⁹C(O)_{R}¹⁰ and —NR⁹S(O)₂R¹⁰;
or wherein two R⁵ together with the atom to which they are attached can form a saturated or unsaturated 5-, 6-, 7-, 8-, 9- or 10-membered ring;
wherein said ring, alkyl, cycloalkyl, aryl, arylalkyl, heterocyclyl, or heteroaryl can be unsubstituted or substituted with one or more Z⁵;
R⁶ is selected from the group consisting of hydrogen, alkyl, cycloalkyl, aryl, arylalkyl, heterocyclyl, heteroaryl, and —S(O)₂R⁹; wherein said alkyl, cycloalkyl, aryl, arylalkyl, heterocyclyl, or heteroaryl can be unsubstituted or substituted with one or more Z⁶;
R⁷ is selected from the group consisting of alkyl, cycloalkyl, aryl, arylalkyl, heterocyclyl, and heteroaryl; wherein said alkyl, cycloalkyl, aryl, arylalkyl, heterocyclyl, or heteroaryl can be unsubstituted or substituted with one or more Z⁷;
or R³ and R⁷ together with the atom to which they are attached form an unsaturated 5-, 6-, 7-, 8-, 9-, 10- or 11-membered nitrogen-containing monocycle or bicycle;
each R⁸ is independently selected from the group consisting of, alkyl, aryl, cycloalkyl, arylalkyl, heterocyclyl, heteroaryl;
each R⁹ is independently selected from the group consisting of hydrogen, alkyl, aryl, cycloalkyl, arylalkyl, heterocyclyl, heteroaryl;
each R¹⁰ is independently selected from the group consisting of hydrogen, alkyl, aryl, cycloalkyl, arylalkyl, heterocyclyl, heteroaryl;
each Z¹ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z² is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z³ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z⁴ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z⁵ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z⁶ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z⁷ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro; and a pharmaceutically acceptable carrier.

The term “pharmaceutically acceptable” as used herein is consistent with the art and means compatible with the other ingredients of a pharmaceutical composition and not deleterious to the recipient thereof.

As used herein, “carrier” or “excipient” includes any and all solvents, diluents, buffers (such as, e.g., neutral buffered saline or phosphate buffered saline), solubilisers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives, antioxidants, tonicity controlling agents, absorption delaying agents, and the like. The use of such media and agents for pharmaceutical active substances is well known in the art. Except insofar as any conventional media or agent is incompatible with the active substance, its use in the therapeutic compositions may be contemplated.

Illustrative, non-limiting carriers for use in formulating the pharmaceutical compositions include, for example, oil-in-water or water-in-oil emulsions, aqueous compositions with or without inclusion of organic co-solvents suitable for intravenous (IV) use, liposomes or surfactant-containing vesicles, microspheres, microbeads and microsomes, powders, tablets, capsules, suppositories, aqueous suspensions, aerosols, and other carriers apparent to one of ordinary skill in the art. Pharmaceutical compositions as intended herein may be formulated for essentially any route of administration, such as without limitation, oral administration (such as, e.g., oral ingestion or inhalation), intranasal administration (such as, e.g., intranasal inhalation or intranasal mucosal application), parenteral administration (such as, e.g., subcutaneous, intravenous (I.V.), intramuscular, intraperitoneal or intrasternal injection or infusion), transdermal or transmucosal (such as, e.g., oral, sublingual, intranasal) administration, topical administration, rectal, vaginal or intra-tracheal instillation, and the like. In this way, the therapeutic effects attainable by the methods and compositions can be, for example, systemic, local, tissue-specific, etc., depending of the specific needs of a given application.

In preferred embodiments, the compound or the pharmaceutical composition as taught herein is administered parenterally. More preferably, the compound or the pharmaceutical composition as taught herein is administered intravenously, for example by infusion.

The dosage or amount of the agent as taught herein, optionally in combination with one or more other active compounds to be administered, depends on the individual case and is, as is customary, to be adapted to the individual circumstances to achieve an optimum effect. Thus, the unit dose and regimen depend on the nature and the severity of the disorder to be treated, and also on factors such as the species of the subject, the sex, age, body weight, general health, diet, mode and time of administration, immune status, and individual responsiveness of the human or animal to be treated, efficacy, metabolic stability and duration of action of the compounds used, on whether the therapy is acute or chronic or prophylactic, or on whether other active compounds are administered in addition to the agent of the invention. In order to optimize therapeutic efficacy, the compound or the pharmaceutical composition as taught herein can be first administered at different dosing regimens. Typically, levels of the agent in a tissue can be monitored using appropriate screening assays as part of a clinical testing procedure, e.g., to determine the efficacy of a given treatment regimen. The frequency of dosing is within the skills and clinical judgement of medical practitioners (e.g., doctors, veterinarians or nurses). Typically, the administration regime is established by clinical trials which may establish optimal administration parameters. However, the practitioner may vary such administration regimes according to the one or more of the aforementioned factors, e.g., subject's age, health, weight, sex and medical status. The frequency of dosing can be varied depending on whether the treatment is prophylactic or therapeutic.

Toxicity and therapeutic efficacy of the agent as described herein or pharmaceutical compositions comprising the same can be determined by known pharmaceutical procedures in, for example, cell cultures or experimental animals. These procedures can be used, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Pharmaceutical compositions that exhibit high therapeutic indices are preferred. While pharmaceutical compositions that exhibit toxic side effects can be used, care should be taken to design a delivery system that targets such compounds to the site of affected tissue in order to minimize potential damage to normal cells (e.g., non-target cells) and, thereby, reduce side effects.

The data obtained from the cell culture assays and animal studies can be used in formulating a range of dosage for use in appropriate subjects. The dosage of such pharmaceutical compositions lies generally within a range of circulating concentrations that include the ED50 with little or no toxicity. The dosage may vary within this range depending upon the dosage form employed and the route of administration utilised. For a pharmaceutical composition used as described herein, the therapeutically effective dose can be estimated initially from cell culture assays. A dose can be formulated in animal models to achieve a circulating plasma concentration range that includes the IC50 (i.e., the concentration of the pharmaceutical composition which achieves a half-maximal inhibition of symptoms) as determined in cell culture. Such information can be used to more accurately determine useful doses in humans. Levels in plasma can be measured, for example, by high performance liquid chromatography.

In particular embodiment, the compound as taught herein is the main or only active ingredient of the pharmaceutical composition.

According to particular embodiments, the present invention provides a pharmaceutical composition comprising a compound of formula (I), wherein,
each Z¹ is independently selected from the group consisting of halo, C₁₋₆alkyl, haloC₁₋₄alkyl, haloC₁₋₄alkyloxy, C₃₋₁₂cycloalkyl, C₃₋₁₂cycloalkenyl, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, mono-C₁₋₄alkylamino, mono-C₃₋₁₂cycloalkylamino, mono-C₆₋₁₂arylamino, hydroxycarbonyl, C₁₋₄alkyloxycarbonyl, C₃₋₁₂cycloalkyloxycarbonyl, C₆₋₁₂aryloxycarbonyl, aminocarbonyl, mono-C₁₋₄alkylaminocarbonyl, mono-C₃₋₁₂cycloalkylaminocarbonyl, C₁₋₄alkylcarbonyl, C₃₋₁₂cycloalkylcarbonyl, C₆₋₁₂arylcarbonyl, —S(O)H, C₁₋₄alkylsulfinyl, —S(O)₂H, C₁₋₄alkylsulfonyl, —SO₂NH₂, mono-C₁₋₄alkylaminosulfonyl, nitro;
each Z² is independently selected from the group consisting of halo, C₁₋₆alkyl, haloC₁₋₄alkyl, haloC₁₋₄alkyloxy, C₃₋₁₂cycloalkyl, C₃₋₁₂cycloalkenyl, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, mono-C₁₋₄alkylamino, mono-C₃₋₁₂cycloalkylamino, mono-C₆₋₁₂arylamino, hydroxycarbonyl, C₁₋₄alkyloxycarbonyl, C₃₋₁₂cycloalkyloxycarbonyl, C₆₋₁₂aryloxycarbonyl, aminocarbonyl, mono-C₁₋₄alkylaminocarbonyl, mono-C₃₋₁₂cycloalkylaminocarbonyl, C₁₋₄alkylcarbonyl, C₃₋₁₂cycloalkylcarbonyl, C₆₋₁₂arylcarbonyl, —S(O)H, C₁₋₄alkylsulfinyl, —S(O)₂H, C₁₋₄alkylsulfonyl, —SO₂NH₂, mono-C₁₋₄alkylaminosulfonyl, nitro;
each Z³ is independently selected from the group consisting of halo, C₁₋₆alkyl, haloC₁₋₄alkyl, haloC₁₋₄alkyloxy, C₃₋₁₂cycloalkyl, C₃₋₁₂cycloalkenyl, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, mono-C₁₋₄akylamino, mono-C₃₋₁₂cycloalkylamino, mono-C₆₋₁₂arylamino, hydroxycarbonyl, C₁₋₄alkyloxycarbonyl, C₃₋₁₂cycloalkyloxycarbonyl, C₆₋₁₂aryloxycarbonyl, aminocarbonyl, mono-C₁₋₄alkylaminocarbonyl, mono-C₃₋₁₂cycloalkylaminocarbonyl, C₁₋₄alkylcarbonyl, C₃₋₁₂cycloalkylcarbonyl, C₆₋₁₂arylcarbonyl, —S(O)H, C₁₋₄alkylsulfinyl, —S(O)₂H, C₁₋₄alkylsulfonyl, —SO₂NH₂, mono-C₁₋₄alkylaminosulfonyl, nitro;
each Z⁴ is independently selected from the group consisting of halo, C₁₋₆alkyl, haloC₁₋₄alkyl, haloC₁₋₄alkyloxy, C₃₋₁₂cycloalkyl, C₃₋₁₂cycloalkenyl, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, mono-C₁₋₄alkylamino, mono-C₃₋₁₂cycloalkylamino, mono-C₆₋₁₂arylamino, hydroxycarbonyl, C₁₋₄alkyloxycarbonyl, C₃₋₁₂cycloalkyloxycarbonyl, C₆₋₁₂aryloxycarbonyl, aminocarbonyl, mono-C₁₋₄alkylaminocarbonyl, mono-C₃₋₁₂cycloalkylaminocarbonyl, C₁₋₄alkylcarbonyl, C₃₋₁₂cycloalkylcarbonyl, C₆₋₁₂arylcarbonyl, —S(O)H, C₁₋₄alkylsulfinyl, —S(O)₂H, C₁₋₄alkylsulfonyl, —SO₂NH₂, mono-C₁₋₄alkylaminosulfonyl, nitro;
each Z⁵ is independently selected from the group consisting of halo, C₁₋₆alkyl, haloC₁₋₄alkyl, haloC₁₋₄alkyloxy, C₃₋₁₂cycloalkyl, C₃₋₁₂cycloalkenyl, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, mono-C₁₋₄akylamino, mono-C₃₋₁₂cycloalkylamino, mono-C₆₋₁₂arylamino, hydroxycarbonyl, C₁₋₄-alkyloxycarbonyl, C₃₋₁₂cycloalkyloxycarbonyl, C₆₋₁₂aryloxycarbonyl, aminocarbonyl, mono-C₁₋₄alkylaminocarbonyl, mono-C₃₋₁₂cycloalkylaminocarbonyl, C₁₋₄alkylcarbonyl, C₃₋₁₂cycloalkylcarbonyl, C₆₋₁₂arylcarbonyl, —S(O)H, C₁₋₄-alkylsulfinyl, —S(O)₂H, C₁₋₄alkylsulfonyl, —SO₂NH₂, mono-C₁₋₄alkylaminosulfonyl, nitro;
each Z⁶ is independently selected from the group consisting of halo, C₁₋₆alkyl, haloC₁₋₄alkyl, haloC₁₋₄alkyloxy, C₃₋₁₂cycloalkyl, C₃₋₁₂cycloalkenyl, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, mono-C₁₋₄akylamino, mono-C₃₋₁₂cycloalkylamino, mono-C₆₋₁₂arylamino, hydroxycarbonyl, C₁₋₄-alkyloxycarbonyl, C₃₋₁₂cycloalkyloxycarbonyl, C₆₋₁₂aryloxycarbonyl, aminocarbonyl, mono-C₁₋₄alkylaminocarbonyl, mono-C₃₋₁₂cycloalkylaminocarbonyl, C₁₋₄alkylcarbonyl, C₃₋₁₂cycloalkylcarbonyl, C₆₋₁₂arylcarbonyl, —S(O)H, C₁₋₄-alkylsulfinyl, —S(O)₂H, C₁₋₄alkylsulfonyl, —SO₂NH₂, mono-C₁₋₄alkylaminosulfonyl, nitro;
each Z⁷ is independently selected from the group consisting of halo, C₁₋₆alkyl, haloC₁₋₄alkyl, haloC₁₋₄alkyloxy, C₃₋₁₂cycloalkyl, C₃₋₁₂cycloalkenyl, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, mono-C₁₋₄alkylamino, mono-C₃₋₁₂cycloalkylamino, mono-C₆₋₁₂arylamino, hydroxycarbonyl, C₁₋₄alkyloxycarbonyl, C₃₋₁₂cycloalkyloxycarbonyl, C₆₋₁₂aryloxycarbonyl, aminocarbonyl, mono-C₁₋₄alkylaminocarbonyl, mono-C₃₋₁₂cycloalkylaminocarbonyl, C₁₋₄alkylcarbonyl, C₃₋₁₂cycloalkylcarbonyl, C₆₋₁₂arylcarbonyl, —S(O)H, C₁₋₄alkylsulfinyl, —S(O)₂H, C₁₋₄alkylsulfonyl, —SO₂N H₂, mono-C₁₋₄alkylaminosulfonyl, nitro;
and a pharmaceutically acceptable carrier.

### EXAMPLES

The following examples are provided for the purpose of illustrating the present invention and by no means should be interpreted to limit the scope of the present invention.

Cells

T47D, EFO-21, MCF7 and HDF cells were cultured in Dulbecco's modified Eagle's medium (DMEM, ThermoFisher Scientific, 11995065) supplemented with 10% (v/v) heat-inactivated fetal bovine serum (FBS, ThermoFisher Scientific, 10082147), 1% penicillin/streptomycin and 1 mL polymyxin B and were maintained at 37° C. in humidified atmosphere of 5% CO2 in air.

U2OS ERBB4/ERBB4 dimerization cells were cultured according to the manufacturer's instructions. Briefly, cells were thawed and cultured in Thawing Reagent T3 (92-4103TR, DiscoverX) up and including the first propagation. Afterwards cells were cultured using Cell Culture Reagent 103 (92-3103G, DiscoverX) supplemented with 250 μg/mL Hygromycin B and 500 μg/mL G418 (DiscoverX) and were maintained at 37° C. in humidified atmosphere of 5% CO2 in air.

High Throughput Screening

Following 1-12 passages, U2OS ERBB4/ERBB4 dimerization cells were seeded at 10,000 cells/well in 100 μL cell plating reagent in white walled 384-well plates (Greiner Bio-One, 781080). Cells were treated with compound (10 μM, Pharmacological Diversity Set, Enamine), Heregulin-β1 (1 μM, DiscoverX, 92-1031) or H₂O. All wells had a final concentration of 1% DMSO. Cells were subsequently incubated for 6 hours at 37° C., 5% CO2. Then 25 μL PathHunter Flash Detection Reagents (93-0247) was added to each well and cells were incubated at room temperature for 1 hour (covered from light). Afterwards luminescence signal was measured using the EnVision plate reader (Perkin Elmer)

Software

Spotfire (TIBCO) was used for data visualization, CDD Vault (Collaborative Drug Discovery) for compound registry and data management, Columbus (PerkinElmer) for data analysis and data storage.

Based on the results obtained, 8 compounds were selected for further characterization.

General Protocol Dose-Response Curves

PathHunter® Dimerization Assay in 96-Well Format

Following 1-12 passages, U2OS ERBB4/ERBB4 dimerization cell line (Eurofins, 93-0961C₃) were seeded at 10,000 cells/well in 100 μL cell plating reagent (Eurofins, 93-0563R0A) in white walled 96-well plates (PerkinElmer, 6005680) and incubated for 24h at 37° C. in humidified atmosphere of 5% CO2 in air. Cells were treated with 10 μL of compound (prepared at 11×, Enamine), 10 μM Heregulin-β1 (NRG-1) (DiscoverX, 92-1031 or Peprotech, 100-03) or PBS. All wells had a final concentration of 0,9% DMSO. Cells were subsequently incubated for 6 hours at 37° C., 5% CO2. Then 110 μL PathHunter Flash Detection Reagents (93-0247) were added to each well and cells were incubated at room temperature for 1 hour (covered from light). Afterwards luminescence signal was measured using the Luminoskan Ascent (Thermofisher). Dose response curves of the compounds were performed in 2-fold (0,0625 μM-32 μM).

From these curves, the EC₅₀ and the Eₘₐₓ of the compounds was calculated. Table 2 shows the resulting values.

TABLE 2

| Compound | EC₅₀ (M) | Eₘₐₓ (RLU (%)) |
|---|---|---|
| NRG-1 | (3.80 ± 1.09) *10⁻⁸ | 100 |
| 1 | (8.92 ± 1.63) *10⁻⁶ | 33.06 ± 4.21 |
| 2 | (10.87 ± 2.96) *10⁻⁶ | 9.48 ± 2.13 |
| 3 | (15.75 ± 1.14) *10⁻⁶ | 14.32 ± 2.63 |
| 4 | (7.54 ± 1.47) *10⁻⁶ | 16.91 ± 3.69 |
| 5 | (6.11 ± 1.20) *10⁻⁶ | 17.69 ± 3.45 |
| 6 | (6.47 ± 1.12) *10⁻⁶ | 10.34 ± 3.01 |
| 7 | (1039 ± 1.69) *10⁻⁶ | 9.76 ± 2.46 |
| 8 | (20.40 ± 1.85) *10⁻⁶ | 18.45 ± 3.47 |

RLU = relative light units

Identification of Modulators

Cells were treated with compound (prepared at 22×, 10 min pre-incubation) together with NRG-1 (0.1 μM final concentration, Peprotech, 100-03), or NRG-1 (0.1 μM final concentration) or PBS. All wells had a final concentration of 0.9% DMSO.

Software

GraphPad Prism version 7.0 (GraphPad Software) was used for data visualization.

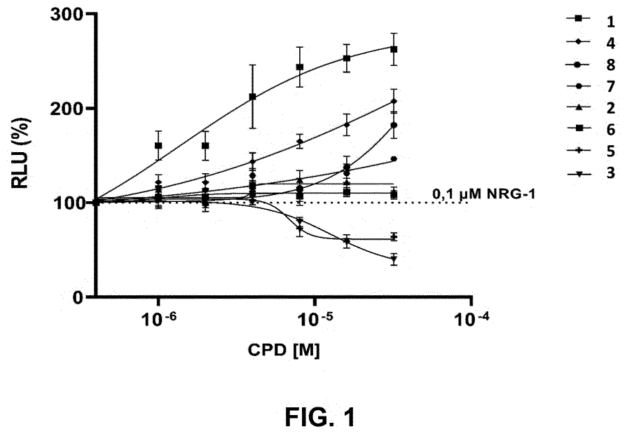

FIG. 1 shows the dose-response curves of the compounds on top of 0.1 μM neuregulin-1 (NRG1). From the shape of the curves, compounds 3 and 5 seem to behave as partial agonists or allosteric agonists functioning as partial agonists. Compounds 1, 4 and 8 seem to be ago-allosteric modulators. Compounds 2, 6 and 7 seem to be weak or silent allosteric modulators. The EC₅₀ and the Eₘₐₓ of the compounds on top of 0.1 μM NRG1 is depicted in Table 3 relative to NRG1 (set as 100%).

TABLE 3

| Compound | EC50 (M) | Emax (RLU (%)) |
|---|---|---|
| 1 | ~(1.60 ± 2.29) * 10⁻⁶ | 256 ± 126 |
| 2 | / | 126 ± 36 |
| 3 | / | 36 ± 13 |
| 4 | ~(21.5 ± 15.5) *10⁻⁶ | 192 ± 34 |
| 5 | ~(3.36 ± 3.05) * 10⁻⁶ | 63 ± 14 |
| 6 | ~(13.9 ± 15.6) *10⁻⁶ | 112 ± 14 |
| 7 | / | 139 ± 19 |
| 8 | / | 174 ± 38 |

RLU = relative light units

Competition Binding Assay

Fluorescent Labeling of NRG-1

NRG-1 (Peprotech, 100-03) was dissolved in PBS at a concentration of 1 mg/mL and labelled with the Alexa Fluor® 488 using the Alexa Fluor® 488 microscale protein labelling kit (A30006, Thermofisher). A Bio-gel P-4 (Bio-rad, 1504124) fine resin suspended in PBS and a dye:protein molar ratio (MR) of 5 was used to perform the labelling according to the manufacturer's instructions.

Competition Binding Assay

U2OS ERBB4/ERBB4 dimerization cell line (Eurofins, 93-0961C₃) was used to perform a competition binding assay with fluorescent labelled NRG-1 using flow cytometry. Cells were rinsed with ice-cold PBS, tyrpsinized (0.25%) and seeded in transparent U-bottom 96-well plates (Greiner CELLSTAR®, M9436) at 0.5 million cells/mL in 50 μL ice cold buffer (PBS with 0,1% Bovine Serum Albumin and 0,05% Sodium Azide). Next, cells were centrifugated at 1400 rpm for 4 minutes and supernatant was discarded. Cell pellet was washed with 50 μL ice cold buffer by gently pipetting up and down. Centrifugation and washing once repeated once more and supernatant was discarded. Next, cells were treated with compound (pre-incubation of 10 min on ice) and 30 nM fluorescent labelled NRG-1 (F-NRG), 30 nM F-NRG-1 (positive control), buffer (unstained) or 30 nM F-NRG with 1500 nM NRG-1 (aspecific binding control). All wells had a final concentration of 0.9% DMSO. After gently pipetting up and down, the plate was incubated for 1 h at 4° C. on a plate-shaker. After incubation the plate was centrifuged at 1400 rpm for 4 min and supernatant was discarded. Next, the cells were washed with 100 μL ice-cold buffer and centrifuged and washed again. Supernatant was discarded and 100 μL ice-cold buffer was added to the pellet and pipetted up and down to have a single cell suspension. The cell solution was then transferred to a 5 mL polystyrene round-bottom tube. Samples were kept on ice and exposed to minimum light when proceeded to flow cytometry (BD Accuri C6, BD Biosciences). Unstained cells were used to set the parameters of the flow cytometer.

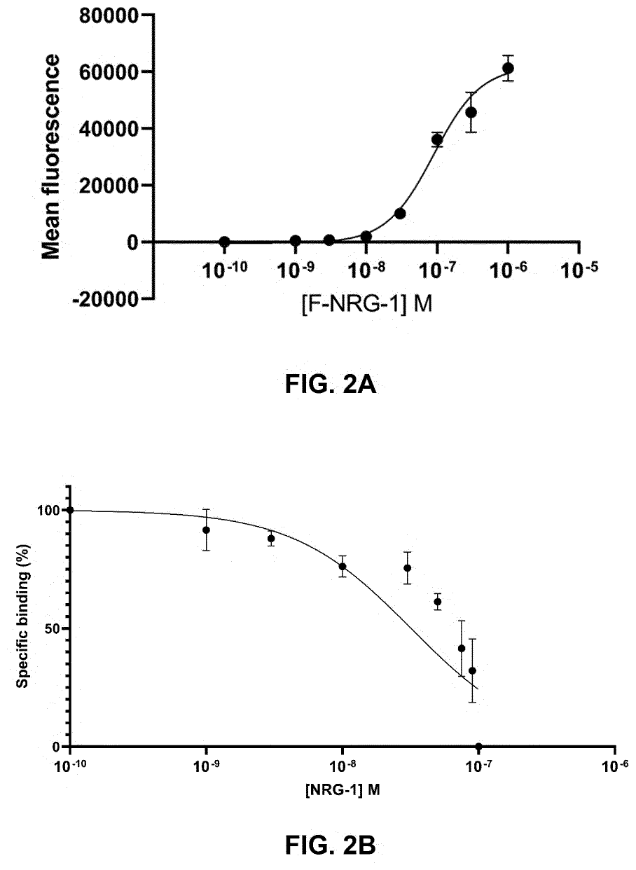

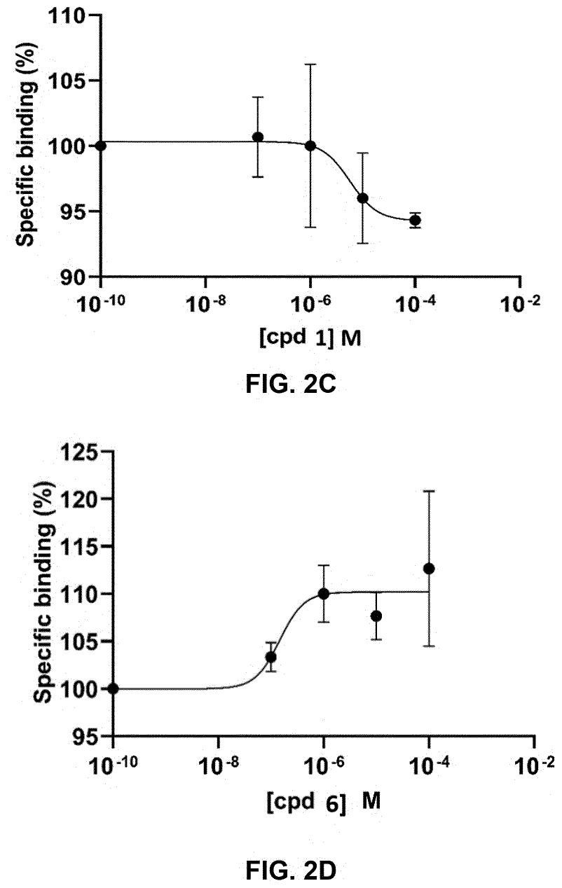

FIG. 2A shows the dose-response curve of F-NRG-1, with an EC₅₀ of 8.8*10⁻⁸ M. FIG. 2B shows dose-response curve of the competition of F-NRG-1 and NRG-1, with an EC₅₀ of 4.1*10⁻⁸ M.

FIG. 2C shows the dose-response curve of the competition F-NRG-1 and compound 1. FIG. 2B shows dose-response curve of the competition of F-NRG-1 and compound 6. From the results obtained it can be inferred that the compounds show no competition with NRG-1, instead, they bind allosterically to ERBB4.

AK Assay 96-Well Format

EFO-21 cells were seeded at 10,000 cells/well in 100 μL cell medium in white walled 96-well plates (PerkinElmer, 6005680) and incubated overnight at 37° C. in humidified atmosphere of 5% CO2 in air. Next, cells were put on serum starved (SS) medium (0,1% FBS) overnight. Overnight cell cultures were then stimulated with PBS, compound (32 μM, Enamine) and/or NRG-1 (0.1 μM, Peprotech, 100-03) for 24 hours at 37° C. in humidified atmosphere of 5% CO2 in air. All wells had a final concentration of 0,9% DMSO and compounds were always 10 minutes pre-incubated before addition of NRG-1. Next, 100 μL Toxilight AK detection reagent was added to each well and incubated at room temperature for 5 minutes. Luminescence was measured using the Luminoskan Ascent (Thermofisher).

Cell Proliferation

Cell proliferation was determined by the WST-1 Cell Proliferation Assay Kit (Roche Applied Science). 5,000 EFO-21 cells were seeded into each well of a 96-well plate, allowed to attach for 6 hours and then treated with PBS, compound (32 μM, Enamine) and/or NRG-1 (0.1 μM, Peprotech, 100-03) for 24h. All wells had a final concentration of 0,9% DMSO and compounds were always 10 minutes pre-incubated before addition of NRG-1. Cells were incubated with WST-1 reagent for 4h before harvesting at the indicated time points. Both 450 nm and 650 nm (as a reference) absorbance were measured using Epoch platereader (Biotek).

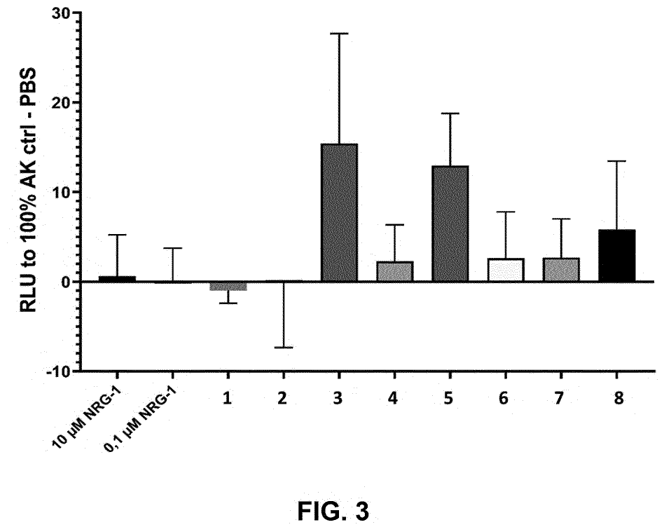

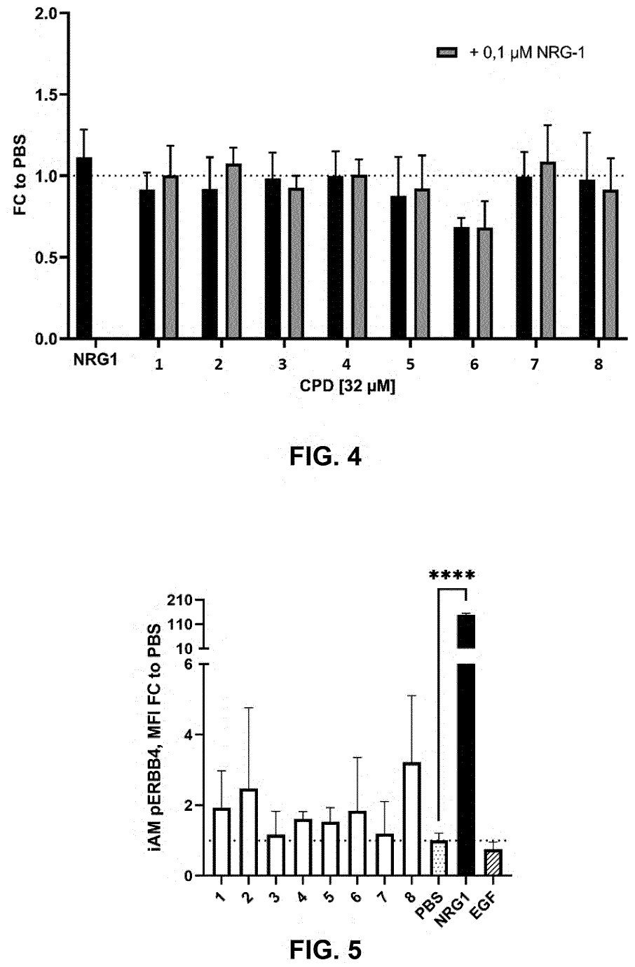

FIG. 3 shows the toxicity of the compounds measured at the highest concentration tested (32 μM). Only compounds 3 and 5 show limited toxicity to EFO-21 cells. ERBB4 receptors are present in EFO-21 cells. FIG. 4 shows the effect of the compounds alone, or in combination with NRG-1 in the cell proliferation of EFO-21 cancer cells. It can be observed that the NRG-1 does not induce cell proliferation of the EFO-21 cells; the compounds of the invention, either combined or not with NRG-1 also do not induce cancer cell proliferation in EFO-21 cells.

Downstream Pathway Activation

Sample Preparation

EFO-21 cells were seeded at 0.4·10⁶ cells/well in 500 μL cell medium in 12-well plates and incubated overnight at 37° C. in humidified atmosphere of 5% CO2 in air. Next, cells were put on SS medium (0,1% FBS) for 8 hours and then stimulated with compound (32 μM, Enamine), NRG-1 (0.1 μM, Peprotech, 100-03), Epidermal Growth factor (EGF) (100 ng/mL, Peprotech, AF-100 15) or PBS for 10 minutes. All wells had a final concentration of 0,9% DMSO. After stimulation, supernatant was discarded and cells rinsed with ice cold TBS 1×. Next, cells were lysed using 150 μL/well 1× milliplex lysis buffer (added in kit) supplemented with protease inhibitors (cOmplete mini [EDTA-free], Roche). Adherent cells were scraped off the well with a cell scraper and then transferred to centrifuge tubes and gently rocked for 10-15 minutes at 4° C. Next particulate matter was removed by using EMD Millipore filters (Merck Millipore, UFC30DV00). Lysates were stored at −80° C. for several weeks before use.

Luminex Total Akt/Phospho Akt Assay

Assays were run according to manufacturer's instructions (48-618MAG, Millipore). Briefly, samples (350 μg/mL total protein concentration) were mixed with antibody-linked magnetic beads on a 96-well plate and incubated overnight at 4° C. with shaking (16-20 h, 750 rpm). Assay buffer was used for the blank wells and the incorporated cell lysates of the kit were used as unstimulated and stimulated control samples. Plates were washed twice using assay buffer and a hand-held magnetic block (Millipore, 40-285). Following a 1h incubation at room temperature with biotinylated detection antibody, plates were washed as above and afterwards streptavidin-PE was added for 15 min with shaking. Next, amplification buffer was added for 15 min with shaking. Plates were washed as above, and 150 μL assay buffer was added to wells. Data were acquired on a validated and calibrated Luminex® 200™ and analysed with xPONENT® 3.1 software with a detection target of 50 beads per region, recommended gate setting of 8,000-15,000.

ERBB4 Phosphorylation

Cells

Immortalized rat atrial cardiomyocytes (iAM, Lab of experimental Cardiology, Leiden University Medical Center, Netherlands) were cultured in Advanced Dulbecco's modified Eagle's medium F-12 (Advanced DMEM/F-12, ThermoFisher Scientific, 12634010) supplemented with 2% (v/v) heat-inactivated fetal bovine serum (FBS, ThermoFisher Scientific, 10082147), 1% (v/v) penicillin/streptomycine, 1% (v/v) GlutaMAX (ThermoFisher Scientific, 35050061) and doxycycline 100 ng/mL (Tocris, 4090), and maintained at 37° C. in humidified atmosphere of 5% CO2 in air. To differentiate, iAM cells were maintained in Advanced DMEM/F-12 medium supplemented with 2% (v/v) FBS and 1% (v/v) GlutaMAX and wells were coated with 0.1 mg/mL fibronectin (R&D systems, 1030-FN-05M).

Sample Preparation

iAM cells were seeded at 1.2×10⁶ cells/mL in 1 mL differentiation medium in 6-well plates and incubated for 9 days (d0=day of seeding) at 37° C. in humidified atmosphere of 5% CO2 in air. Every other day, differentiation medium was refreshed. At d9, cells were put on 500 μL differentiation medium and stimulated with compound (32 μM), NRG1 (0.1 μM, Peprotech, 100-03), Epidermal Growth factor (EGF) (100 ng/mL, Peprotech, AF-100-15) or PBS for 10 minutes. All wells had a final concentration of 0.9% DMSO. After stimulation, supernatant was discarded and cells were rinsed with ice cold TBS 1×. Next, per condition 3 wells needed to be pooled for sufficient amount of protein. Cells were lysed using 150 μL, per condition (pooling 3 wells), of 1× milliplex lysis buffer (added in kit) supplemented with protease inhibitors (cOmplete mini [EDTA-free], Roche). Adherent cells were scraped off the well with a cell scraper and then transferred to centrifuge tubes and gently rocked for 10-15 minutes at 4° C. Next particulate matter was removed by using EMD Millipore filters (Merck Millipore, UFC30DV00). Lysates were stored at −80° C. for several weeks before use.

Assay

Assays were run according to manufacturer's instructions (HPRTKMAG-01K, Millipore). Briefly, samples (350 μg/mL total protein concentration) were mixed with antibody-linked magnetic beads on a 96-well plate and incubated overnight at 4° C. with shaking (16-20 h, 750 rpm). Assay buffer was used for the blank wells and the incorporated cell lysates of the kit were used as unstimulated and stimulated control samples. Plates were washed twice using assay buffer and a hand-held magnetic block (Millipore, 40-285). Following a 1h incubation at room temperature with biotinylated detection antibody, plates were washed as above and afterwards streptavidin-PE was added for 15 min with shaking. Next, amplification buffer was added for 15 min with shaking. Plates were washed as above, and 150 μL assay buffer was added to wells. Data were acquired on a validated and calibrated Luminex® 200™ and analysed with xPONENT® 3.1 software with a detection target of 50 beads per region, recommended gate setting of 8,000-15,000.

FIG. 5 shows that the compounds slightly increase ERBB4 phosphorylation compared to PBS. The mean fluorescence intensity (MFI) is plotted as the fold change to PBS for every compound or control. Data are represented as mean±SD, n=3 in each group, one-way ANOVA, Dunnett's multiple comparisons test. ****P<0.0001 significant difference compared to PBS.

Selectivity

Cells

EFO-21 (DSMZ, ACC235) were cultured in Dulbecco's modified Eagle's medium (DMEM, ThermoFisher Scientific, 11995065) supplemented with 10% (v/v) heat-inactivated fetal bovine serum (FBS, ThermoFisher Scientific, 10082147), 1% penicillin/streptomycin and 1 mL polymyxin B and were maintained at 37° C. in humidified atmosphere of 5% CO2 in air.

Sample Preparation

EFO-21 cells were seeded at 1.6×10⁶ cells/well in 1000 μL cell medium in 6-well plates and incubated overnight at 37° C. in humidified atmosphere of 5% CO2 in air. Next, cells were put on 500 μL serum starved medium (0.1% FBS) per well for 2 hours and then stimulated with compound (32 μM), NRG1 (0.1 μM, Peprotech, 100-03), Epidermal Growth factor (EGF) (100 ng/mL, Peprotech, AF-100-15) or PBS for 10 minutes. All wells had a final concentration of 0.9% DMSO. After stimulation, supernatant was discarded and cells rinsed with ice cold TBS 1×. Next, cells were lysed using 150 μL/well 1× milliplex lysis buffer (added in kit) supplemented with protease inhibitors (cOmplete mini [EDTA-free], Roche). Adherent cells were scraped off the well with a cell scraper and then transferred to centrifuge tubes and gently rocked for 10-15 minutes at 4° C. Next particulate matter was removed by using EMD Millipore filters (Merck Millipore, UFC30DV00). Lysates were stored at −80° C. for several weeks before use.

Assay

Assays were run according to manufacturer's instructions (HPRTKMAG-01K, Millipore). Briefly, samples (350 μg/mL total protein concentration) were mixed with antibody-linked magnetic beads on a 96-well plate and incubated overnight at 4° C. with shaking (16-20 h, 750 rpm). Assay buffer was used for the blank wells and the incorporated cell lysates of the kit were used as unstimulated and stimulated control samples. Plates were washed twice using assay buffer and a hand-held magnetic block (Millipore, 40-285). Following a 1h incubation at room temperature with biotinylated detection antibody, plates were washed as above and afterwards streptavidin-PE was added for 15 min with shaking. Next, amplification buffer was added for 15 min with shaking. Plates were washed as above, and 150 μL assay buffer was added to wells. Data were acquired on a validated and calibrated Luminex® 200™ and analysed with xPONENT® 3.1 software with a detection target of 50 beads per region, recommended gate setting of 8,000-15,000.

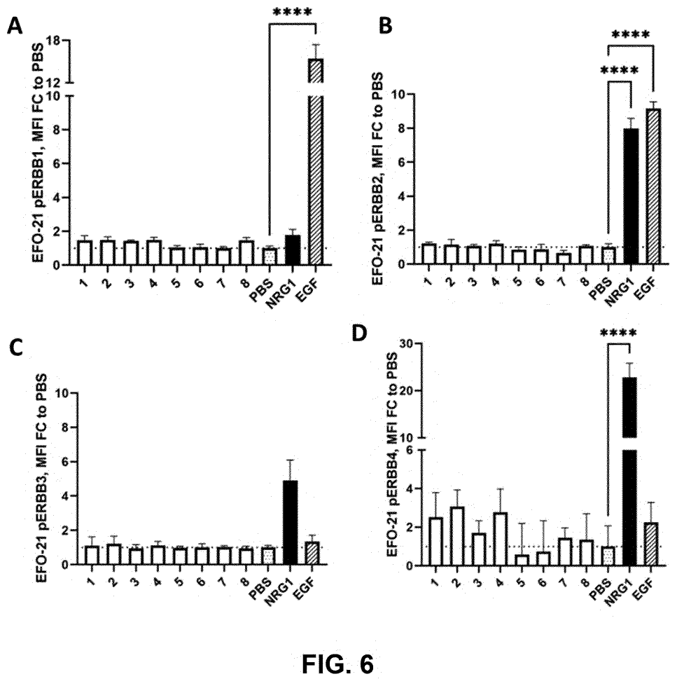

As shown in FIG. 6, the compounds of the invention increase phosphorylation of ERBB4 compared to PBS, but do not increase phosphorylation of ERBB1, ERBB2 or ERBB3 in EFO-21 cells. The mean fluorescence intensity (MFI) is plotted as the fold change (FC) to PBS for every compound or control. Data are represented as mean±SD, n=3 in each group, one-way ANOVA, Dunnett's multiple comparisons test. ****P<0.0001 significant difference compared to PBS.

ERBB2/ERBB3 Heterodimerization Assay

Assay Design

PathHunter® U2OS ERBB2/ERBB3 dimerization cells (DiscoverX, 93-1042C3) have been engineered to co-express one receptor subunit fused to Enzyme Donor (ED), and a second dimer partner fused to Enzyme Acceptor (EA). Cytoplasmic tail may have been deleted from one or both receptors. Binding of an agonist to one receptor subunit induces it to interact with its dimer partner, forcing complementation of the two enzyme fragments. This results in the formation of a functional enzyme (β-galactosidase) that hydrolyzes a substrate to generate a chemiluminescent signal. In this way, the PathHunter Dimerization assay detects ligand induced dimerization of two subunits of a receptor-dimer pair.

Assay

Following 1-12 passages, U2OS ERBB2/ERBB3 dimerization cells were seeded at 10,000 cells/well in 100 μL cell plating reagent (DiscoverX, 93-0563R0A) in white walled 96-well plates (PerkinElmer, 6005680) and incubated for 4h at 37° C. in humidified atmosphere of 5% CO2 in air. Cells were treated with 10 μL of compound (prepared at 11×), 10 μM NRG1, (DiscoverX, 92-1031 or Peprotech, 100-03) or PBS. All wells had a final concentration of 0.9% DMSO. Cells were subsequently incubated for 16 hours at 37° C., 5% CO2. Then 110 μL PathHunter Flash Detection Reagents (93-0247) were added to each well and cells were incubated at room temperature for 1 hour (covered from light). Afterwards luminescence signal was measured using the Luminoskan Ascent (Thermofisher).

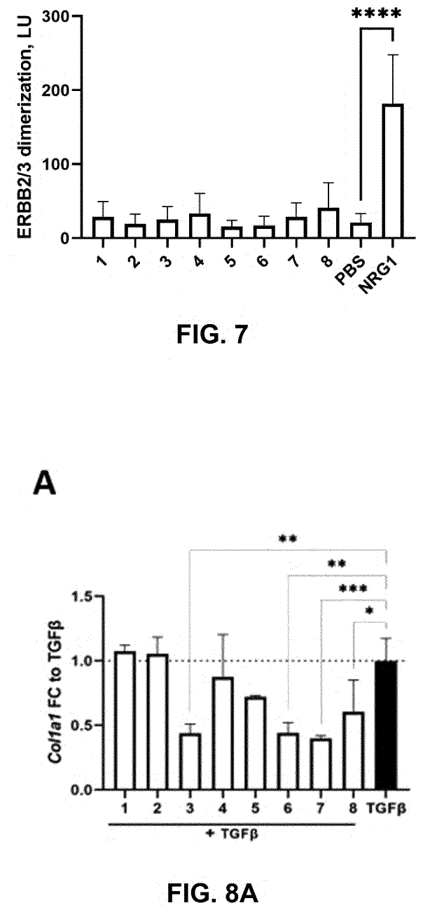

As shown in FIG. 7, the compounds of the present invention do not induce malignant ERBB2/ERBB3 dimerization. Data are represented as mean±SD, n=3 in each group, one-way ANOVA, Dunnett's multiple comparisons test. ****P<0.0001 significant difference compared to PBS. RLU=relative light units.

In Vitro Fibrosis Assay

Cells

Human dermal fibroblasts (HDF, Cell applications, 106-05A) were cultured in Dulbecco's modified Eagle's medium (DMEM, ThermoFisher Scientific, 11995065) supplemented with 10% (v/v) heat-inactivated fetal bovine serum (FBS, ThermoFisher Scientific, 10082147), 1% penicillin/streptomycin and 1 mL polymyxin B and were maintained at 37° C. in humidified atmosphere of 5% CO2 in air. Human atrial cardiac fibroblasts (HCF, Innoprot, P10454) were cultured in fibroblast medium (Innoprot, P60108) supplemented with fetal bovine serum, fibroblast growth supplement and penicillin/streptomycin and were maintained at 37° C. in humidified atmosphere of 5% CO2 in air.

Sample Preparation

Following 1-9 passages, HDF and HCF were seeded at 0.3×10⁶ cells/well in 1 mL cell medium in 12-well plates and incubated overnight at 37° C. in humidified atmosphere of 5% CO2 in air. Next, cells were put on serum starved medium (0.1% FBS) for 8 hours and then stimulated for 24 hours with either PBS, active compound (4-32 μM) or non-active compound (NA1, NA2; 4-32 μM) on top of TGFβ (10 ng/mL, Peprotech, 100-21). All wells had a final concentration of 0.9% DMSO and compounds were 10 minutes pre-incubated before addition of TGFβ. Next, cells were lysed using 100 μL lysis buffer RA1 (of RNA isolation kit below) supplemented with β-mercapto-ethanol (100:1), scraped with a cell scraper and transferred to centrifuge tubes to be stored at −20° C. for a maximum of 2 weeks before RNA isolation.

qRT-PCR

RNA was extracted using Nucleospin RNA XS (Machery Nagel) according to the manufacturer's instructions. For cDNA synthesis, total RNA was added to a solution containing buffer with random hexamers and reverse transcriptase enzyme. The PCR was performed using the following parameters: 10 minutes at 25° C., 30 minutes at 48° C., 5 minutes at 95° C. RT-qPCR on the cDNA samples was performed using Taqman Universal PCR Master Mix (Applied Biosystems) and Taqman primers according to the manufacturer's instructions. Real-time PCR was carried out using QuantStudio 3 Real-time PCR system (Applied Biosystems) and the following parameters: 2 minutes at 95° C. followed by 10 minutes at 95° C., 40 cycles of denaturation at 95° C. for 15 seconds and 1 minute at 60° C. All reactions were run in duplicate and all data were normalized against housekeeping genes GAPDH and PGK1. Expression levels were calculated using the comparative cycle method and expressed as fold change to appropriate (background) control. Following TaqMan primers were used (Thermofisher Scientific): GAPDH (Hs02758991_g1), PGK1 (Hs99999906_m1), COL1A1 (Hs00164004_m1), COL3A1 (Hs00943809_m1).

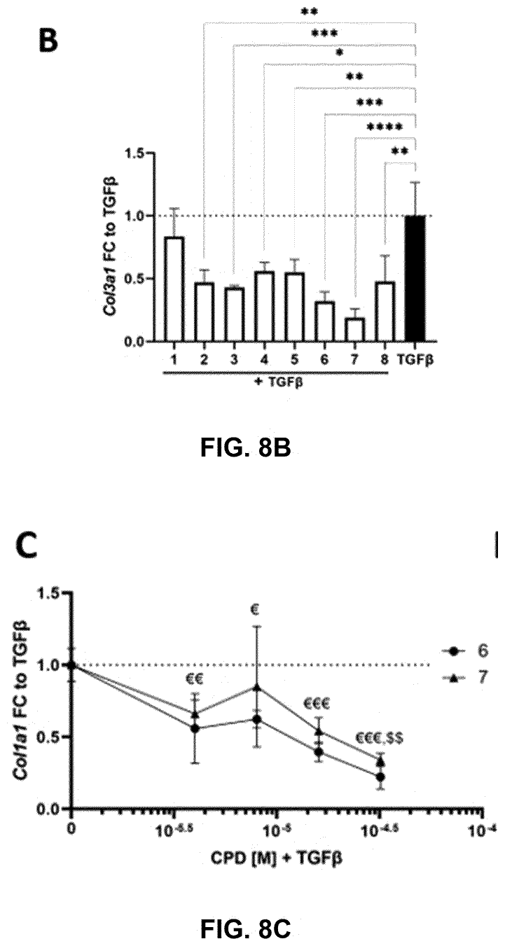

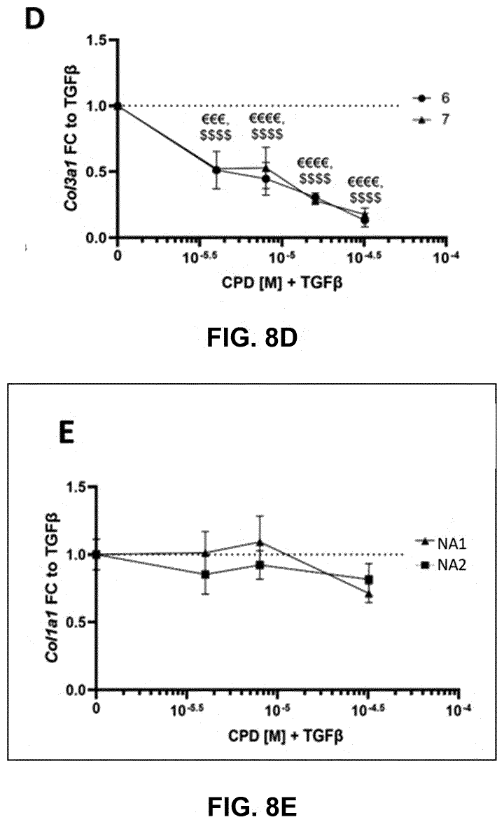

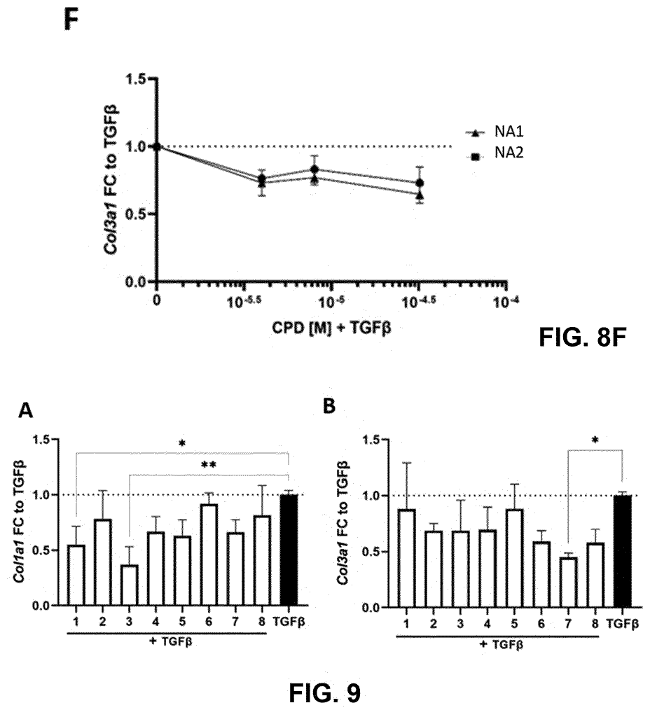

FIG. 8A and FIG. 8B shows that compound 3 and 6 to 8, in comparison with TGFβ control, significantly decrease col1a1 levels and that compound 2 to 8 significantly decrease col3a1 levels after stimulation of HDF with TGFβ. Compound 6 and 7 show significant dose-dependent antifibrotic effects in FIGS. 8C and 8D. FIGS. 8E and 8F, show that non-active compounds (NA1, NA2) have no antifibrotic effect. Data are represented as mean±SD, n=3 in each group, one-way ANOVA, Dunnett's multiple comparisons test. *P<0.05, **P<0.01, ***P<0.001, ****P<0.0001 significant change against TGFβ control. €P<0.05, €€P<0.01, €€€P<0.001, €€€€P<0.0001 significant change of compound 6 against TGFβ control. $$P<0.01, $$$$P<0.0001 significant change of compound 7 against TGFβ control.

FIG. 9 shows that compound 1 and 3, in comparison with TGFβ control, significantly decrease col1a1 levels and that compound 7 significantly decreases col3a1 levels in cultured HCF after stimulation with TGFβ. Data are represented as mean±SD, n=3 in each group, one-way ANOVA, Dunnett's multiple comparisons test. *P<0.05, **P<0.01 significant change against TGFβ control.

In Vitro Pharmacokinetics

Solubility

A dilution series (0.16, 0.31, 0.62, 1.25, 2.5 and 5 mM) was made starting with a compound stock solution of 10 mM in DMSO. Aliquot of 4 μL was taken out of every dilution and added to 196 μL PBS in a 96-well plate. The plate was shaken for 10 seconds, incubated at 37° C. for 2 hours and thereafter absorbance was measured. Samples were analyzed in duplicate.

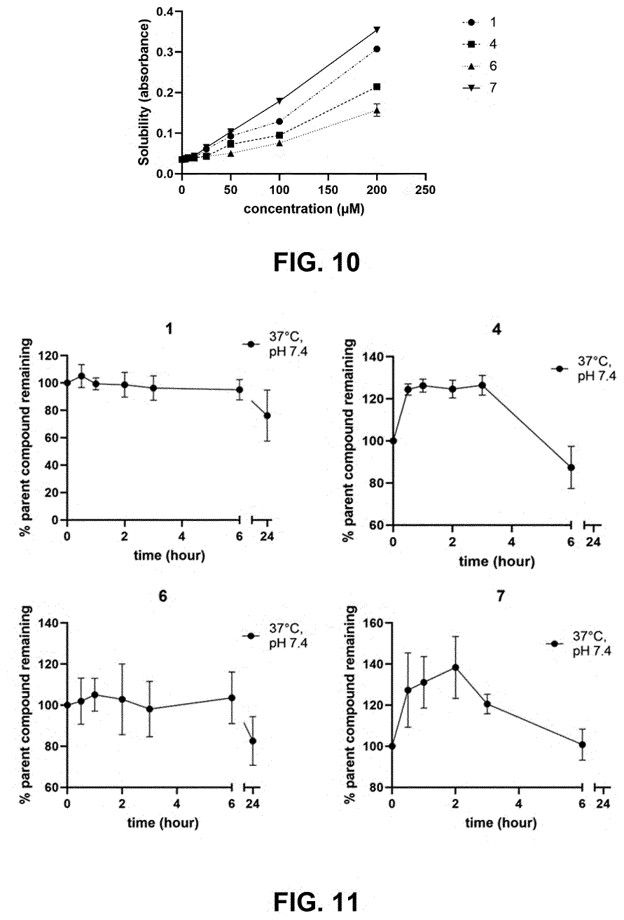

FIG. 10 shows the solubility of compounds of the invention 1, 4, 6 and 7. It was observed that below 25 μM, compounds 1, 4, 6 and 7 are easily soluble. From 50 μM to 200 μM compounds show precipitation.

Log D

Saturated buffers were made by adding 100 mL PBS to 25 mL octanol (Acros) (saturated octanol), and 100 mL octanol to 25 mL PBS (10 mM potassium phosphate, pH 7.4, BD Gentest) (saturated PBS). A 20 μL aliquot of compound solution (10 mM in DMSO) was added to 990 μL saturated PBS. Thereafter 990 μL saturated octanol was added and the solution was shaken for 2 hours at 37° C. After shaking, the solution was kept at room temperature for 10 minutes to initiate separation. A 4 μL aliquot of the octanol (upper) layer was added to 996 μL methanol (CHROMASOLV, 34860).The concentration of compound in the PBS phase and the diluted octanol-aliquot was analyzed with LC-MS/MS (Waters Acquity H-class UPLC system with ion trap mass spectrometer, Bruker Daltonics Esquire 3000 plus, and Agilent 1100 Series LC system). Samples were analyzed in triplicate and plotted against a standard curve (200 μM compound solution in DMSO was further diluted to a series of 50-1600 nM in methanol). Thereafter, Log D was calculated. Table 4 shows the results of the Log D calculation.

TABLE 4

| Compound | LogD |
|---|---|
| 1 | 1.675 ± 0.004 |
| 4 | 2.2 ± 0.1 |
| 6 | 2.0 ± 0.3 |
| 7 | 1.6 ± 0.1 |

Compounds of the invention 1, 4, 6 and 7 had a Log D between 0 and 3, indicating a good preclinical drug.

### Chemical Stability

A 10 μL aliquot of compound solution (10 mM in DMSO) was added to 990 μL DMSO (100 μM) and further diluted with DMSO to a stock solution of 500 nM. The mixture was incubated at 37° C. Aliquots of 100 μL were taken at various time points (0, 0.5, 1, 2, 3, 6 and 24 hrs) and analyzed with LC-MS/MS (Waters Acquity H-class UPLC system with ion trap mass spectrometer, Bruker Daltonics Esquire 3000 plus, and Agilent 1100 Series LC system). Samples were analyzed in triplicate and plotted against a standard curve; compound at 31-500 nM in buffer solution of pH 7.4 (10 mM Potassium Phosphate, BD Gentest).

FIG. 11 shows the chemical stability of the compounds of the invention at physiological pH. Compound 1, 4, 6 and 7 remained chemically stable at 37° C. and pH 7.4 for 24 hours.

Plasma Stability

A 5 μL aliquot of compound solution (10 mM in DMSO) was added to 995 μL of Non Swiss Albino Mouse Plasma (DivBioScience, Breda, Netherlands) or Pooled Normal Human plasma in sodium citrate to obtain a final concentration of 50 μM compound in plasma. The mixture was gently shaken for 6 hours at 37° C. Aliquots of 100 μL were taken at various time points (0, 0.5, 1, 2 and 6 hrs.) and diluted with 400 μL of cold acetonitrile (Sigma-Aldrich, stored at 4° C.). The suspension was centrifuged at 14000 rpm for 5 min. Thereafter, 50 μL of the supernatant was diluted with 950 μL of acetonitrile and analyzed with LC-MS/MS (Waters Acquity H-class UPLC system with ion trap mass spectrometer, Bruker Daltonics Esquire 3000 plus, and Agilent 1100 Series LC system). Samples were analyzed in triplicate and plotted against a standard curve (compound at 3.12-100 μM in plasma and diluted in acetonitrile as described above).

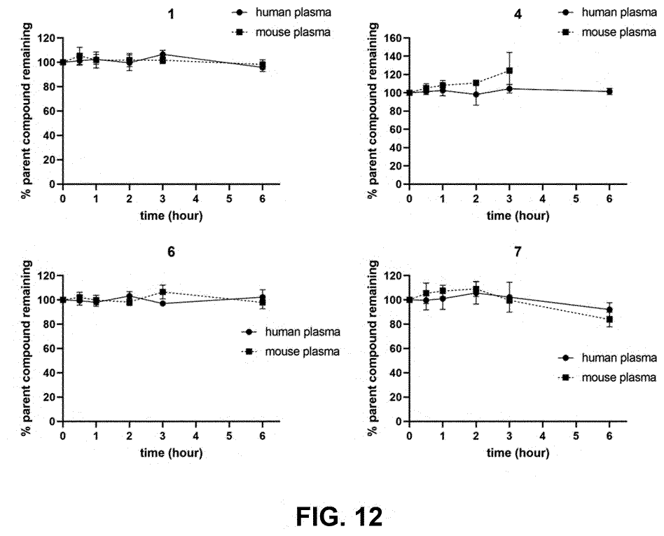

FIG. 12 shows the plasma stability of the compounds of the invention in human and mouse plasma. Compound 1, 4, 6 and 7 were stable in human and mouse plasma over 6 hours.

Metabolic Stability

A mixture of 713 μL milliQ water (Millipore, MA, USA), 200 μL 0.5 M phosphate buffer (pH 7.4, BD Diagnostic Systems, MD, USA), 50 μL NADPH regenerating system solution A (BD Biosciences, CA, USA), 10 μL NADPH regenerating system solution B (BD Biosciences, CA, USA) and 2 μL compound (5 mM in DMSO) was prepared and heated for 5 min at 37° C. Twenty-five μL of liver microsomes (0.5 mg protein/mL, Corning B.V. Life Sciences, Amsterdam, Netherlands) was added to the mixture and 20 μL samples were withdrawn at 0, 0.25, 0.5 and 1, 2, 4, 6 and 24 hours. Eighty μL (4×20 μL) of cold acetonitrile (Sigma-Aldrich, MO, USA) was added to the samples on ice for 10 min. Afterwards, the mixtures were centrifuged at 13,000 rpm for 5 min at 4° C. Seventy-five μL of an acetonitrile/water (10/90) mixture was added to 25 μL supernatant and the obtained samples were analyzed by LC-MS/MS (Waters Acquity H-class UPLC system with ion trap mass spectrometer, Bruker Daltonics Esquire 3000 plus, and Agilent 1100 Series LC system) in triplicate.

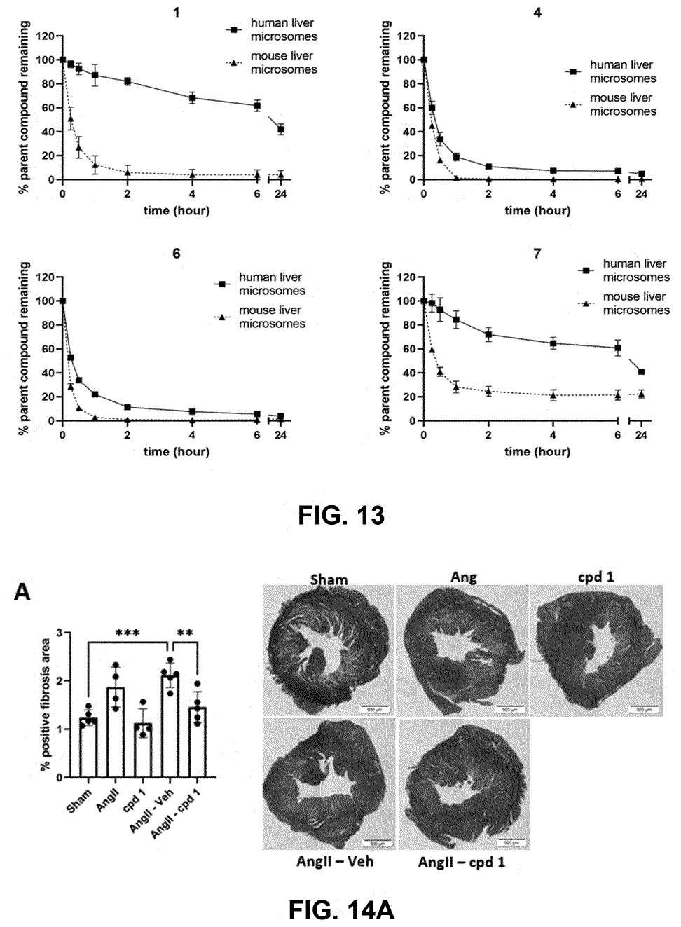

FIG. 13 shows the metabolic stability of the compounds of the invention in human and mouse liver microsomes. Table 5 depicts the half time of compounds in human and mouse liver microsomes

TABLE 5

| Compound | t_{1/2} human (min) | t_{1/2} mouse (min) |
|---|---|---|
| 1 | 228 | 15 |
| 4 | 18 | 12 |
| 6 | 17 | 8 |
| 7 | 173 | 14 |

Compound 1 and 7 were very stable in human liver microsomes, with a half-life (t_{1/2}) above 60 minutes. Compound 4 and 6 were less stable and had a t_{1/2} of 17-18 minutes in human liver microsomes. In mouse liver microsomes, the compounds were less stable than in human liver microsomes. Compound 1 and 7 had a t_{1/2} of 14-15 minutes, compound 4 and 6 a t_{1/2} of 8-12 minutes.

In Vivo Anti-Fibrotic Capacity

Study Approval

All experiments were approved by the ethical committee of the University of Antwerp and conform to the Guide for the Care and Use of Laboratory Animals, 8th edition published by the US National Institutes of Health in 2011, and with the European Communities Council Directive 2010/63/EU for the protection of animals used for experimental purposes.

Mouse Model

Left ventricular (LV) remodeling was induced in 13-week old C₅₇BL/6N male mice by Angiotensin II (AngII, 1000 ng/kg/min, Sigma Aldrich) using a subcutaneous micro-osmotic pump (model 1004, Alzet). Next to AngII, the mice were also given a second s.c. micro-osmotic pump filled with selected hit compound (83 μg/kg/h) or vehicle. Control groups (sham, compound alone, AngII alone) were also implemented. After 4 weeks of treatment, mice were euthanized and hearts were collected.

Histology and Immunostaining

Hearts were fixed and embedded in paraffin. The LV interstitial collagen fraction was determined in Masson trichrome blue-stained areas. Cardiomyocyte outlines were stained with antibodies specific for laminin (NB300-144; Novus Biologicals), and cardiomyocyte cross-sectional area was determined by averaging 20 representative cardiomyocytes per section per animal (5 sections/animal). Data was skewed, therefore log transformation was performed. Endothelial cells were stained with isolectin B4 (Vector Laboratories) and capillary density was determined as the number of capillaries per 100 cardiomyocytes by averaging the number of capillaries per cardiomyocytes on 5 images per animal. Capillaries and cardiomyocytes were counted (by an investigator blinded to the treatment protocol) per section per animal (5 sections/animal). Capillaries were normalized to tissue area to calculate capillary density.

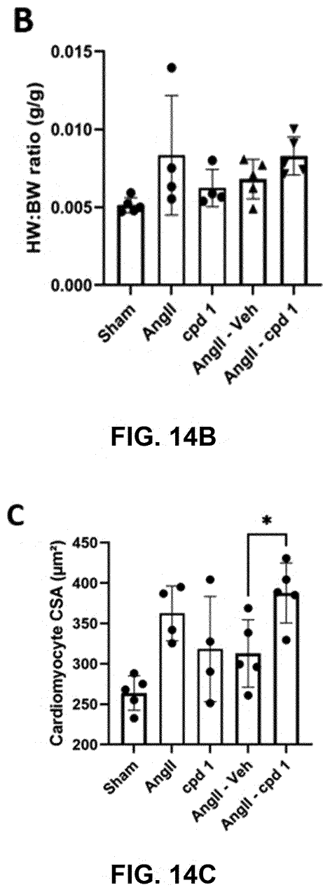

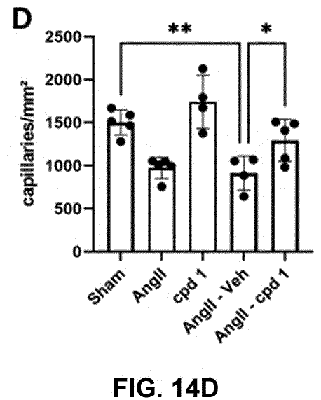

FIG. 14 shows the histological analysis of the hearts excised after 4 weeks of treatment (sham, angiotensin II, compound 1, angiotensin II/vehicle, angiotensin II/compound 1). When compound of the invention 1 was given together with angiotensin II, it significantly attenuated myocardial fibrosis compared to the angiotensin II/vehicle control group. Add-on treatment with compound 1 to angiotensin II had no effect on HW:BW ratio but did increase cardiomyocyte cross sectional area compared to angiotensin II/vehicle. Furthermore, compound of the invention 1 significantly increased capillary density compared to vehicle. Data are represented as mean±SD, n=4-5 in each group, one-way ANOVA, Dunnett's multiple comparisons test.

In Vivo Acute Toxicity

Study Approval

All experiments were approved by the ethical committee of the University of Antwerp and conform to the Guide for the Care and Use of Laboratory Animals, 8th edition published by the US National Institutes of Health in 2011, and with the European Communities Council Directive 2010/63/EU for the protection of animals used for experimental purposes.

Mouse Model and Histological Analysis

An acute toxicity study was performed on C57BL/6N mice using accumulative doses of compound 1 (n=3) or vehicle (n=3, 10% DMSO). On day 1, mice received 0.4 mg/kg cpd 1, day 2 1.2 mg/kg, day 3 4 mg/kg, day 4 4 mg/kg by intraperitoneal injection. At day 4, 1 hour after injection of 4 mg/kg, the mice were sacrificed and the hearts, liver, and kidney excised. The histology of the organs was evaluated by hematoxylin and eosin staining.

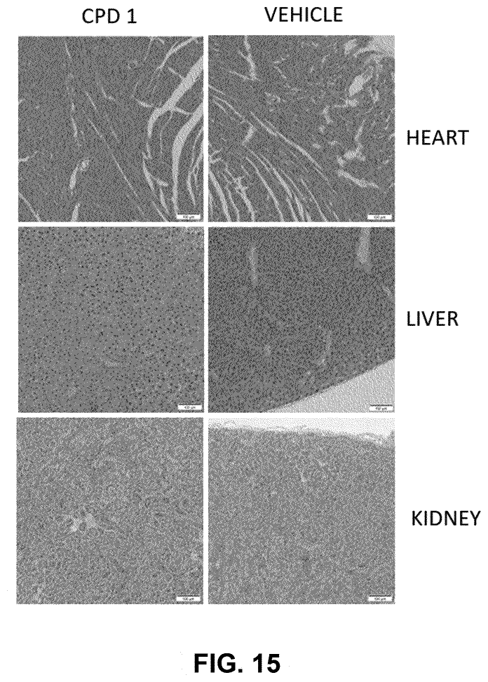

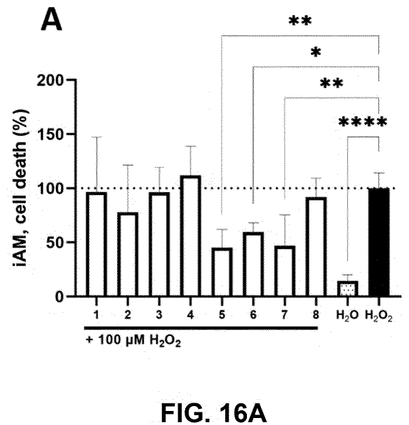

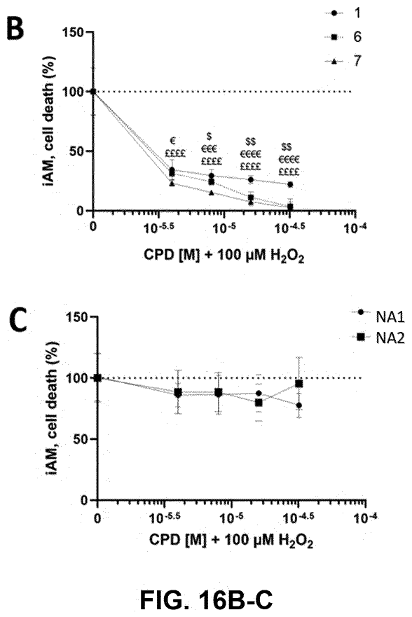

FIG. 15 shows representative images hematoxylin and eosin staining. Acute toxicity was visually evaluated in heart-, liver- and kidney tissue of mice treated with compound of the invention 1 or vehicle. The animals receiving compound 1 exhibited no signs of toxicity. As shown in FIG. 7, this was also confirmed by the histology of the heart, liver and kidney. There was no significant difference in structures of cardiac, liver and renal cells between treated and control group. Because the compounds contain the same pharmacophore, have equivalent molecular weights and are non-toxic in vitro, we assume that the other 5 compounds are also safe for in vivo experiments.

H₂O₂-Induced Cell Death Assay

Cells

Immortalized rat atrial cardiomyocytes (iAM, Lab of experimental Cardiology, Leiden University Medical Center, Netherlands) were cultured in Advanced Dulbecco's modified Eagle's medium F-12 (Advanced DMEM/F-12, ThermoFisher Scientific, 12634010) supplemented with 2% (v/v) heat-inactivated fetal bovine serum (FBS, ThermoFisher Scientific, 10082147), 1% (v/v) penicillin/streptomycine, 1% (v/v) GlutaMAX (ThermoFisher Scientific, 35050061) and doxycycline 100 ng/mL (Tocris, 4090), and maintained at 37° C. in humidified atmosphere of 5% CO2 in air. To differentiate, iAM cells were maintained in Advanced DMEM/F-12 medium supplemented with 2% (v/v) FBS and 1% (v/v) GlutaMAX and wells were coated with 0.1 mg/mL fibronectin (R&D systems, 1030-FN-05M).

Sample Preparation

IAM cells were seeded at 0.4×10⁶ cells/well in 250 μL differentiation medium in 48-well plates and incubated for 9 days (d0=day of seeding) at 37° C. in humidified atmosphere of 5% CO2 in air. Every other day, differentiation medium was refreshed. At d9, cells were put on 200 μL differentiation medium. Next, cells were 10 minutes pretreated with 10 μL of compound (10 μM, or in dose-response at 4/8/16 and 32 μM, prepared at 22×). Control wells were treated with either 10 μL PBS or non-active compound (NA1, NA2; 4/8/16 and 32 μM, prepared at 22×) for 10 minutes. Afterwards 10 μL H₂O₂ (100 μM, Fisher, BP2633-500) was added and cells were incubated for 4 hours at 37° C. in humidified atmosphere of 5% CO2 in air. All wells had a final concentration of 0.9% DMSO, and final volume of 220 μL. Next, 150 μL of every well (48-plate) was transferred to a well of a 96-plate, and 100 μL Toxilight AK detection reagent (ToxiLight, LT07-117) was added to each well and incubated at room temperature for 5 minutes. Luminescence was measured using the Luminoskan Ascent (Thermofisher).

The results are shown in FIG. 16. Data are represented as mean±SD, n=3-4 in each group, one-way ANOVA, Dunnett's multiple comparisons test. *P<0.05, **P<0.01, ****P<0.0001 significant change against 100 μM H₂O₂ control. $P<0.05, $$P<0.01 significant change of compound 1 against H₂O₂ control. €P<0.05 significant change of compound 6 against 100 μM H₂O₂ control. ££££P<0.0001 significant change of compound 7 against H₂O₂ control.

Angiotensin II-Induced Cellular Hypertrophy Assay

Cells

Immortalized rat atrial cardiomyocytes (iAM, Lab of experimental Cardiology, Leiden University Medical Center, Netherlands) were cultured in Advanced Dulbecco's modified Eagle's medium F-12 (Advanced DMEM/F-12, ThermoFisher Scientific, 12634010) supplemented with 2% (v/v) heat-inactivated fetal bovine serum (FBS, ThermoFisher Scientific, 10082147), 1% (v/v) penicillin/streptomycine, 1% (v/v) GlutaMAX (ThermoFisher Scientific, 35050061) and doxycycline 100 ng/mL (Tocris, 4090), and maintained at 37° C. in humidified atmosphere of 5% CO2 in air. To differentiate, iAM cells were maintained in Advanced DMEM/F-12 medium supplemented with 2% (v/v) FBS and 1% (v/v) GlutaMAX and wells were coated with 0.1 mg/mL fibronectin (R&D systems, 1030-FN-05M).

Sample Preparation

IAM cells were seeded at 0.5×10⁵ cells/well in 500 μL differentiation medium in 24-well plates and incubated for 9 days (d0=day of seeding) at 37° C. in humidified atmosphere of 5% CO2 in air. Every other day, differentiation medium was refreshed. At d9, cells were put on 500 μL differentiation medium. Next, cells were 10 minutes pretreated with 25 μL of compound (10 μM, prepared at 22×). Control wells were treated with either 25 μL PBS or non-active compound (NA1, NA2; 10 μM, prepared at 22×) for 10 minutes. Afterwards 25 μL angiotensin II (100 nM, Sigma-Aldrich, A9525) was added and cells were incubated for 24 hours at 37° C. in humidified atmosphere of 5% CO2 in air. All wells had a final concentration of 0.9% DMSO, and final volume of 550 μL.

Histology and Immunostaining

After 24 hour stimulation, cells were fixated with 4% formaldehyde (methanol-free) in PBS for 30 minutes at 4° C. Thereafter, cells were washed in PBS (3×5 min), permeabilized with 0.1% triton X-100 (Acros Organics, AC215682500) in PBS for 10 minutes at room temperature and washed again (PBS, 3×5 min). Next, cells were stained with 1× Alexa Fluor™ 488 Phalloidin (ThermoFisher Scientific, A12379), supplemented with 1% bovine serum albumin (BSA; Sigma Aldrich, A7906) for 1 hour at room temperature. After washing (PBS, 3×5 min), cells were counterstained with 1 μg/mL DAPI (Sigma Aldrich, D9542) for 7 minutes at room temperature. After washing again (3×5 min), the cells were imaged using Celena® S digital imaging system (Logos biosystems). Mean cardiomyocyte cross-sectional area (CSA, μm²) was determined by averaging 5 representative cardiomyocytes per picture per condition (4 digitalized microscopic images/condition).

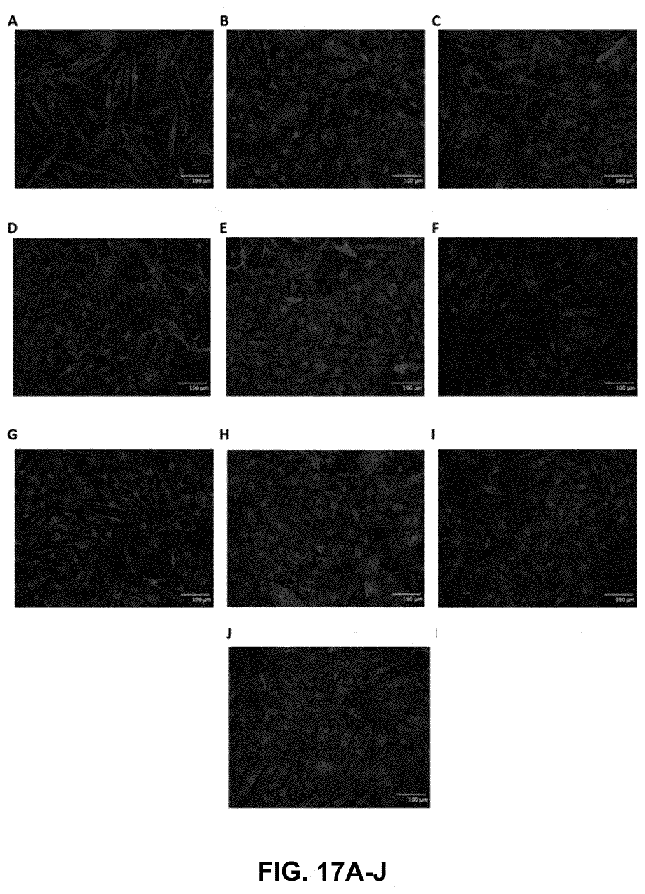

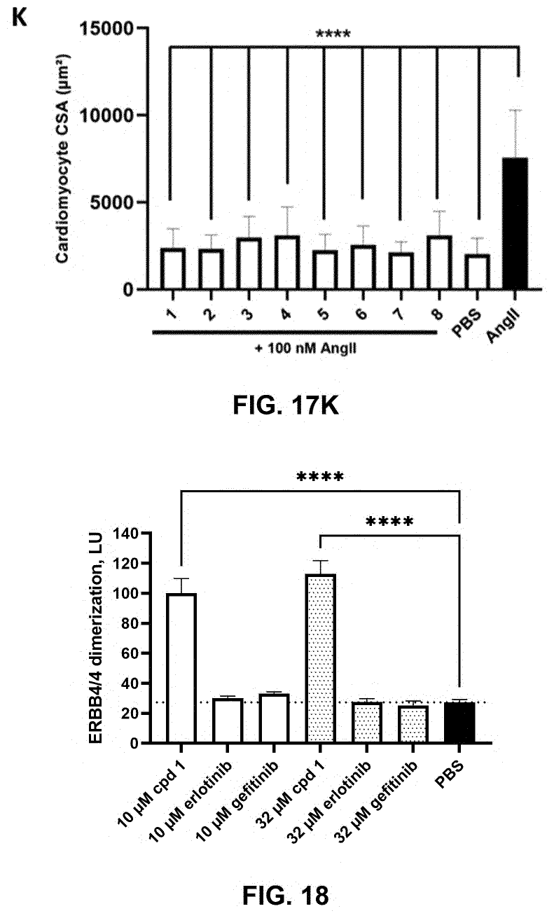

FIG. 17 shows the histological analysis of the angiotensin II-induced cellular hypertrophy assay. All compounds significantly decreased cardiomyocyte CSA in iAM cells after stimulation with AngII, compared to the respective control group. Data are represented as mean±SD, n=3-12 in each group, one-way ANOVA, Dunnett's multiple comparisons test, **** P<0.0001 significant change compared to 100 nM AngII control.

### Comparative Example

Effect of Erlotinib and Gefitinib on ERBB4/4 Homodimerization Assay

PathHunter® Dimerization Assay in 96-Well Format

U2OS ERBB4/ERBB4 dimerization cell line (Eurofins, 93-0961C₃) were seeded at 10,000 cells/well in 100 μL cell plating reagent (Eurofins, 93-0563R0A) in white walled 96-well plates (PerkinElmer, 6005680) and incubated for 24 h at 37° C. in humidified atmosphere of 5% CO2 in air. Cells were treated with 10 μL of 10 μM and 32 μM compound 1 (prepared at 11×, Enamine), erlotinib (Sigma, SML2156)

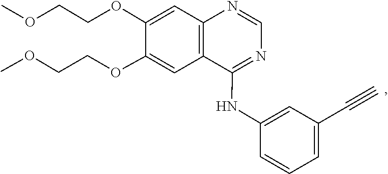 [SMILES: C#Cc1cccc(Nc2ncnc3cc(OCCOC)c(OCCOC)cc23)c1]

gefitinib (Sigma, SML1657)

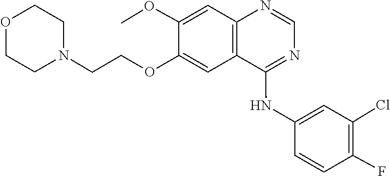 [SMILES: COc1cc2ncnc(Nc3ccc(F)c(Cl)c3)c2cc1OCCN1CCOCC1]

or PBS. All wells had a final concentration of 0.9% DMSO. Cells were subsequently incubated for 6 hours at 37° C., 5% CO2. Then 110 μL PathHunter Flash Detection Reagents (93-0247) were added to each well and cells were incubated at room temperature for 1 hour (covered from light). Afterwards luminescence signal was measured using the Luminoskan Ascent (Thermofisher).

FIG. 18 shows the ERBB4/ERBB4 dimer formation for compound 1, erlotinib and gefitinib in comparison to PBS. Data are represented as mean±SD, N=3 in each group, one-way ANOVA, Dunnett's multiple comparisons test. ****P<0.0001 significant difference compared to PBS. LU=light units.

1. A compound of formula (I) or a stereoisomer, or tautomer thereof, 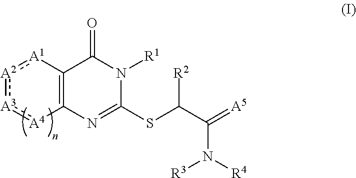
wherein,
each dotted line represents an optional double bond whereby maximally 2 non-adjacent dotted lines can form a double bond;
n is an integer selected from 0, 1, or 2;
A¹ is selected from the group consisting of CR⁵, S, N, NR⁶, and O;
A² is selected from the group consisting of CR⁵, S, N, NR⁶, and O;
A³ is selected from the group consisting of CR⁵, S, N, NR⁶, and O;
A⁴ is selected from the group consisting of CR⁵, S, N, NR⁶, and O;
wherein at most two of A¹ to A⁴ is selected from S, N, NR⁶, and O;
A⁵ is O or NR⁷;
R¹ is selected from the group consisting of alkyl, cycloalkyl, alkenyl, aryl, arylalkyl, heterocyclyl, and heteroaryl; wherein said alkyl, cycloalkyl, alkenyl, aryl, arylalkyl, heterocyclyl, or heteroaryl can be unsubstituted or substituted with one or more Z¹;
R² is selected from the group consisting of hydrogen, alkyl, cycloalkyl, aryl, arylalkyl, heterocyclyl, and heteroaryl; wherein said alkyl, cycloalkyl, aryl, arylalkyl, heterocyclyl, or heteroaryl can be unsubstituted or substituted with one or more Z²;
R³ is selected from the group consisting of hydrogen, cycloalkyl, aryl, alkyl, haloalkyl, haloalkyloxy, arylalkyl heterocyclyl heteroaryl C(O)R⁸, —CO₂R⁹, —C(O)NR⁹R¹⁰—, —S(O)₂R⁹, and —SO₂NR⁹R¹⁰; wherein said cycloalkyl, aryl, alkyl, arylalkyl, heterocyclyl, or heteroaryl can be unsubstituted or substituted with one or more Z³;
or R² and R³ together with the atom to which they are attached form a saturated or unsaturated 5-, 6-, 7-, 8- or 9-membered ring;
R⁴ is selected from the group consisting of hydrogen, cycloalkyl, aryl, alkyl, haloakyl, haloalkyloxy, arylalkyl, heterocyclyl, and heteroaryl; wherein said cycloalkyl, aryl, alkyl, haloakyl, haloalkyloxy, arylalkyl, heterocyclyl, or heteroaryl can be unsubstituted or substituted with one or more Z⁴;
or R³ and R⁴ together with the nitrogen atom to which they are attached form a saturated or unsaturated 5-, 6-, 7-, 8-, 9-, 10- or 11-membered nitrogen-containing monocycle or bicycle;
R⁵ is selected from the group consisting of hydrogen, alkyl, cycloalkyl, halo, aryl, arylalkyl, heterocyclyl, heteroaryl, hydroxyl, —OR⁸, cyano, amino, nitro, haloalkyl, haloalkyloxy, —C(O)R⁸, —NR⁸R⁹, —CO₂R⁹, —C(O)NR⁹R¹⁰, —S(O)₂R⁹, —S(O)₂NR⁹R¹⁰, —NR⁹C(O)R¹⁰, and —NR⁹S(O)₂R¹⁰;
or wherein two R⁵ together with the atom to which they are attached can form a saturated or unsaturated 5-, 6-, 7-, 8-, 9- or 10-membered ring;
wherein said ring, alkyl, cycloalkyl, aryl, arylalkyl, heterocyclyl, or heteroaryl can be unsubstituted or substituted with one or more Z⁵;
R⁶ is selected from the group consisting of hydrogen, alkyl, cycloalkyl, aryl, arylalkyl, heterocyclyl, heteroaryl, and —S(O)₂R⁹; wherein said alkyl, cycloalkyl, aryl, arylalkyl, heterocyclyl, or heteroaryl can be unsubstituted or substituted with one or more Z⁶;
R⁷ is selected from the group consisting of alkyl, cycloalkyl, aryl, arylalkyl, heterocyclyl, and heteroaryl; wherein said alkyl, cycloalkyl, aryl, arylalkyl, heterocyclyl, or heteroaryl can be unsubstituted or substituted with one or more Z⁷;
or R³ and R⁷ together with the atom to which they are attached form an unsaturated 5-, 6-, 7-, 8-, 9-, 10- or 11-membered nitrogen-containing monocycle or bicycle;
each R⁸ is independently selected from the group consisting of, alkyl, aryl, cycloalkyl, arylalkyl, heterocyclyl, heteroaryl;
each R⁹ is independently selected from the group consisting of hydrogen, alkyl, aryl, cycloalkyl, arylalkyl, heterocyclyl, heteroaryl;
each R¹⁰ is independently selected from the group consisting of hydrogen, alkyl, aryl, cycloalkyl, arylalkyl, heterocyclyl, heteroaryl;
each Z¹ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z² is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z³ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z⁴ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z⁵ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z⁶ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z⁷ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
or a solvate, hydrate, pharmaceutically acceptable salt, or prodrug thereof for use in the prevention or treatment of a disease associated with the activation of receptor tyrosine-protein kinase (ERBB4).

2. The compound for use according to claim 1, having structural formula (IA), (IB) or (IC), 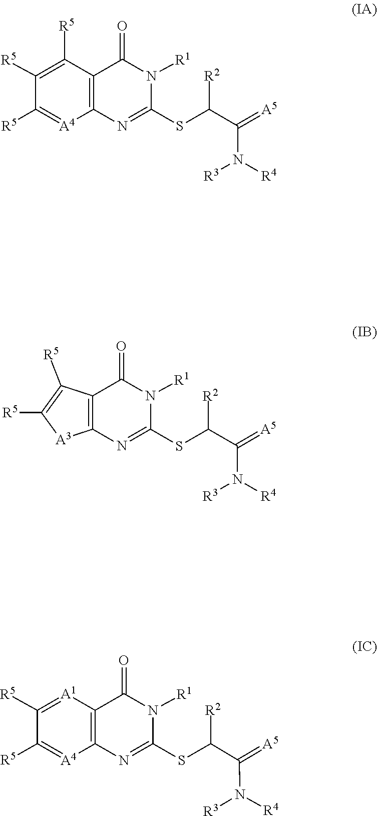
wherein A¹, A⁴, A⁵, R¹, R², R³, R⁴, and R⁵ have the same meaning as that defined in claim 1.

3. The compound for use according to claim 1, having structural formula (IIA), (IIB) (IIC) or (IID), 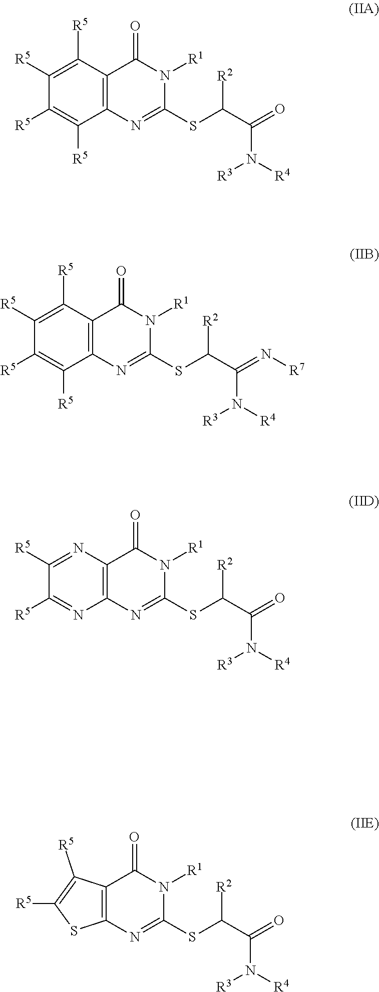
wherein R¹, R², R³, R⁴, and R⁵ have the same meaning as that defined in claim 1.

4. The compound for use according to claim 1, wherein,
A¹ is CR⁵, N, or S;
A² is CR⁵, N, or S;
A³ is CR⁵, N, or S;
A⁴ is CR⁵, N, or S;
R¹ is selected from the group consisting of alkyl, cycloalkyl, alkenyl, aryl, and arylalkyl;
wherein said alkyl, cycloalkyl, alkenyl, aryl, and arylalkyl can be unsubstituted or substituted with one or more Z¹;
R² is selected from the group consisting of hydrogen, alkyl, cycloalkyl, and aryl;
R³ is selected from the group consisting of hydrogen, cycloalkyl, aryl, alkyl, haloalkyl, haloalkyloxy, arylalkyl heterocyclyl heteroaryl, —OR⁸, —CO₂R⁹, —C(O)NR⁹R¹⁰, —S(O)₂R⁹, and —SO₂NR⁹R¹⁰; wherein said cycloalkyl, aryl, alkyl, arylalkyl, heterocyclyl, or heteroaryl can be unsubstituted or substituted with one or more Z³;
or R² and R³ together with the atom to which they are attached form a saturated or unsaturated 5-, 6-, 7-, 8- or 9-membered ring;
R⁴ is selected from the group consisting of hydrogen, cycloalkyl, aryl, alkyl, and haloalkyl;
or R³ and R⁴ together with the nitrogen atom to which they are attached form a saturated or unsaturated 7-, 8-, 9-, or 10-membered nitrogen-containing monocycle or bicycle;
R⁵ is selected from the group consisting of hydrogen, alkyl, cycloalkyl, halo, aryl, arylalkyl, heterocyclyl, heteroaryl, hydroxyl, —OR⁸, cyano, amino, nitro, haloalkyl, haloalkyloxy, —C(O)R⁸, —NR⁸R⁹, —CO₂R⁹, —C(O)NR⁹R¹⁰, —S(O)₂R⁹, —SO₂NR⁹R¹⁰, —NR⁹C(O)R¹⁰, and —NR⁹S(O)₂R¹⁰;
or wherein two R⁵ together with the atom to which they are attached can form a saturated or unsaturated 5-, 6-, or 7-membered ring;
wherein said ring, alkyl, cycloalkyl, aryl, arylalkyl, heterocyclyl, or heteroaryl can be unsubstituted or substituted with one or more Z⁵;
R⁶ is selected from the group consisting of hydrogen, alkyl, and cycloalkyl;
R⁷ is selected from the group consisting of alkyl, cycloalkyl, aryl, arylalkyl, heterocyclyl, and heteroaryl;
or R³ and R⁷ together with the atom to which they are attached form an unsaturated 6-, 7-, 8-, 9-, or 10-membered nitrogen-containing monocycle or bicycle.

5. The compound for use according to claim 1, wherein,
each Z¹ is independently selected from the group consisting of halo, C₁₋₆alkyl, haloC₁₋₄alkyl, haloC₁₋₄alkyloxy, C₃₋₁₂cycloalkyl, C₃₋₁₂cycloalkenyl, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy; C₆₋₁₂aryloxy, heterocyclyloxy, heteroaryloxy, cyano, amino, mono-C₁₋₄alkylamino, mono-C₃₋₁₂cycloalkylamino, mono-C₆₋₁₂arylamino, hydroxycarbonyl, C₁₋₄alkyloxycarbonyl, C₃₋₁₂cycloalkyloxycarbonyl, C₆₋₁₂aryloxycarbonyl, aminocarbonyl, mono-C₁₋₄alkylaminocarbonyl, mono-C₃₋₁₂cycloalkylaminocarbonyl, C₁₋₄alkylcarbonyl, C₃₋₁₂cycloalkylcarbonyl, C₆₋₁₂arylcarbonyl, —S(O)H, C₁₋₄alkylsulfinyl, —S(O)₂H, C₁₋₄alkylsulfonyl, —SO₂NH₂, mono-C₁₋₄alkylaminosulfonyl, nitro;
each Z² is independently selected from the group consisting of halo, C₁₋₆alkyl, haloC₁₋₄alkyl, haloC₁₋₄alkyloxy, C₃₋₁₂cycloalkyl, C₃₋₁₂cycloalkenyl, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, mono-C₁₋₄alkylamino, mono-C₃₋₁₂cycloalkylamino, mono-C₆₋₁₂arylamino, hydroxycarbonyl, C₁₋₄acyloxycarbonyl, C₃₋₁₂cycloakyloxycarbonyl, C₆₋₁₂aryloxycarbonyl, aminocarbonyl, mono-C₁₋₄alkylaminocarbonyl, mono-C₃₋₁₂cycloalkylaminocarbonyl, C₁₋₄alkylcarbonyl, C₃₋₁₂cycloalkylcarbonyl, C₆₋₁₂arylcarbonyl, —S(O)H, C₁₋₄alkylsulfonyl, —S(O)₂H, C₁₋₄alkylsulfonyl, —SO₂NH₂, mono-C₁₋₄alkylaminosulfonyl, nitro;
each Z³ is independently selected from the group consisting of halo, C₁₋₆alkyl, haloC₁₋₄alkyl, haloC₁₋₄alkyloxy, C₃₋₁₂cycloalkyl, C₃₋₁₂cycloalkenyl, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, mono-C₁₋₄alkylamino, mono-C₃₋₁₂cycloalkylamino, mono-C₆₋₁₂arylamino, hydroxycarbonyl, C₁₋₄alkyloxycarbonyl, C₃₋₁₂cycloakyloxycarbonyl, C₆₋₁₂aryloxycarbonyl, aminocarbonyl, mono-C₁₋₄alkylaminocarbonyl, mono-C₃₋₁₂cycloalkylaminocarbonyl, C₁₋₄alkylcarbonyl, C₃₋₁₂cycloalkylcarbonyl, C₆₋₁₂arylcarbonyl, —S(O)H, C₁₋₄alkylsulfinyl, —S(O)₂H, C₁₋₄alkylsulfonyl, —SO₂NH₂, mono-C₁₋₄alkylaminosulfonyl, nitro;
each Z⁴ is independently selected from the group consisting of halo, C₁₋₆alkyl haloC₁₋₄alkyl, haloC₁₋₄alkyloxy, C₃₋₁₂cycloalkyl, C₃₋₁₂cycloalkenyl, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, mono-C₁₋₄alkylamino, mono-C₃₋₁₂cycloalkylamino, mono-C₆₋₁₂arylamino, hydroxycarbonyl, C₁₋₄acyloxycarbonyl, C₃₋₁₂cycloakyloxycarbonyl, C₆₋₁₂aryloxycarbonyl, aminocarbonyl, mono-C₁₋₄alkylaminocarbonyl, mono-C₃₋₁₂cycloalkylaminocarbonyl, C₁₋₄alkylcarbonyl, C₃₋₁₂cycloalkylcarbonyl, C₆₋₁₂arylcarbonyl, —S(O)H, C₁₋₄alkylsulfinyl, —S(O)₂H, C₁₋₄alkylsulfonyl, —SO₂NH₂, mono-C₁₋₄alkylaminosulfonyl, nitro;
each Z⁵ is independently selected from the group consisting of halo, C₁₋₆alkyl, haloC₁₋₄alkyl, haloC₁₋₄alkyloxy, C₃₋₁₂cycloalkyl, C₃₋₁₂cycloalkenyl, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, mono-C₁₋₄alkylamino, mono-C₃₋₁₂cycloalkylamino, mono-C₆₋₁₂arylamino, hydroxycarbonyl, C₁₋₄alkyloxycarbonyl, C₃₋₁₂cycloakyloxycarbonyl, C₆₋₁₂aryloxycarbonyl, aminocarbonyl, mono-C₁₋₄alkylaminocarbonyl, mono-C₃₋₁₂ cycloalkylaminocarbonyl, C₁₋₄alkylcarbonyl, C₃₋₁₂cycloalkylcarbonyl, C₆₋₁₂arylcarbonyl, —S(O)H, C₁₋₄alkylsulfinyl, —S(O)₂H, C₁₋₄alkylsulfonyl, —SO₂NH₂, mono-C₁₋₄alkylaminosulfonyl, nitro;
each Z⁶ is independently selected from the group consisting of halo, C₁₋₆alkyl, haloC₁₋₄alkyl, haloC₁₋₄alkyloxy, C₃₋₁₂cycloalkyl, C₃₋₁₂cycloalkenyl, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, mono-C₁₋₄alkylamino, mono-C₃₋₁₂cycloalkylamino, mono-C₆₋₁₂arylamino, hydroxycarbonyl, C₁₋₄alkyloxycarbonyl, C₃₋₁₂cycloalkyloxycarbonyl, C₆₋₁₂aryloxycarbonyl, aminocarbonyl, mono-C₁₋₄alkylaminocarbonyl, mono-C₃₋₁₂cycloalkylaminocarbonyl, C₁₋₄alkylcarbonyl, C₃₋₁₂cycloalkylcarbonyl, C₆₋₁₂arylcarbonyl, —S(O)H, C₁₋₄alkylsulfinyl, —S(O)₂H, C₁₋₄alkylsulfonyl, —SO₂NH₂, mono-C₁₋₄alkylaminosulfonyl, nitro;
each Z⁷ is independently selected from the group consisting of halo, C₁₋₆alkyl, haloC₁₋₄alkyl, haloC₁₋₄alkyloxy, C₃₋₁₂cycloalkyl, C₃₋₁₂cycloalkenyl, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, mono-C₁₋₄alkylamino, mono-C₃₋₁₂cycloalkylamino, mono-C₆₋₁₂arylamino, hydroxycarbonyl, C₁₋₄alkyloxycarbonyl, C₃₋₁₂cycloalkyloxycarbonyl, C₆₋₁₂alkyloxycarbonyl, aminocarbonyl, mono-C₁₋₄alkylaminocarbonyl, mono-C₃₋₁₂cycloalkylaminocarbonyl, C₁₋₄alkylcarbonyl, C₃₋₁₂cycloalkylcarbonyl, C₆₋₁₂arylcarbonyl, —S(O)H, C₁₋₄alkylsulfinyl, —S(O)₂H, C₁₋₄alkylsulfonyl, —SO₂NH₂, mono-C₁₋₄alkylaminosulfonyl, nitro.

6. The compound for use according to claim 1, wherein,
R¹ is selected from the group consisting of C₁₋₆alkyl, C₃₋₁₂cycloalkyl, C₂₋₆alkenyl, C₆₋₁₂aryl, C₆₋₁₂arylC₁₋₆alkyl and heteroaryl; wherein said groups can be unsubstituted or substituted with one, two or three Z¹;
R² is selected from the group consisting of hydrogen, C₁₋₆alkyl, C₃₋₁₂cycloalkyl and C₆₋₁₂aryl;
R³ is selected from the group consisting of hydrogen, C₃₋₁₂cycloalkyl, C₆₋₁₂aryl, C₁₋₆alkyl, haloC₁₋₄alkyl, haloC₁₋₄alkyloxy, C₆₋₁₂arylC₁₋₆alkyl, heterocyclyl, heteroaryl, C₁₋₄alkylcarbonyl, C₃₋₁₂cycloalkylcarbonyl, C₆₋₁₂arylcarbonyl, C₁₋₄alkyloxycarbonyl, C₃₋₁₂cycloalkyloxycarbonyl, C₆₋₁₂aryloxycarbonyl, aminocarbonyl, mono-C₁₋₄alkylaminocarbonyl, and mono-C₃₋₁₂cycloalkylaminocarbonyl; wherein said groups can be unsubstituted or substituted with one, two or three Z³;
or R² and R³ together with the atom to which they are attached form a saturated unsaturated 6-, 7-, or 8-membered ring;
R⁴ is selected from the group consisting of hydrogen, C₃₋₁₀cycloalkyl, C₆₋₁₂aryl, and haloC₁₋₄alkyl;
or R³ and R⁴ together with the nitrogen atom to which they are attached form a saturated 7-, 8-, 9- or 10-membered nitrogen-containing monocycle or bicycle;
R⁵ is selected from the group consisting of hydrogen, C₁₋₆alkyl, C₃₋₁₂cycloalkyl, halo, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, nitro, haloC₁₋₄alkyl, haloC₁₋₄ alkyloxy, C₁₋₄alkylcarbonyl, C₃₋₁₂cycloalkylcarbonyl, C₆₋₁₂arylcarbonyl, mono-C₁₋₄alkylamino, mono-C₃₋₁₂cycloalkylamino, mono-C₆₋₁₂arylamino, hydroxycarbonyl, C₁₋₄alkyloxycarbonyl, C₃₋₁₂cycloalkyloxycarbonyl, C₆₋₁₂aryloxycarbonyl, aminocarbonyl, mono-C₁₋₄alkylaminocarbonyl, mono-C₃₋₁₂cycloalkylaminocarbonyl, S(O)₂H, C₁₋₄akylsulfonyl, —SO₂NH₂, mono-C₁₋₄alkylaminosulfonyl, C₁₋₄alkylcarbonylamino, and C₁₋₄alkylsulfonylamino;
or wherein two R⁵ together with the atom to which they are attached can form a saturated or unsaturated 5-, 6-, or 7-membered ring;
wherein said ring, C₁₋₆alkyl, C₃₋₁₂cycloalkyl, C₆₋₁₂aryl, C₆₋₁₂arylC₁₋₆alkyl, heterocyclyl, or heteroaryl can be unsubstituted or substituted with one, two or three Z⁵;
R⁶ is selected from the group consisting of hydrogen, C₁₋₆alkyl and C₃₋₁₂cycloalkyl;
R⁷ is selected from the group consisting of C₁₋₆alkyl, C₃₋₁₂cycloalkyl, C₆₋₁₂aryl, C₆₋₁₂arylalkyl, heterocyclyl, and heteroaryl;
or R³ and R⁷ together with the atom to which they are attached form an unsaturated 7-, 8-, 9-, or 10-membered nitrogen-containing monocycle or bicycle.

7. The compound for use according to claim 1, wherein,
R¹ is selected from the group consisting of C₁₋₄alkyl, C₃₋₁₀cycloalkyl, C₂₋₄alkenyl, C₆₋₁₂aryl, C₆₋₁₂arylC₁₋₄alkyl and heteroaryl;
R² is selected from the group consisting of hydrogen, C₁₋₄alkyl, and C₆₋₁₂aryl;
R³ is selected from the group consisting of hydrogen, C₃₋₁₀cycloalkyl, C₆₋₁₂aryl, C₁₋₄alkyl, haloC₁₋₄alkyl, haloC₁₋₄alkyloxy, C₆₋₁₂arylC₁₋₄alkyl, heterocyclyl, heteroaryl, C₁₋₄alkylcarbonyl, C₁₋₄alkyloxycarbonyl, aminocarbonyl, mono-C₁₋₄allylaminocarbonyl, and mono-C₃₋₁₂cycloalkylaminocarbonyl; wherein said groups can be unsubstituted or substituted with one, two or three Z³;
or R² and R³ together with the carbon atom to which they are attached form a saturated 6-, 7-, or 8-membered ring;
R⁴ is selected from the group consisting of hydrogen, C₃₋₈cycloalkyl, C₆₋₁₂aryl, and haloC₁₋₄alkyl;
or R³ and R⁴ together with the nitrogen atom to which they are attached form a saturated 7-, 8-, 9-membered nitrogen-containing monocycle or bicycle;
R⁵ is selected from the group consisting of hydrogen, C₁₋₄alkyl, C₃₋₁₀cycloalkyl, halo, C₆₋₁₂aryl, C₆₋₁₂arylC₁₋₄alkyl, heterocyclyl, heteroaryl, hydroxyl, C₁₋₆alkyloxy, C₃₋₁₀cycloalkyloxy, C₆₋₁₂aryloxy, cyano, amino, nitro, haloC₁₋₄alkyl, haloC₁₋₄alkyloxy, mono-C₁₋₄alkylamino, hydroxycarbonyl, C₁₋₄alkyloxycarbonyl, —CONH₂, mono-C₁₋₄alkylaminocarbonyl, S(O)₂H, C₁₋₄alkylsulfonyl, and —SO₂NH₂;
or wherein two R⁵ together with the atom to which they are attached can form a saturated or unsaturated 5-, 6-, or 7-membered ring;
wherein said ring, C₁₋₄alkyl, C₃₋₈cycloalkyl, C₆₋₁₂aryl, C₆₋₁₂arylC₁₋₄alkyl, heterocyclyl, or heteroaryl can be unsubstituted or substituted with one, two or three Z⁵;
R⁶ is selected from the group consisting of hydrogen, C₁₋₆alkyl and C₃₋₁₀cycloalkyl;
R⁷ is selected from the group consisting of C₁₋₆alkyl, C₃₋₁₀cycloalkyl, C₆₋₁₂aryl, C₆₋₁₂arylalkyl, heterocyclyl, and heteroaryl;
or R³ and R⁷ together with the carbon atom to which they are attached form an unsaturated 7-, 8-, 9-membered nitrogen-containing monocycle or bicycle.

8. The compound according to claim 1, selected from the group consisting of:

| Compound | Structure |
|---|---|
| 1 | 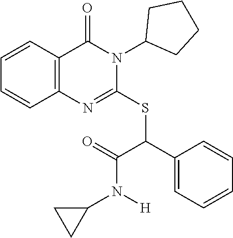 [SMILES: O=C(NC1CC1)C(Sc1nc2ccccc2c(=O)n1C1CCCC1)c1ccccc1] |
| 2 | 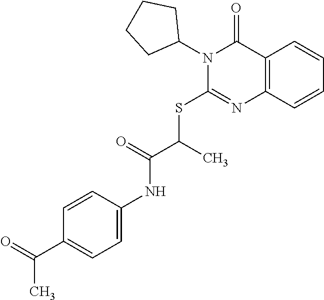 [SMILES: CC(=O)c1ccc(NC(=O)C(C)Sc2nc3ccccc3c(=O)n2C2CCCC2)cc1] |
| 3 | 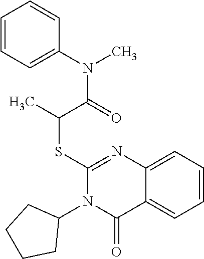 [SMILES: CC(Sc1nc2ccccc2c(=O)n1C1CCCC1)C(=O)N(C)c1ccccc1] |
| 4 | 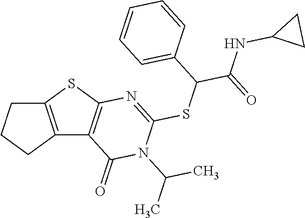 [SMILES: CC(C)n1c(SC(C(=O)NC2CC2)c2ccccc2)nc2sc3c(c2c1=O)CCC3] |
| 5 | 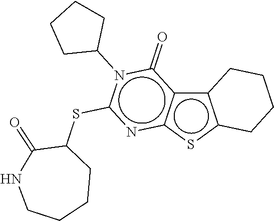 |
| 6 | 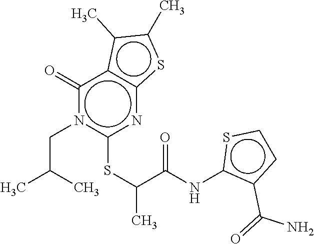 |
| 7 | 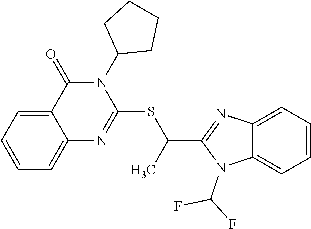 [SMILES: CC(Sc1nc2ccccc2c(=O)n1C1CCCC1)c1nc2ccccc2n1C(F)F] |
| 8 | 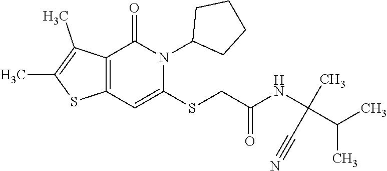 [SMILES: Cc1sc2cc(SCC(=O)NC(C)(C#N)C(C)C)n(C3CCCC3)c(=O)c2c1C] |

9. The compound for use according to claim 1, wherein the disease associated with the activation of receptor tryosine-protein kinase (ERBB-4) is selected from the group consisting of heart failure, metabolic disorders, inflammatory disorders, fibrotic disorders, and cancer.

10. The compound for use according to claim 1, wherein the disease associated with the activation of receptor tyrosine-protein kinase (ERBB) is selected from the group consisting of chronic heart failure, (chronic) diabetic nephropathy, dermal-, pulmonary- and myocardial fibrosis.

11. A compound of formula (I) or a stereoisomer, or tautomer thereof, 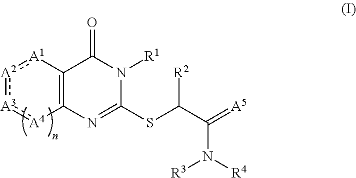
wherein,
each dotted line represents an optional double bond whereby maximally 2 non-adjacent dotted lines can form a double bond;
n is an integer selected from 0, 1, or 2;
A¹ is selected from the group consisting of CR⁵, S, N, NR⁶, and O;
A² is selected from the group consisting of CR⁵, S, N, NR⁶, and O;
A³ is selected from the group consisting of CR⁵, S, N, NR⁶, and O;
A⁴ is selected from the group consisting of CR⁵, S, N, NR⁶, and O;
wherein at most two of A¹ to A⁴ is selected from S, N, NR⁶, and O;
A⁵ is O or NR⁷;
R¹ is selected from the group consisting of alkyl, cycloalkyl, alkenyl, aryl, arylalkyl, heterocyclyl, and heteroaryl; wherein said alkyl, cycloalkyl, alkenyl, aryl, arylalkyl, heterocyclyl, or heteroaryl can be unsubstituted or substituted with one or more Z¹;
R² is selected from the group consisting of hydrogen, alkyl, cycloalkyl, aryl, arylalkyl, heterocyclyl, and heteroaryl; wherein said alkyl, cycloalkyl, aryl, arylalkyl, heterocyclyl, or heteroaryl can be unsubstituted or substituted with one or more Z²;
R³ is selected from the group consisting of hydrogen, cycloalkyl, aryl, alkyl, haloalkyl, haloalkyloxy, arylalkyl heterocyclyl heteroaryl —C(O)R⁸, —CO₂R⁹—C(O)NR⁹R¹⁰, —S(O)₂R⁹, and —SO₂NR⁹R¹⁰; wherein said cycloalkyl, aryl, alkyl, arylalkyl, heterocyclyl, or heteroaryl can be unsubstituted or substituted with one or more Z³;
or R² and R³ together with the atom to which they are attached form a saturated or unsaturated 5-, 6-, 7-, 8- or 9-membered ring;
R⁴ is selected from the group consisting of hydrogen, cycloalkyl, aryl, alkyl, haloakyl, haloalkyloxy, arylalkyl, heterocyclyl, and heteroaryl; wherein said cycloalkyl, aryl, alkyl, haloalkyl, haloalkyloxy, arylalkyl, heterocyclyl, or heteroaryl can be unsubstituted or substituted with one or more Z⁴;
or R³ and R⁴ together with the nitrogen atom to which they are attached form a saturated or unsaturated 5-, 6-, 7-, 8-, 9-, 10- or 11-membered nitrogen-containing monocycle or bicycle;
R⁵ is selected from the group consisting of hydrogen, alkyl, cycloalkyl, halo, aryl, arylalkyl, heterocyclyl, heteroaryl, hydroxyl, —OR⁸, cyano, amino, nitro, haloalkyl, haloalkyloxy, —C(O)R⁸, —NR⁸R⁹, —CO₂R⁹, —C(O)NR⁹R¹⁰, —S(O)₂R⁹, —S(O)₂NR⁹R¹⁰, —NR⁹C(O)R¹⁰, and —NR⁹S(O)₂R¹⁰;
or wherein two R⁵ together with the atom to which they are attached can form a saturated or unsaturated 5-, 6-, 7-, 8-, 9- or 10-membered ring;
wherein said ring, alkyl, cycloalkyl, aryl, arylalkyl, heterocyclyl, or heteroaryl can be unsubstituted or substituted with one or more Z⁵;
R⁶ is selected from the group consisting of hydrogen, alkyl, cycloalkyl, aryl, arylalkyl, heterocyclyl, heteroaryl, and —S(O)₂R⁹; wherein said alkyl, cycloalkyl, aryl, arylalkyl, heterocyclyl, or heteroaryl can be unsubstituted or substituted with one or more Z⁶;
R⁷ is selected from the group consisting of alkyl, cycloalkyl, aryl, arylalkyl, heterocyclyl, and heteroaryl; wherein said alkyl, cycloalkyl, aryl, arylalkyl, heterocyclyl, or heteroaryl can be unsubstituted or substituted with one or more Z⁷;
or R³ and R⁷ together with the atom to which they are attached form an unsaturated 5-, 6-, 7-, 8-, 9-, 10- or 11-membered nitrogen-containing monocycle or bicycle;
each R⁸ is independently selected from the group consisting of, alkyl, aryl, cycloalkyl, arylalkyl, heterocyclyl, heteroaryl;
each R⁹ is independently selected from the group consisting of hydrogen, alkyl, aryl, cycloalkyl, arylalkyl, heterocyclyl, heteroaryl;
each R¹⁰ is independently selected from the group consisting of hydrogen, alkyl, aryl, cycloalkyl, arylalkyl, heterocyclyl, heteroaryl;
each Z¹ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z² is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z³ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z⁴ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z⁵ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z⁶ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z⁷ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
or a solvate, hydrate, pharmaceutically acceptable salt, or prodrug thereof for use as a medicament.

12. A pharmaceutical composition comprising a compound of formula (I) or a stereoisomer, or tautomer thereof, 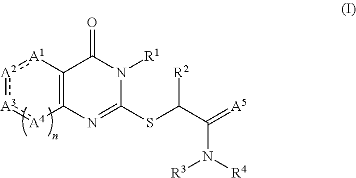
wherein,
each dotted line represents an optional double bond whereby maximally 2 non-adjacent dotted lines can form a double bond;
n is an integer selected from 0, 1, or 2;
A¹ is selected from the group consisting of CR⁵, S, N, NR⁶, and O;
A² is selected from the group consisting of CR⁵, S, N, NR⁶, and O;
A³ is selected from the group consisting of CR⁵, S, N, NR⁶, and O;
A⁴ is selected from the group consisting of CR⁵, S, N, NR⁶, and O;
wherein at most two of A¹ to A⁴ is selected from S, N, NR⁶, and O;
A⁵ is O or NR¹;
R¹ is selected from the group consisting of alkyl, cycloalkyl, alkenyl, aryl, arylalkyl, heterocyclyl, and heteroaryl; wherein said alkyl, cycloalkyl, alkenyl, aryl, arylalkyl, heterocyclyl, or heteroaryl can be unsubstituted or substituted with one or more Z¹;
R² is selected from the group consisting of hydrogen, alkyl, cycloalkyl, aryl, arylalkyl, heterocyclyl, and heteroaryl; wherein said alkyl, cycloalkyl, aryl, arylalkyl, heterocyclyl, or heteroaryl can be unsubstituted or substituted with one or more Z²;
R³ is selected from the group consisting of hydrogen, cycloalkyl, aryl, alkyl, haloalkyl, haloalkyloxy, arylalkyl, heterocyclyl, heteroaryl, —C(O)R⁸, —CO₂R⁹, —C(O)NR⁹R¹⁰, —S(O)₂R⁹, and —SO₂NR⁹R¹⁰; wherein said cycloalkyl, aryl, alkyl, arylalkyl, heterocyclyl, or heteroaryl can be unsubstituted or substituted with one or more Z³;
or R² and R³ together with the atom to which they are attached form a saturated or unsaturated 5-, 6-, 7-, 8- or 9-membered ring;
R⁴ is selected from the group consisting of hydrogen, cycloalkyl, aryl, alkyl, haloalkyl, haloalkyloxy, arylalkyl, heterocyclyl, and heteroaryl; wherein said cycloalkyl, aryl, alkyl, haloalkyl, haloalkyloxy, arylalkyl, heterocyclyl, or heteroaryl can be unsubstituted or substituted with one or more Z⁴;
or R³ and R⁴ together with the nitrogen atom to which they are attached form a saturated or unsaturated 5-, 6-, 7-, 8-, 9-, 10- or 11-membered nitrogen-containing monocycle or bicycle;
R⁵ is selected from the group consisting of hydrogen, alkyl, cycloalkyl, halo, aryl, arylalkyl, heterocyclyl, heteroaryl, hydroxyl, —OR^{S}, cyano, amino; nitro, haloalkyl, haloalkyloxy, —C(O)R⁸, —NR⁸R⁹, —CO₂R⁹, —C(O)NR⁹R¹⁰, —S(O)₂R⁹, —S(O)₂NR⁹R¹⁰, —NR⁹C(O)R¹⁰, and —NR⁹S(O)₂R¹⁰;
or wherein two R⁵ together with the atom to which they are attached can form a saturated or unsaturated 5-, 6-, 7-, 8-, 9- or 10-membered ring;
wherein said ring, alkyl, cycloalkyl, aryl, arylalkyl, heterocyclyl, or heteroaryl can be unsubstituted or substituted with one or more Z⁵;
R⁶ is selected from the group consisting of hydrogen, alkyl, cycloalkyl, aryl, arylalkyl, heterocyclyl, heteroaryl, and —S(O)₂R⁹; wherein said alkyl, cycloalkyl, aryl, arylalkyl, heterocyclyl, or heteroaryl can be unsubstituted or substituted with one or more Z⁶;
R⁷ is selected from the group consisting of alkyl, cycloalkyl, aryl, arylalkyl, heterocyclyl, and heteroaryl; wherein said alkyl, cycloalkyl, aryl, arylalkyl, heterocyclyl, or heteroaryl can be unsubstituted or substituted with one or more Z⁷;
or R³ and R⁷ together with the atom to which they are attached form an unsaturated 5-, 6-, 7-, 8-, 9-, 10- or 11-membered nitrogen-containing monocycle or bicycle;
each R⁸ is independently selected from the group consisting of, alkyl, aryl, cycloalkyl, arylalkyl, heterocyclyl, heteroaryl;
each R⁹ is independently selected from the group consisting of hydrogen, alkyl, aryl, cycloalkyl, arylalkyl, heterocyclyl, heteroaryl;
each R¹⁰ is independently selected from the group consisting of hydrogen, alkyl, aryl, cycloalkyl, arylalkyl, heterocyclyl, heteroaryl;
each Z¹ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z² is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z³ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z⁴ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z⁵ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z⁶ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
each Z⁷ is independently selected from the group consisting of halo, alkyl, haloalkyl, haloalkyloxy, cycloalkyl, cycloalkenyl, aryl, alkylaryl, heterocyclyl, heteroaryl, hydroxyl, —OR⁸, cyano, amino, —NR⁸R⁹, —C(O)₂R⁹, —C(O)NR⁹R¹⁰, —C(O)R⁸, —S(O)R⁹, —S(O)₂R⁹, —S(O)₂NR⁹R¹⁰, nitro;
and a pharmaceutically acceptable carrier.
